(19) United States
(12) Patent Application Publication
(10) Pub. No.: US 2022/0211048 A1
(43) Pub. Date: Jul. 7, 2022

(54) GENE TARGETS FOR NITROGEN FIXATION TARGETING FOR IMPROVING PLANT TRAITS

(71) Applicant: Pivot Bio, Inc., Berkeley, CA (US)

(72) Inventors: Karsten Temme, Berkeley, CA (US); Alvin Tamsir, Berkeley, CA (US); Sarah Bloch, Berkeley, CA (US); Neal Shah, Berkeley, CA (US); Jenny Johnson, Berkeley, CA (US); Bilge Ozaydin Eskiyenenturk, Berkeley, CA (US)

(21) Appl. No.: 17/605,374

(22) PCT Filed: Apr. 24, 2020
(86) PCT No.: PCT/US2020/029894

§ 371 Date: Oct. 21, 2021

Related U.S. Application Data
(60) Provisional application No. 62/838,158, filed on Apr. 24, 2019.

(51) Int. Cl. A01N 63/27; C05F 11/08; C12N 15/52

(52) U.S. Cl. CPC A01N 63/27; C12N 15/52; C05F 11/08

(57) ABSTRACT

A genetically engineered bacterium with a modification in one or more genes selected from: NAC, ptsH, iaaA, gltA, pga, sdiA, fimA1, fimA2, fimA3, fimA4, wzxE, bolA, iscR, fhuF, sodA, sodB, sodC, FNR, arcA, arcB, rpoS, sbnA, treA, treB, phoP, phoQ, yjjPB, ychM, dauA, actP, yusV1, yieL1, yieL2, yieL3, yieL4, pgaB, rafA, melA, uidA, manA, abfA, abnA, lacZ, and yusV2 is disclosed. Methods of use of the genetically engineered bacterium to provide fixed nitrogen to plants, and compositions including the bacterium are also provided.

### CROSS REFERENCE TO RELATED APPLICATIONS

This application claims priority to U.S. Provisional Application No. 62/838,158 filed on Apr. 24, 2019, which is herein incorporated by reference in its entirety.

### TECHNICAL FIELD

The present disclosure is related to genetically-engineered bacterial strains, and compositions thereof. Such bacterial strains, and compositions thereof, are useful for providing fixed nitrogen to plants.

### BACKGROUND OF THE INVENTION

Plants are linked to the microbiome via a shared metabolome. A multidimensional relationship between a particular crop trait and the underlying metabolome is characterized by a landscape with numerous local maxima. Optimizing from an inferior local maximum to another representing a better trait by altering the influence of the microbiome on the metabolome may be desirable for a variety of reasons, such as for crop optimization. Economically-, environmentally-, and socially-sustainable approaches to agriculture and food production are required to meet the needs of a growing global population. By 2050 the United Nations' Food and Agriculture Organization projects that total food production must increase by 70% to meet the needs of the growing population, a challenge that is exacerbated by numerous factors, including diminishing freshwater resources, increasing competition for arable land, rising energy prices, increasing input costs, and the likely need for crops to adapt to the pressures of a drier, hotter, and more extreme global climate.

One area of interest is in the improvement of nitrogen fixation. Nitrogen gas (N₂) is a major component of the atmosphere of Earth. In addition, elemental nitrogen (N) is an important component of many chemical compounds which make up living organisms. However, many organisms cannot use N₂ directly to synthesize the chemicals used in physiological processes, such as growth and reproduction. In order to utilize the N₂, the N₂ must be combined with hydrogen. The combining of hydrogen with N₂ is referred to as nitrogen fixation. Nitrogen fixation, whether accomplished chemically or biologically, requires an investment of large amounts of energy. In biological systems, an enzyme known as nitrogenase catalyzes the reaction which results in nitrogen fixation. An important goal of nitrogen fixation research is the extension of this phenotype to non-leguminous plants, particularly to important agronomic grasses such as wheat, rice, and maize. Despite enormous progress in understanding the development of the nitrogen-fixing symbiosis between rhizobia and legumes, the path to use that knowledge to induce nitrogen-fixing nodules on non-leguminous crops is still not clear. Meanwhile, the challenge of providing sufficient supplemental sources of nitrogen, such as in fertilizer, will continue to increase with the growing need for increased food production.

### SUMMARY OF THE INVENTION

The present disclosure provides a genetically engineered bacterium with a modification in a gene selected from: NAC, ptsH, iaaA, gltA, pga, sdiA, fimA1, fimA2, fimA3, fimA4, wzxE, bolA, iscR, fhuF, sodA, sodB, sodC, FNR, arcA, arcB, rpoS, sbnA, treA, treB, phoP, phoQ, yjjPB, ychM, dauA, actP, yusV1, yieL1, yieL2, yieL3, yieL4, pgab, rafA, melA, uidA, manA, abfA, abnA, lacZ, and yusV2. In some embodiments, the genetically engineered bacterium has a modification in a gene selected from: NAC, gltA, pga, ptsH, fimA1, fimA2, fimA3, fimA4, iscR, tonB, yusV1, yusV2, yusV3, yusV4, sbnA, fhuF, sodA, sodB, and sodC. In some embodiments, the genetically engineered bacterium has a modification in a gene selected from: NAC, gltA, pga, ptsH, fimA1, fimA2, fimA3, fimA4, sodA, sodB, and sodC. In some embodiments, the genetically engineered bacterium has a modification in a gene selected from: iscR, tonB, yusV1, yusV2, yusV3, yusV4, sbnA, and fhuF.

In some embodiments, the genetically engineered bacterium is a genetically engineered diazotrophic bacterium. In some embodiments, the genetically engineered bacterium is intergeneric.

In some embodiments, the genetically engineered microorganism is non-intergeneric. In some embodiments, the genetically engineered bacterium is able to fix atmospheric nitrogen in the presence of exogenous nitrogen. In some embodiments, the genetically engineered bacterium includes a modification in a nitrogen fixation genetic network. In some embodiments, the modification in a nitrogen fixation genetic network includes a modification in NifA, NifL, NifH, or any combination thereof. In some embodiments, the modification in NifA results in increased expression of NifA. In some embodiments, the modification in NifL results in decreased expression of NifL. In some embodiments, the modification in NifH results in increased expression of NifH. In some embodiments, the modification in a nitrogen fixation genetic network results in increased expression of Nif cluster genes. In some embodiments, the genetically engineered bacterium includes a modification in a nitrogen assimilation genetic network. In some embodiments, the modification in a nitrogen assimilation genetic network includes a modification in GlnE. In some embodiments, the modification in GlnE results in decreased activity of GlnE. In some embodiments, the modification in a nitrogen assimilation genetic network results in decreased activity of amtB.

The present disclosure provides a method of increasing the amount of atmospheric derived nitrogen in a plant, where the method includes a step of contacting the plant with a plurality of genetically engineered bacterium described herein. In some embodiments, the contacting step involves applying the plurality of genetically engineered bacterium to a seed of the plant. In some embodiments, the contacting step involves applying the plurality of genetically engineered bacterium to a seedling of the plant. In some embodiments, the contacting step involves applying the plurality of genetically engineered bacterium to the plant in a liquid formulation. In some embodiments, the contacting step involves applying the plurality of genetically engineered bacterium to the plant after planting but before harvest of the plant. In some embodiments, the contacting step involves applying the plurality of genetically engineered bacterium to the plant as a side dressing. In some embodiments, the contacting step involves applying the plurality of genetically engineered bacterium to the plant between one month and eight months after germination. In some embodiments, the contacting step involves applying the plurality of genetically engineered bacterium to the plant between two months and eight months after germination. In some embodiments, the contacting step involves applying the plurality of genetically engineered bacterium to the plant between one month and three months after germination. In some embodiments, the contacting step involves applying the plurality of genetically engineered bacterium to the plant between three months and six months after germination. In some embodiments, the plant is a cereal plant. In some embodiments, the plant is a corn plant. In some embodiments, the plant is a rice plant. In some embodiments, the plant is a wheat plant. In some embodiments, the plant is a soy plant.

The present disclosure provides a composition which includes a seed, and a seed coating; where the seed coating includes a plurality of the genetically engineered bacteria as described herein. In some embodiments, the seed is a cereal seed. In some embodiments, the seed is selected from the group consisting of: a corn seed, a wheat seed, a rice seed, a soy seed, a rye seed and a Sorghum seed.

The present disclosure provides a composition which includes a plant and a plurality of the genetically engineered bacterium described herein. In some embodiments, the plant is a seedling. In some embodiments, the plant is a cereal plant. In some embodiments, the plant is selected from corn, rice, wheat, soy, rye, and Sorghum.

### INCORPORATION BY REFERENCE

All publications, patents, and patent applications mentioned in this specification are herein incorporated by reference to the same extent as if each individual publication, patent, or patent application was specifically and individually indicated to be incorporated by reference. In addition, the disclosure of International Publication No. WO 2019/084342 is incorporated by reference herein in its entirety.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-B depict enrichment and isolation of nitrogen fixing bacteria. (A) Nfb agar plate was used to isolate single colonies of nitrogen fixing bacteria. (B) Semi-solid Nfb agar casted in Balch tube. The arrow points to pellicle of enriched nitrogen fixing bacteria.

FIG. 2 depicts a representative nifH PCR screen. Positive bands were observed at ˜350 bp for two colonies in this screen. Lower bands represent primer-dimers.

FIG. 3 depicts an example of a PCR screen of colonies from CRISPR-Cas-selected mutagenesis. CI006 colonies were screened with primers specific for the nifL locus. The wild type PCR product is expected at ˜2.2 kb, whereas the mutant is expected at ˜1.1 kb. Seven of ten colonies screened unambiguously show the desired deletion.

FIGS. 4A-B depict in vitro phenotypes of various strains. The Acetylene Reduction Assay (ARA) activities of mutants of strain CI137 grown in nitrogen fixation media supplemented with 0 mM (FIG. 4A) or 5 mM (FIG. 4B) ammonium phosphate. All strains are compared to 137-2084 (Parent strain).

FIGS. 5A-B provide the total ammonium excretion (FIG. 5B) and ammonium excretion profile across time (FIG. 5A) for the strains from FIGS. 4A-B. All strains are compared to 137-2084 (Parent strain).

FIGS. 6A-B depict in vitro phenotypes of various strains. ARA activities of mutants of strain CI137 grown in nitrogen fixation media supplemented with 0 mM (FIG. 6A) or 5 mM (FIG. 6B) ammonium phosphate. All strains are compared to 137-2084 (Parent strain).

FIGS. 7A-B provide the total ammonium excretion (FIG. 7B) and ammonium excretion profile across time (FIG. 7A) for the strains from FIGS. 6A-B. All strains are compared to 137-2084 (Parent strain).

FIGS. 8A-B depict in vitro phenotypes of various strains. ARA activities of siderophore gene mutants of strain CI137 grown in nitrogen fixation media supplemented with 0 mM (FIG. 8A) or 5 mM (FIG. 8B) ammonium phosphate. All strains are compared to 137 (Parent strain).

FIGS. 9A-B provide the total ammonium excretion (FIG. 9B) and ammonium excretion profile across time (FIG. 9A) for the strains from FIGS. 8A-B. All strains are compared to 137 (Parent strain).

FIGS. 10A-B depict in vitro phenotypes of various strains. ARA activities of siderophore gene mutants of strain CI137 grown in nitrogen fixation media supplemented with 0 mM (FIG. 10A) or 5 mM (FIG. 10B) ammonium phosphate. All strains are compared to 137-2084 (Parent strain).

FIGS. 11A-B provide the total ammonium excretion (FIG. 11B) and ammonium excretion profile across time (FIG. 11A) for the strains from FIGS. 10A-B. All strains are compared to 137-2084 (Parent strain).

FIGS. 12A-B depict in vitro phenotypes of various strains. ARA activities of oxygen tolerance gene mutants of strain CI137 grown in nitrogen fixation media supplemented with 0 mM (FIG. 12A) or 5 mM (FIG. 12B) ammonium phosphate. All strains are compared to 137 (Parent strain).

FIGS. 13A-B provide the total ammonium excretion (FIG. 13B) and ammonium excretion profile across time (FIG. 13A) for the strains from FIGS. 12A-B. All strains are compared to 137 (Parent strain).

FIGS. 14A-B depict in vitro phenotypes of various strains. ARA activities of oxygen tolerance gene mutants of strain CI137 grown in nitrogen fixation media supplemented with 0 mM (FIG. 14A) or 5 mM (FIG. 14B) ammonium phosphate. All strains are compared to 137-2084 (Parent strain).

FIGS. 15A-B provide the total ammonium excretion (FIG. 15B) and ammonium excretion profile across time (FIG. 15A) for the strains from FIGS. 14A-B. All strains are compared to 137-2084 (Parent strain).

FIGS. 16A-B depict in vitro phenotypes of various strains. ARA activities of siderophore gene mutants of strain CI1021 grown in nitrogen fixation media supplemented with 0 mM (FIG. 16A) or 5 mM (FIG. 16B) ammonium phosphate. All strains are compared to 1021 (Parent strain).

FIGS. 17A-B provide the total ammonium excretion (FIG. 17B) and ammonium excretion profile across time (FIG. 17A) for the strains from FIGS. 16A-B. All strains are compared to 1021 (Parent strain).

FIGS. 18A-B depict in vitro phenotypes of various strains. ARA activities of siderophore gene mutants of strain CI1021 grown in nitrogen fixation media supplemented with 0 mM (FIG. 18A) or 5 mM (FIG. 18B) ammonium phosphate. All strains are compared to 1021-1615 (Parent strain).

FIGS. 19A-B provide the total ammonium excretion (FIG. 19B) and ammonium excretion profile across time (FIG. 19A) for the strains from FIGS. 18A-B. All strains are compared to 1021-1615 (Parent strain).

FIGS. 20A-B depict in vitro phenotypes of various strains. ARA activities of oxygen tolerance gene mutants of strain CI1021 grown in nitrogen fixation media supplemented with 0 mM (FIG. 20A) or 5 mM (FIG. 20B) ammonium phosphate. All strains are compared to 1021 (Parent strain). Assay carried out under 1% oxygen.

FIGS. 21A-B provide the total ammonium excretion (FIG. 21B) and ammonium excretion profile across time (FIG. 21A) for the strains from FIGS. 20A-B. All strains are compared to 1021 (Parent strain). Assay carried out under 1% oxygen.

FIGS. 22A-B depict in vitro phenotypes of various strains. ARA activities of oxygen tolerance gene mutants of strain CI1021 grown in nitrogen fixation media supplemented with 0 mM (FIG. 22A) or 5 mM (FIG. 22B) ammonium phosphate. All strains are compared to 1021-1615 (Parent strain). Assay carried out under 1% oxygen.

FIGS. 23A-B provide the total ammonium excretion (FIG. 23B) and ammonium excretion profile across time (FIG. 23A) for the strains from FIGS. 22A-B. All strains are compared to 1021-1615 (Parent strain). Assay carried out under 1% oxygen.

FIGS. 24A-B depict in vitro phenotypes of various strains. ARA activities of NAC gene mutants of strain CI137 grown in nitrogen fixation media supplemented with 0 mM (FIG. 24A) or 5 mM (FIG. 24B) ammonium phosphate.

FIGS. 25A-B provide the total ammonium excretion (FIG. 25B) and ammonium excretion profile across time (FIG. 25A) for the strains from FIGS. 24A-B.

FIGS. 26A-B depict in vitro phenotypes of various strains. ARA activities of NAC gene mutants of strain CI1021 grown in nitrogen fixation media supplemented with 0 mM (FIG. 26A) or 5 mM (FIG. 26B) ammonium phosphate. GlnA operon upregulation increased nitrogen fixation by ˜2-3 fold in the ΔnifL::Prm2 strains. GlnA operon upregulation increased fixation by ˜200-fold in wt 1021 strain.

FIGS. 27A-B provide the total ammonium excretion (FIG. 27B) and ammonium excretion profile across time (FIG. 27A) for the strains from FIGS. 26A-B. GlnA operon upregulation increased nitrogen excretion by ˜90-fold in wt and 3-fold in repressed background.

### DETAILED DESCRIPTION OF THE INVENTION

The terms “polynucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner according to base complementarity. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the enzymatic cleavage of a polynucleotide by an endonuclease. A second sequence that is complementary to a first sequence is referred to as the “complement” of the first sequence. The term “hybridizable” as applied to a polynucleotide refers to the ability of the polynucleotide to form a complex that is stabilized via hydrogen bonding between the bases of the nucleotide residues in a hybridization reaction.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. Sequence identity, such as for the purpose of assessing percent complementarity, may be measured by any suitable alignment algorithm, including but not limited to the Needleman-Wunsch algorithm (see e.g. the EMBOSS Needle aligner available at ebi.ac.uk/Tools/psa/emboss needle/nucleotide.html on the World Wide Web, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at.ebi.ac.uk/Tools/psa/emboss_water/nucleotide.html on the World Wide Web, optionally with default settings). Optimal alignment may be assessed using any suitable parameters of a chosen algorithm, including default parameters.

In general, “stringent conditions” for hybridization refer to conditions under which a nucleic acid having complementarity to a target sequence predominantly hybridizes with a target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, may be calculated as the number of exact matches between two aligned sequences divided by the length of the shorter sequences and multiplied by 100. In some cases, the percent identity of a test sequence and a reference sequence, whether nucleic acid or amino acid sequences, may be calculated as the number of exact matches between two aligned sequences divided by the length of the reference sequence and multiplied by 100. Percent identity may also be determined, for example, by comparing sequence information using the advanced BLAST computer program, including version 2.2.9, available from the National institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:40:3-410 (1990): Karlin. And Altschul, Proc. Natl. Acad. Sci. USA. 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, at least 98% or at least 99%.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified. For example, an amino acid polymer that has disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “about” is used synonymously with the term “approximately.” Illustratively, the use of the term “about” with regard to an amount indicates that values slightly outside the cited values, e.g., plus or minus 0.1% to 10%.

The term “biologically pure culture” or “substantially pure culture” refers to a culture of a bacterial species described herein containing no other bacterial species in quantities sufficient to interfere with the replication of the culture or be detected by normal bacteriological techniques.

“Plant productivity” refers generally to any aspect of growth or development of a plant that is a reason for which the plant is grown. For food crops, such as grains or vegetables, “plant productivity” can refer to the yield of grain or fruit harvested from a particular crop. As used herein, improved plant productivity refers broadly to improvements in yield of grain, fruit, flowers, or other plant parts harvested for various purposes, improvements in growth of plant parts, including stems, leaves and roots, promotion of plant growth, maintenance of high chlorophyll content in leaves, increasing fruit or seed numbers, increasing fruit or seed unit weight, reducing NO₂ emission due to reduced nitrogen fertilizer usage and similar improvements of the growth and development of plants.

Microbes in and around food crops can influence the traits of those crops. Plant traits that may be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

As used herein, a “control sequence” refers to an operator, promoter, silencer, or terminator.

In some embodiments, native or endogenous control sequences of genes of the present disclosure are replaced with one or more intrageneric control sequences.

As used herein, “introduced” refers to the introduction by means of modern biotechnology, and not a naturally occurring introduction.

In some embodiments, the bacteria of the present disclosure have been modified such that they are not naturally occurring bacteria.

In some embodiments, the bacteria of the present disclosure are present in the plant in an amount of at least 10³ colony-forming units (cfu), 10⁴ cfu, 10⁵ cfu, 10⁶ cfu, 10⁷ cfu, 10⁸ cfu, 10⁹ cfu, 10¹⁰ cfu, 10¹¹ cfu, or 10¹² cfu per gram of fresh or dry weight of the plant. In some embodiments, the bacteria of the present disclosure are present in the plant in an amount of at least about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, or about 10¹² cfu per gram of fresh or dry weight of the plant. In some embodiments, the bacteria of the present disclosure are present in the plant in an amount of at least 10³ to 10⁹, 10³ to 10⁷, 10³ to 10⁵, 10⁵ to 10⁹, 10⁵ to 10⁷, 10⁶ to 10¹⁰, 10⁶ to 10⁷ cfu per gram of fresh or dry weight of the plant. In some embodiments, the bacteria of the present disclosure are present in the plant in an amount of at least about 10³ to about 10⁹, about 10³ to about 10⁷, about 10³ to about 10⁵, about 10⁵ to about 10⁹, about 10⁵ to about 10⁷, about 10⁶ to about 10¹⁰, about 10⁶ to about 10⁷ cfu per gram of fresh or dry weight of the plant.

In some embodiments, the bacteria of the present disclosure are present in the plant in an amount of about 10³ cfu to about 10²⁰ cfu. For example, about 10³ cfu to about 10¹⁵ cfu, about 10³ cfu to about 10¹⁰ cfu, about 10³ cfu to about 10⁵ cfu, about 10¹⁵ cfu to about 10²⁰ cfu, about 10¹⁰ cfu to about 10²⁰ cfu, or about 10⁵ cfu to about 10²⁰ cfu. Fertilizers and exogenous nitrogen of the present disclosure can comprise the following nitrogen-containing molecules: ammonium, nitrate, nitrite, ammonia, glutamine, etc. Nitrogen sources of the present disclosure can include anhydrous ammonia, ammonia sulfate, urea, diammonium phosphate, urea-form, monoammonium phosphate, ammonium nitrate, nitrogen solutions, calcium nitrate, potassium nitrate, sodium nitrate, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field, and/or media at concentrations greater than about 4 mM nitrogen, as disclosed by Kant et al. (2010. J. Exp. Biol. 62(4):1499-1509), which is incorporated herein by reference.

As used herein, “introduced genetic material” means genetic material that is added to, and remains as a component of, the genome of the recipient.

In some embodiments, the nitrogen fixation and assimilation genetic regulatory network comprises polynucleotides encoding genes and non-coding sequences that direct, modulate, and/or regulate microbial nitrogen fixation and/or assimilation and can comprise polynucleotide sequences of the nif cluster (e.g., nifA, nifB, nifC, . . . nifZ), polynucleotides encoding nitrogen regulatory protein C, polynucleotides encoding nitrogen regulatory protein B, polynucleotide sequences of the gln cluster (e.g. glnA and glnD), draT, and ammonia transporters/permeases. In some embodiments, the Nif cluster can comprise NifB, NifH, NifD, NifK, NifE, NifN, NifX, hesA, and NifV. In some embodiments, the Nif cluster can comprise a subset of NifB, NifH, NifD, NifK, NifE, NifN, NifX, hesA, and NifV.

In some embodiments, fertilizer of the present disclosure comprises at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% nitrogen by weight.

In some embodiments, fertilizer of the present disclosure comprises at least about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% nitrogen by weight.

In some embodiments, fertilizer of the present disclosure comprises about 5% to about 50%, about 5% to about 75%, about 10% to about 50%, about 10% to about 75%, about 15% to about 50%, about 15% to about 75%, about 20% to about 50%, about 20% to about 75%, about 25% to about 50%, about 25% to about 75%, about 30% to about 50%, about 30% to about 75%, about 35% to about 50%, about 35% to about 75%, about 40% to about 50%, about 40% to about 75%, about 45% to about 50%, about 45% to about 75%, or about 50% to about 75% nitrogen by weight.

In some embodiments, the increase of nitrogen fixation and/or an increase in the production of 1% or more of the nitrogen in the plant is measured relative to control plants, which have not been exposed to the bacteria of the present disclosure. All increases or decreases in an activity of the bacteria (e.g., any of the activities of the bacteria as described herein including the nitrogen-fixing activity of the bacteria, the nitrogen assimilation activity of the bacteria, the plant-colonizing activity of the bacteria, the ammonium excretion activity of the bacteria, and the iron uptake activity of the bacteria) are measured relative to control bacteria. All increases or decreases in the productivity or a property of the plants (e.g., increases or decreases in plant growth, yield, NO₂ emission, and nitrogen uptake) are measured relative to control plants.

As used herein, a “constitutive promoter” is a promoter, which is active under most conditions and/or during most development stages. There are several advantages to using constitutive promoters in expression vectors used in biotechnology, such as: high level of production of proteins used to select transgenic cells or organisms; high level of expression of reporter proteins or scorable markers, allowing easy detection and quantification; high level of production of a transcription factor that is part of a regulatory transcription system; production of compounds that requires ubiquitous activity in the organism; and production of compounds that are required during all stages of development. Non-limiting exemplary constitutive promoters include, CaMV 35S promoter, opine promoters, ubiquitin promoter, alcohol dehydrogenase promoter, etc.

As used herein, a “non-constitutive promoter” is a promoter which is active under certain conditions, in certain types of cells, and/or during certain development stages. For example, tissue specific, tissue preferred, cell type specific, cell type preferred, inducible promoters, and promoters under development control are non-constitutive promoters. Examples of promoters under developmental control include promoters that preferentially initiate transcription in certain tissues.

As used herein, “inducible” or “repressible” promoter is a promoter which is under chemical or environmental factors control. Examples of environmental conditions that may affect transcription by inducible promoters include anaerobic conditions, certain chemicals, the presence of light, acidic or basic conditions, etc.

As used herein, a “tissue specific promoter” is a promoter that initiates transcription only in certain tissues. Unlike constitutive expression of genes, tissue-specific expression is the result of several interacting levels of gene regulation. As such, in the art sometimes it is preferable to use promoters from homologous or closely related species to achieve efficient and reliable expression of transgenes in particular tissues. This is one of the main reasons for the large amount of tissue-specific promoters isolated from particular tissues found in both scientific and patent literature.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the disclosure can be operably linked, either directly or indirectly, 5′ to the target mRNA, or 3′ to the target mRNA, or within the target mRNA, or a first complementary region is 5′ and its complement is 3′ to the target mRNA.

In general, the term “genetic modification” or “modification in a gene” refers to any change introduced into a polynucleotide sequence relative to a reference polynucleotide, such as a reference genome or portion thereof, or reference gene or portion thereof. A genetic modification may be referred to as a “mutation,” and a sequence or organism comprising a genetic modification may be referred to as a “genetic variant” or “mutant”. Genetic modifications can have any number of effects, such as the increase or decrease of some biological activity, including gene expression, metabolism, and cell signaling. Genetic modifications can be specifically introduced to a target site, or introduced randomly.

Provided herein is a genetically engineered bacterium with a modification in a gene selected from: NAC, ptsH, iaaA, gltA, pga, sdiA, fimA1, fimA2, fimA3, fimA4, wzxE, bolA, iscR, fhuF, sodA, sodB, sodC, FNR, arcA, arcB, rpoS, sbnA, treA, treB, phoP, phoQ, yjjPB, ychM, dauA, actP, yusV1, yieL1, yieL2, yieL3, yieL4, pgab, rafA, melA, uidA, manA, abfA, abnA, lacZ, and yusV2. In some embodiments, the genetically engineered bacterium has a modification in a gene selected from: NAC, gltA, pga, ptsH, fimA1, fimA2, fimA3, fimA4, iscR, tonB, yusV1, yusV2, yusV3, yusV4, sbnA, fhuF, sodA, sodB, and sodC. In some embodiments, the genetically engineered bacterium has a modification in a gene selected from: NAC, gltA, pga, ptsH, fimA1, fimA2, fimA3, fimA4, sodA, sodB, and sodC. In some embodiments, the genetically engineered bacterium has a modification in a gene selected from: iscR, tonB, yusV1, yusV2, yusV3, yusV4, sbnA, and fhuF. Bacteria described herein can be used in a method of improving plant nitrogen uptake. Such a method can include the step of contacting a plant with a plurality of the bacterium. Also provided are compositions containing a plurality of the bacterium in the form of a seed coating for a seed or in combination with a plant. Bacteria with modifications in the identified genes can exhibit one or more of the following: increased nitrogen fixation, increased ammonium excretion, increased colonization, increased iron transport, increased oxygen tolerance, and/or increased desiccation tolerance relative to bacteria lacking modifications in the identified genes.

### Regulation of Nitrogen Fixation

One trait that can be improved through the use of the genetically engineered bacteria described herein is nitrogen fixation. While some endophytes have the genetics necessary for fixing nitrogen in pure culture, the fundamental technical challenge is that wild-type endophytes of cereals and grasses stop fixing nitrogen in fertilized fields. The application of chemical fertilizers and residual nitrogen levels in field soils signal the microbe to shut down the biochemical pathway for nitrogen fixation. Changes to the transcriptional and post-translational levels of nitrogen fixation regulatory network are required to develop a microbe capable of fixing and transferring nitrogen to corn in the presence of fertilizer. Thus, in some embodiments, the genetically engineered bacterium contains a modification of one or more genes of the nitrogen fixation regulatory network.

In order to utilize elemental nitrogen (N) for chemical synthesis, life forms combine nitrogen gas (N₂) available in the atmosphere with hydrogen in a process known as nitrogen fixation. Not all organisms are able to carry out nitrogen fixation. Rather, nitrogen fixation is limited to diazatrophs (bacteria and archaea that fix atmospheric nitrogen gas) that contain the genetic machinery for carrying out nitrogen fixation. Thus, in some embodiments, the genetically engineered bacterium is a diazotroph. Because of the energy-intensive nature of biological nitrogen fixation, diazotrophs have evolved sophisticated and tight regulation of the nif gene cluster, which codes for the machinery responsible for carrying out nitrogen fixation, in response to environmental oxygen and available nitrogen. To increase nitrogen fixation, it is desirable to increase expression of Nif cluster genes. Shamseldin (2013. Global J. Biotechnol. Biochem. 8(4):84-94) discloses detailed descriptions of nif genes and their products, and is incorporated herein by reference. Thus, in some embodiments, the modification in the genetically engineered bacterium's nitrogen fixation regulatory network results in increased expression of one or more Nif cluster genes.

In Proteobacteria, regulation of nitrogen fixation centers around the 654-dependent enhancer-binding protein NifA, the positive transcriptional regulator of the nif cluster whose activation would result in the increased expression of Nif cluster genes. Thus, in some embodiments, the modification in the genetically engineered bacterium's nitrogen fixation network includes a modification in NifA, which, in some embodiments, results in increased expression of NifA. Intracellular levels of active NifA are controlled by two key factors: transcription of the nifLA operon, and inhibition of NifA activity by protein-protein interaction with NifL whose activation would result in decreased expression of Nif cluster genes. Thus, in some embodiments, the modification in the genetically engineered bacterium's nitrogen fixation network includes a modification in NifL, which, in some embodiments, results in decreased expression of NifL.

In addition to regulating the transcription of the nif gene cluster, many diazotrophs have evolved a mechanism for the direct post-translational modification and inhibition of the nitrogenase enzyme itself, known as nitrogenase shutoff. This is mediated by ADP-ribosylation of the Fe protein (NifH) under nitrogen-excess conditions, which disrupts its interaction with the MoFe protein complex (NifDK) and abolishes nitrogenase activity. To avoid nitrogenase shutoff, it is desirable to increase the available NifH to interact with the MoFe protein complex. Thus, in some embodiments, the modification in the genetically engineered bacterium's nitrogen fixation network includes a modification in NifH, which, in some embodiments, results in increased expression of NifH.

Nitrogen fixation may also be responsive to intracellular, or extracellular, levels of ammonia, urea or nitrates. It is desirable to avoid nitrogen assimilation to avoid its feedback implications on the nitrogen fixation network. Thus, in some embodiments, the genetically engineered bacterium contains a modification in a nitrogen assimilation genetic network.

Ammonia uptake from the environment can be reduced by decreasing the expression level of amtB protein. Without intracellular ammonia, the endophyte is not able to sense the high level of ammonia, preventing the down-regulation of nitrogen fixation genes. Thus, in some embodiments, the modification in the nitrogen assimilation genetic network includes a modification in amtB, which, in some embodiments, results in decreased expression of amtB. Any ammonia that manages to get into the intracellular compartment is converted into glutamine. Intracellular glutamine level is the major currency of nitrogen sensing. Decreasing the intracellular glutamine level prevents the cells from sensing high ammonium levels in the environment. This effect can be achieved by increasing the expression level of glutaminase, an enzyme that converts glutamine into glutamate. In addition, intracellular glutamine can also be reduced by decreasing glutamine synthase (an enzyme that converts ammonia into glutamine). In diazotrophs, fixed ammonia is quickly assimilated into glutamine and glutamate to be used for cellular processes. Disruptions to ammonia assimilation may enable diversion of fixed nitrogen to be exported from the cell as ammonia. The fixed ammonia is predominantly assimilated into glutamine by glutamine synthetase (GS), encoded by glnA, and subsequently into glutamine by glutamine oxoglutarate aminotransferase (GOGAT). In some examples, glnS encodes a glutamine synthetase. GS is regulated post-translationally by GS adenylyl transferase (GlnE), a bi-functional enzyme encoded by glnE that catalyzes both the adenylylation and de-adenylylation of GS through activity of its adenylyl-transferase (AT) and adenylyl-removing (AR) domains, respectively. Under nitrogen limiting conditions, glnA is expressed, and GlnE's AR domain de-adynylylates GS, allowing it to be active. To disrupt ammonia assimilation and allow fixed nitrogen to be exported from the cell, it is desirable to decrease this activity of GlnE. Thus, in some embodiments, the genetically engineered bacterium's modification in a nitrogen assimilation genetic network includes a modification in GlnE, which, in some embodiments, results in decreased activity of GlnE.

The phosphoenolpyruvate-dependent sugar phosphotransferase system (PTS) is a major carbohydrate transport system in bacteria. The PTS catalyzes the phosphorylation of sugar substrates at the same time as they are translocated across the cell membrane. The phosphoryl group from phosphoenolpyruvate (PEP) is transferred to the phosphocarrier protein HPr by enzyme I, and then phospho-HPr then transfers it to the PTS EIIA domain. Phosphocarrier protein HPr is encoded by the ptsH gene. In some embodiments, the genetically engineered bacterium's modification is a modification in the ptsH gene, such as a substitution of the promoter regulating ptsH expression. In some embodiments, the genetically engineered bacterium's modification in the ptsH gene results in increased sugar transport. In some embodiments, the modification results in upregulation of phosphocarrier protein HPr.

L-asparaginase (LA), encoded by iaaA, catalyzes the degradation of asparagine amino acid into ammonia and aspartate. In some embodiments, the genetically engineered bacterium's modification is a modification in the iaaA gene. In some embodiments, the genetically engineered bacterium's modification in the iaaA gene results in decreased assimilation of ammonia and/or increased ammonia excretion. In some embodiments, the modification results in upregulation of L-asparaginase.

Citrate synthase (E.C. 2.3.3.1), encoded by the gltA gene, is involved in the first step of the citric acid cycle. It catalyzes the condensation reaction of the two-carbon acetate residue from acetyl coenzyme A and a molecule of four-carbon oxaloacetate to form the six-carbon citrate. In some embodiments, the genetically engineered bacterium's modification is a modification in the gltA gene such as a deletion of the gltA gene. In some embodiments, the genetically engineered bacterium's modification in the gltA gene results in increased ammonia excretion.

Methods for imparting new microbial phenotypes can be performed at the transcriptional, translational, and post-translational levels. The transcriptional level includes changes at the promoter (such as changing sigma factor affinity or binding sites for transcription factors, including deletion of all or a portion of the promoter) or changing transcription terminators and attenuators. The translational level includes changes at the ribosome binding sites and changing mRNA degradation signals. The post-translational level includes mutating an enzyme's active site and changing protein-protein interactions. These changes can be achieved in a multitude of ways. Reduction of expression level (or complete abolishment) can be achieved by swapping the native ribosome binding site (RBS) or promoter with another with lower strength/efficiency. ATG start sites can be swapped to a GTG, TTG, or CTG start codon, which results in reduction in translational activity of the coding region. Complete abolishment of expression can be done by knocking out (deleting) the coding region of a gene. Frameshifting the open reading frame (ORF) likely will result in a premature stop codon along the ORF, thereby creating a non-functional truncated product. Insertion of in-frame stop codons will also similarly create a non-functional truncated product. Addition of a degradation tag at the N or C terminal can also be done to reduce the effective concentration of a particular gene.

Conversely, an increased expression level of the genes described herein can be achieved by using a stronger promoter. To ensure high promoter activity during high nitrogen level condition (or any other condition), a transcription profile of the whole genome in a high nitrogen level condition could be obtained and active promoters with a desired transcription level can be chosen from that dataset to replace the weak promoter. Weak start codons can be swapped out with an ATG start codon for better translation initiation efficiency. Weak ribosomal binding sites (RBS) can also be swapped out with a different RBS with higher translation initiation efficiency. In addition, site specific mutagenesis can also be performed to alter the activity of an enzyme.

Increasing the level of nitrogen fixation that occurs in a plant can lead to a reduction in the amount of chemical fertilizer needed for crop production and reduce greenhouse gas emissions (e.g., nitrous oxide).

### Increasing the Activity of Nitrogen Fixation

Nitrogenases are enzymes responsible for catalyzing nitrogen fixation. There are three types of nitrogenase found in various nitrogen-fixing bacteria: molybdenum (Mo) nitrogenase, vanadium (V) nitrogenase, and iron-only (Fe) nitrogenase. Nitrogenases are two-component systems made up of Component I (also known as dinitrogenase) and Component II (also known as dinitrogenase reductase). Component I is a MoFe protein in molybdenum nitrogenase, a VFe protein in vanadium nitrogenase, and a Fe protein in iron-only nitrogenase. Component II is a Fe protein that contains an iron-sulfur (Fe—S) cluster.

Varying the supply of cofactors can increase nitrogen fixation. Cofactor supply may be affected by iron uptake. Iron uptake may be influenced by the tonB transport system. In some cases, influencing iron uptake may be achieved by upregulating tonB transport system genes. Some examples of tonB transport system genes include, but are not limited to, tonB and exbAB. In some cases, iron uptake may be influenced by siderophores which increase iron uptake in microbes and plants. In some cases, influencing iron uptake can be achieved by upregulating siderophore biosynthesis genes. Some examples of siderophore biosynthesis genes include, but are not limited to, yhfA, yusV, sbnA, fiu, yfiZ, and fur. Other genetic modifications which can regulate iron availability include iscR and fhuF.

Increased nitrogen fixation can also be achieved by increasing expression of nif cluster genes. Increased expression of nif cluster genes can be accomplished in multiple different ways. In some embodiments, the transcription of the nif cluster(s) can be increased by inserting strong promoters upstream of a nifHDK or nifDK operon. In some embodiments, increased expression of nif cluster genes is achieved by increasing the copy number of the genes in the genome. These additional copies of the genes can either be placed under the control of the native promoter or a strong constitutive promoter.

Another way to increase nitrogen fixation is to increase the number of nitrogenase enzymes per cell by increasing nif cluster transcription. Nif cluster transcription can be increased by increasing nifA transcription. In some cases, nif cluster transcription can be influenced by increasing the copy number of a nifA gene in the genome.

Nif cluster transcription can also be increased by increasing NifA translation. In some cases, NifA translation can be increased by increasing the strength of the ribosome binding site in the nifA gene.

Nif cluster transcription can also be increased by expressing the cluster under the control of a mutant form of NifA. Mutant forms of NifA can be obtained by subjecting the gene to mutagenesis and identifying a mutant that has increased translation efficiency or a mutant that expresses a corresponding protein with increased activity.

Increased nitrogen fixation can also be achieved by altering the cell's oxygen sensitivity. Oxygen sensitivity may be influenced by reducing oxygen sensing. In some cases, reducing oxygen sensing may be by disrupting oxygen-sensing genes. Some examples of oxygen-sensing genes include, but are not limited to, nifT/fixU, fixJ and fixL. Other genes that may be modified to alter oxygen sensitivity include sodA, sodB, sodC, FNR, arcA, and arcB.

In some cases, oxygen sensitivity can be influenced by keeping cytosolic oxygen levels low by promoting cytochrome bd-mediated respiration. In some cases, oxygen sensitivity can be influenced by upregulating genes encoding cytochrome bd oxidase and/or knocking out alternative cytochrome systems. Some examples of genes encoding cytochrome bd genes include, but are not limited to, cydABX, cydAB, and cydX. In some cases, nitrogenase can be protected from oxidation by altering redox balance in the cell. Redox balance can be altered through ROS scavenging. One strategy for accomplishing ROS scavenging is to upregulate relevant genes. Some examples of ROS scavenging genes include, but are not limited, to grxABCD, trxA, trxC, and tpx.

In some cases, oxygen sensitivity can be influenced by scavenging free oxygen. In some cases, scavenging free oxygen can be achieved by upregulating bacterial hemoglobin genes. An example of a hemoglobin gene includes, but is not limited to, glbN. In some cases, scavenging free oxygen can be achieved by upregulating fixNOPQ genes that code for a high-affinity heme-copper cbb3-type oxidase.

In some cases, modification of nifA can be beneficial in increasing nitrogenase expression. In some cases, it can be beneficial to modify nifA to increase nifA gene copy numbers in a cell. nifA gene copy numbers may be increased by insert multiple copies of a nifA gene in front of constitutively expressing promoters. In some cases, attaching a nifA gene copy to one or more housekeeping operons can increase an overall number of nifA genes in a cell. In some cases, strains that can be utilized in this process of increasing nitrogenase expression can include, but are not limited to, Rahnella aquatilis and Klebsiella variicola strains.

In some cases, modification of a nitrogenase operon can be beneficial in increasing nitrogenase expression. In some cases, it can be beneficial to upregulate nitrogenase operons to increase nitrogenase transcription. In some cases, promoters from within the bacterium that are active when the bacterium is colonizing the rhizosphere can be inserted in front of nitrogenase operons to upregulate nitrogenase operons. In some cases, nifL can be deleted within nitrogenase operons to upregulate nitrogenase operons. In some cases, nifA can be deleted within nitrogenase operons to upregulate nitrogenase operons. In some cases, nifA and nifL can be deleted within nitrogenase operons to upregulate nitrogenase operons. In some cases, multiple promoters can be placed directly in front of nifHDK genes to circumvent nifA transcription control. In some cases, strains that can be utilized in this process of increasing nitrogenase expression can include, but are not limited to, Rahnella aquatilis and Klebsiella variicola strains.

In some cases, modification of glnE can be beneficial in increasing ammonium excretion. In some cases, a conserved aspartate-amino acid-aspartate (DXD) motif on AR domain of glnE can be changed. An amino acid denoted as “X” indicates that the amino acid can be any amino acid including naturally occurring amino acids (e.g., a-amino acids), unnatural amino acids, modified amino acids, and non-natural amino acids. It can also include both D- and L-amino acids. In some cases, changing a conserved DXD residue on AR domain of glnE can be used to remove de-adenylylation activity from glnE. In some cases, a D residue may be replaced on a DXD motif in the AR region of glnE. In some cases, the replacement of a D residue on a DXD motif in the AR region of glnE can leave the GlnB binding site intact so as to allow for regulation of adenylation activity while decreasing or preventing AR activity. In some cases, strains that can be utilized in this process of increasing ammonium excretion can include, but are not limited to, Rahnella aquatilis, Kosakonia sacchari, and Klebsiella variicola strains.

### Increasing Nitrogen Fixation Through Assimilation

The amount of nitrogen provided to a microbe-associated plant can be increased by decreasing the nitrogen assimilation in the microbe. Thus, in some embodiments, the genetically engineered bacterium includes a modification in a nitrogen assimilation network which, in some embodiments, results in decreased nitrogen assimilation by the bacterium. Here, the assimilation can be influenced by the excretion rate of ammonia. By targeting the assimilation of ammonia, nitrogen availability can be increased. In some cases influencing ammonia assimilation can be through decreasing the rate of ammonia reuptake after excretion. To decrease the rate of ammonia reuptake after excretion, any relevant gene can be knocked out. An example of an ammonia reuptake gene includes, but is not limited to, amtB. Thus, in some embodiments, the genetically engineered bacterium includes a modification in a nitrogen assimilation genetic network which modification results in decreased expression of amtB.

The nitrogen assimilation control protein (NAC) is a LysR-type transcriptional regulator (LTTR) that is made under conditions of nitrogen-limited growth. NAC can activate the transcription of 670-dependent genes whose products provide the cell with ammonia or glutamate. NAC can also repress genes whose products use ammonia and its own transcription. NAC is encoded by the oxyR gene in K. variicola. In some embodiments, the genetically engineered bacterium's modification is a modification in the oxyR gene such as a modification such as a substitution of the promoter regulating oxyR expression or a deletion of all or a portion of the oxyR gene. In some embodiments, the genetically engineered bacterium's modification in the oxyR gene results in increased ammonia excretion.

In some cases, the assimilation can be influenced by the plant uptake rate. By targeting the plant nitrogen assimilation genes and pathways, nitrogen availability m can ay be increased. In some cases, ammonia assimilation by a plant can be altered through inoculation with N-fixing plant growth promoting microbes. A screen can be carried out to identify microbes which induce ammonia assimilation in plants.

### Increasing Nitrogen Fixation Through Colonization

Increasing the colonization capacity of the microbes can increase the amount of fixed nitrogen provided to a plant. The colonization can be influenced by altering carrying capacity (the abundance of microbes on the root surface) and/or microbe fitness. In some cases, influencing carrying capacity and microbe fitness can be achieved through altering organic acid transport. Organic acid transport can be improved by upregulating relevant genes. An example of an organic acid transport gene includes, but is not limited to, dctA. Other examples of organic acid transport genes include yjjPB, ychM, dauA, and actP.

For example, the colonization capacity can be affected by expression of agglutinins. Increased expression of agglutinins can help the microbes stick to plant roots. Examples of agglutinin genes can include, but are not limited to, fhaB and fhaC.

The colonization capacity can be affected by an increase in endophytic entry. For example, endophytic entry can be affected by plant cell wall-degrading enzymes (CDWE). Increasing CDWE expression and/or secretion can increase the colonization and endophytic entry of the microbes. Some examples of CDWEs include, but are not limited to, polygalacturonases and cellulases. An example of a polygalacturonase gene is pehA. In some cases, export of polygalacturonases and cellulases can be increased by providing an export signal with the enzymes. Other examples of CDWEs include, but are not limited to, xylanases, xyloglucanades, alpha-galactosidases, beta-mannanases, alpha-arabinosidases, beta-galactosidases, and beta-glucuronidases. Exemplary xylanases include yieL1, yieL2, yieL3, yieL4, and pgab. Exemplary alpha-galactosidases include rafA and melA. Exemplary beta-glucuronidases include uidA. Exemplary mannanases include manA. Exemplary alpha-arabinosidases include abfA and abnA. Exemplary beta-galactosidases include lacZ.

Varying the carrying capacity can result in an increased amount of nitrogen being provided to an associated plant. Carrying capacity can be affected by biofilm formation. In some cases, carrying capacity can be affected by small RNA rsmZ. Small RNA rsmZ is a negative regulator of biofilm formation. In some cases, biofilm formation can be promoted by deleting or downregulating rsmZ, leading to increased translation of rsmA (a positive regulator of secondary metabolism) and biofilm formation.

In some cases, biofilm formation can be influenced by enhancing the ability of strains to adhere to the root surface. In some cases, biofilm formation can be promoted by upregulating large adhesion proteins. An example of a large adhesion protein includes, but is not limited to, lapA.

In some cases, carrying capacity can be affected by quorum sensing. In some cases, quorum sensing can be enhanced by increasing the copy number of AHL biosynthesis genes.

In some cases, the colonization of the rhizosphere can be influenced by root mass. For example, root mass can be affected by microbial IAA biosynthesis. Increased IAA biosynthesis by the microbe can stimulate root biomass formation. In some cases, influencing IAA biosynthesis can be achieved through upregulation (at a range of levels) of IAA biosynthesis genes. An example of an IAA biosynthesis gene includes, but is not limited to, ipdC.

In some cases, ethylene signaling can induce systemic resistance in the plant and affect the colonization capacity of the microbe. Ethylene is a plant signaling molecule that elicits a wide range of responses based on plant tissue and ethylene level. The prevailing model for root ethylene response is that plants that are exposed to stress quickly respond by producing a small peak of ethylene that initiates a protective response by the plant, for example, transcription of genes encoding defensive proteins. If the stress persists or is intense, a second much larger peak of ethylene occurs, often several days later. This second ethylene peak induces processes such as senescence, chlorosis, and abscission that can lead to a significant inhibition of plant growth and survival. In some cases, plant growth promoting bacteria can stimulate root growth by producing the auxin IAA, which stimulates a small ethylene response in the roots. At the same time, the bacteria can prevent the second large ethylene peak by producing an enzyme (ACC deaminase) that slows ethylene production in the plant, thus maintaining an ethylene level that's conducive to stimulating root growth. Induction of systemic resistance in the plant can be influenced by bacterial IAAs. In some cases, stimulating IAA biosynthesis can be achieved through upregulation (at a range of levels) of IAA biosynthesis genes. An example of a biosynthesis gene includes, but is not limited to, ipdC.

In some cases, colonization can be affected by ACC Deaminase. ACC Deaminase can be decrease ethylene production in the root by shunting ACC to a side product. In some cases, influencing ACC Deaminase can be achieved through upregulation of ACC Deaminase genes. Some examples of ACC Deaminase genes include, but are not limited to, dcyD.

In some cases, the colonization can be influenced by carrying capacity and/or microbe fitness. For example, carrying capacity and/or microbe fitness can be affected by trehalose overproduction. Trehalose overproduction can increase of drought tolerance. In some cases, influencing trehalose overproduction can be achieved through upregulation (at a range of levels) of trehalose biosynthesis genes. Some examples of trehalose biosynthesis genes include, but are not limited to, otsA, otsB, treZ, and treY. In some cases, upregulation of otsB can also increase nitrogen fixation activity.

In some cases, carrying capacity can be affected by root attachment. Root attachment can be influenced by exopolysaccharide secretion. In some cases, influencing exopolysaccharide secretion can be achieved through upregulation of exopolysaccharide production proteins. Some examples of exopolysaccharide production proteins include, but are not limited to, yjbE and pssM. In some cases, influencing exopolysaccharide secretion may be achieved through upregulation of cellulose biosynthesis. Some examples of cellulose biosynthesis genes include, but are not limited to, acs genes and bcs gene clusters.

In some cases, carrying capacity and/or the microbe's fitness can be affected by fungal inhibition. Fungal inhibition can be influenced by chitinases which can break down fungal cell walls and can lead to biocontrol of rhizosphere fungi. In some cases, influencing fungal inhibition can be achieved through upregulation of chitinase genes. Some examples of chitinase genes include, but are not limited to, chitinase class 1 and chiA.

In some cases, efficient iron uptake can help microbes survive in the rhizosphere where they compete with other soil microbes and the plant for iron uptake. In some cases, high-affinity chelation (siderophores) and transport systems can help with rhizosphere competency by 1) ensuring the microbes obtains enough iron and 2) reducing the iron pool for competing species. Increasing the microbe's ability to do this could increase its competitive fitness in the rhizosphere. In some cases, influencing iron uptake can be by upregulating siderophore genes. Some examples of siderophore genes include, but are not limited to, yhfA, yusV, sbnA, fiu, yfiZ, and fur. Examples of yusV genes include, but are not limited to, yusV1 and yusV2. In some cases iron uptake can be influenced by the tonB transport system. In some cases, influencing iron uptake can be by upregulating tonB transport system genes. Some examples of tonB transport system genes include, but are not limited to, tonB and exbAB.

In some cases, carrying capacity and/or microbe fitness can be affected by redox balance and/or ROS scavenging. Redox balance and/or ROS scavenging can be influenced by bacterial glutathione (GSH) biosynthesis. In some cases, influencing bacterial glutathione (GSH) biosynthesis can be through upregulation of bacterial glutathione biosynthesis genes. Some examples of bacterial glutathione biosynthesis genes include, but are not limited to, gshA, gshAB, and gshB.

In some cases, Redox balance can be influenced by ROS scavenging. In some cases, influencing ROS scavenging can be through upregulation of catalases. Some examples of catalases genes include, but are not limited to, katEG and Mn catalase.

In some cases, biofilm formation can be influenced by phosphorus signaling. In some cases, influencing phosphorus signaling can be through altering the expression of phosphorous signaling genes. Some examples of phosphorous signaling genes include, but are not limited to, phoR and phoB.

In some cases, carrying capacity can be affected by root attachment. Root attachment can be influenced by surfactin biosynthesis. In some cases, influencing surfactin biosynthesis can be achieved by upregulating surfactin biosynthesis to improve biofilm formation. An example of surfactin biosynthesis genes includes, but is not limited to, srfAA.

In some cases, the colonization and/or microbe fitness can be influenced by carrying capacity, competition with other microbes and/or crop protection from other microbes. In some cases, competition with other microbes and/or crop protection from other microbes can be influenced by quorum sensing and/or quorum quenching. Quorum quenching can influence colonization by inhibiting quorum-sensing of potential pathogenic/competing bacteria. In some cases, influencing quorum quenching can be achieved by inserting and/or upregulating genes encoding quorum quenching enzymes. Some examples of quorum quenching genes include, but are not limited to, ahlD, Y2-aiiA, aiiA, ytnP, and attM. In some cases, modification of enzymes involved in quorum quenching, such as Y2-aiiA and/or ytnP can be beneficial for colonization. In some cases, upregulation of Y2-aiiA and/or ytnP can result in hydrolysis of extracellular acyl-homoserine lactone (AHL). aiiA is an N-acyl homoserine lactonase that is an enzyme that breaks down homoserine lactone. Breaking down AHL can stop or slow the quorum signaling ability of competing gram negative bacteria. In some cases, strains that can be utilized in this process of increasing colonization can include, but are not limited to, Rahnella aquatilis, Kosakonia sacchari, and Klebsiella variicola strains.

In some cases, carrying capacity and/or microbe fitness can be affected by rhizobitoxine biosynthesis. Rhizobitoxine biosynthesis can decrease ethylene production in the root by inhibiting ACC synthase. In some cases, influencing rhizobitoxine biosynthesis can be achieved by upregulating rhizobitoxine biosynthesis genes.

In some cases, carrying capacity can be affected by root attachment. Root attachment can be influenced by exopolysaccharide secretion. In some cases, influencing exopolysaccharide secretion can be achieved by generating hypermucoid mutants by deleting mucA.

In some cases, root attachment can be influenced by phenazine biosynthesis. In some cases, influencing phenazine biosynthesis can be achieved by upregulating phenazine biosynthesis genes to improve biofilm formation.

In some cases, root attachment can be influenced by cyclic lipopeptide (CLP) biosynthesis. In some cases, influencing cyclic lipopeptide (CLP) biosynthesis can be achieved by upregulating CLP biosynthesis to improve biofilm formation. In some cases, carrying capacity and/or competition can be affected by antibiotic synthesis. Antibiotic synthesis can increase antibiotic production to kill competing microbes. In some cases, increasing antibiotic production can be achieved by mining genomes for antibiotic biosynthesis pathways and upregulation.

In some cases, colonization can be affected by desiccation tolerance. In some cases, modification of rpoE can be beneficial for colonization. In some cases, upregulation of rpoE can result in increasing expression of stress tolerance genes and pathways. In some cases, rpoE can be upregulated using a unique switchable promoter. In some cases, rpoE can be upregulated using an arabinose promoter. rpoE is a sigma factor similar to phyR. When expressed, rpoE can cause upregulation of multiple stress tolerance genes. As stress tolerance enzymes can not be useful during a colonization cycle, a switchable promoter can be used. In some cases, the promoter can be active during biomass growth and/or during seed coating. In some cases, a switchable promoter can be used where the sugar or chemical can be spiked in during the log phase of biomass growth but can also have the promoter not turned on during one or more other applications of the microbe. In some cases, rpoE can be upregulated while also downregulating rseA. In some cases, strains that can be utilized in this process of increasing colonization can include, but are not limited to, Rahnella aquatilis, Kosakonia sacchari, and Klebsiella variicola strains.

In some cases, colonization can be affected by desiccation tolerance. In some cases, modification of rseA can be beneficial for colonization. In some cases, rseA can be downregulated using a unique switchable promoter. In some cases, rseA can be downregulated using an arabinose promoter. rseA is an anti-sigma factor coexpressed with rpoE. In some cases, the enzymes remain bound to each other, which can decrease or disable rpoE's ability to act as a transcription factor. However, during stress conditions, resA can be cleaved and rpoE can be free to up/down regulate stress tolerance genes. By breaking co-transcription with rpoE, levels of rpoE and resA can be titered independently, which can be beneficial in optimizing colonization of engineered strains. Other gene modifications which can improve desiccation tolerance include modifications in rpoS, treA, treB, phoP, phoQ, and rpoN. In some cases, strains that can be utilized in this process of increasing colonization can include, but are not limited to, Rahnella aquatilis, Kosakonia sacchari, and Klebsiella variicola strains.

Other gene modifications which can improve colonization include modifications in pga, SdiA, fimA1, fimA2, fimA3, fimA4, wzxE, and bolA.

### Generation of Bacterial Populations

### Isolation of Bacteria

Microbes useful in methods and compositions disclosed herein can be obtained by extracting microbes from surfaces or tissues of native plants. Microbes can be obtained by grinding seeds to isolate microbes. Microbes can be obtained by planting seeds in diverse soil samples and recovering microbes from tissues. Additionally, microbes can be obtained by inoculating plants with exogenous microbes and determining which microbes appear in plant tissues. Non-limiting examples of plant tissues can include a seed, seedling, leaf, cutting, plant, bulb, or tuber.

A method of obtaining microbes can be through the isolation of bacteria from soils. Bacteria can be collected from various soil types. In some example, the soil can be characterized by traits such as high or low fertility, levels of moisture, levels of minerals, and various cropping practices. For example, the soil can be involved in a crop rotation where different crops are planted in the same soil in successive planting seasons. The sequential growth of different crops on the same soil can prevent disproportionate depletion of certain minerals. The bacteria can be isolated from the plants growing in the selected soils. The seedling plants can be harvested at 2-6 weeks of growth. For example, at least 400 isolates can be collected in a round of harvest. Soil and plant types reveal the plant phenotype as well as the conditions, which allow for the downstream enrichment of certain phenotypes.

Microbes can be isolated from plant tissues to assess microbial traits. The parameters for processing tissue samples can be varied to isolate different types of associative microbes, such as rhizopheric bacteria, epiphytes, or endophytes. The isolates can be cultured in nitrogen-free media to enrich for bacteria that perform nitrogen fixation. Alternatively, microbes can be obtained from global strain banks.

In planta analytics are performed to assess microbial traits. In some embodiments, the plant tissue can be processed for screening by high throughput processing for DNA and RNA. Additionally, non-invasive measurements can be used to assess plant characteristics, such as colonization. Measurements on wild microbes can be obtained on a plant-by-plant basis. Measurements on wild microbes can also be obtained in the field using medium throughput methods. Measurements can be done successively over time. Model plant system can be used including, but not limited to, Setaria.

Microbes in a plant system can be screened via transcriptional profiling of a microbe in a plant system. Examples of screening through transcriptional profiling are using methods of quantitative polymerase chain reaction (qPCR), molecular barcodes for transcript detection, Next Generation Sequencing, and microbe tagging with fluorescent markers. Impact factors can be measured to assess colonization in the greenhouse including, but not limited to, microbiome, abiotic factors, soil conditions, oxygen, moisture, temperature, inoculum conditions, and root localization. Nitrogen fixation can be assessed in bacteria by measuring ¹⁵N gas/fertilizer (dilution) with IRMS or NanoSIMS as described herein. NanoSIMS is high-resolution secondary ion mass spectrometry. The NanoSIMS technique is a way to investigate chemical activity from biological samples. The catalysis of reduction of oxidation reactions that drive the metabolism of microorganisms can be investigated at the cellular, subcellular, molecular and elemental level. NanoSIMS can provide high spatial resolution of greater than 0.1 μm. NanoSIMS can detect the use of isotope tracers such as ¹³C, ¹⁵N, and ¹⁸O. Therefore, NanoSIMS can be used to the chemical activity nitrogen in the cell.

Automated greenhouses can be used for planta analytics. Plant metrics in response to microbial exposure include, but are not limited to, biomass, chloroplast analysis, CCD camera, volumetric tomography measurements.

One way of enriching a microbe population is according to genotype. For example, a polymerase chain reaction (PCR) assay with a targeted primer or specific primer. Primers designed for the nifH gene can be used to identity diazotrophs because diazotrophs express the nifH gene in the process of nitrogen fixation. A microbial population can also be enriched via single-cell culture-independent approaches and chemotaxis-guided isolation approaches. Alternatively, targeted isolation of microbes can be performed by culturing the microbes on selection media. Premeditated approaches to enriching microbial populations for desired traits can be guided by bioinformatics data and are described herein.

Enriching for Microbes with Nitrogen Fixation Capabilities Using Bioinformatics

Bioinformatic tools can be used to identify and isolate Rhizobacteria, which are selected based on their ability to perform nitrogen fixation. Microbes with high nitrogen fixing ability can promote favorable traits in plants. Bioinformatic modes of analysis for the identification of such Rhizobacteria include, but are not limited to, genomics, metagenomics, targeted isolation, gene sequencing, transcriptome sequencing, and modeling.

Genomics analysis can be used to identify nitrogen fixing Rhizobacteria and confirm the presence of mutations with methods of Next Generation Sequencing as described herein and microbe version control.

Metagenomics can be used to identify and isolate nitrogen fixing Rhizobacteria using a prediction algorithm for colonization. Metadata can also be used to identify the presence of an engineered strain in environmental and greenhouse samples.

Transcriptomic sequencing can be used to predict genotypes leading to nitrogen fixing phenotypes. Additionally, transcriptomic data is used to identify promoters for altering gene expression. Transcriptomic data can be analyzed in conjunction with the Whole Genome Sequence (WGS) to generate models of metabolism and gene regulatory networks.

### Domestication of Microbes

Microbes isolated from nature can undergo a domestication process wherein the microbes are converted to a form that is genetically trackable and identifiable. One way to domesticate a microbe is to engineer it with antibiotic resistance. The process of engineering antibiotic resistance can begin by determining the antibiotic sensitivity in the wild type microbial strain. If the bacteria are sensitive to the antibiotic, then the antibiotic can be a good candidate for antibiotic resistance engineering. Subsequently, an antibiotic resistant gene or a counterselectable suicide vector can be incorporated into the genome of a microbe using recombineering methods. A counterselectable suicide vector can consist of a deletion of the gene of interest, a selectable marker, and the counterselectable marker sacB. Counterselection can be used to exchange native microbial DNA sequences with antibiotic resistant genes. A medium throughput method can be used to evaluate multiple microbes simultaneously allowing for parallel domestication. Alternative methods of domestication include the use of homing nucleases to prevent the suicide vector sequences from looping out or from obtaining intervening vector sequences.

DNA vectors can be introduced into bacteria via several methods including electroporation and chemical transformations. A standard library of vectors can be used for transformations. An example of a method of gene editing is CRISPR preceded by Cas9 testing to ensure activity of Cas9 in the microbes.

### Non-transgenic Engineering of Microbes

A microbial population with favorable traits can be obtained via directed evolution. Direct evolution is an approach wherein the process of natural selection is mimicked to evolve proteins or nucleic acids towards a user-defined goal. An example of direct evolution is when random mutations are introduced into a microbial population, the microbes with the most favorable traits are selected, and the growth of the selected microbes is continued. The most favorable traits in Rhizobacteria can be in nitrogen fixation. The method of directed evolution can be iterative and adaptive based on the selection process after each iteration.

Rhizobacteria with high capability of nitrogen fixation can be generated. The evolution of these Rhizobacteria can be carried out via the introduction of genetic modification. Genetic modification can be introduced via polymerase chain reaction mutagenesis, oligonucleotide-directed mutagenesis, saturation mutagenesis, fragment shuffling mutagenesis, homologous recombination, CRISPR/Cas9 systems, chemical mutagenesis, and combinations thereof. These approaches can introduce random mutations into the microbial population. For example, mutants can be generated using synthetic DNA or RNA via oligonucleotide-directed mutagenesis. Mutants can be generated using tools contained on plasmids, which are later cured. Genes of interest can be identified using libraries from other species with improved traits including, but not limited to, improved nitrogen fixing properties, improved colonization of cereals, increased oxygen sensitivity, increased nitrogen fixation, and increased ammonia excretion. Intrageneric genes can be designed based on these libraries using software such as Geneious or Platypus design software. Mutations can be designed with the aid of machine learning. Mutations can be designed with the aid of a metabolic model. Automated design of the mutation can be done using a la Platypus and will guide RNAs for Cas-directed mutagenesis.

The intra-generic genes can be transferred into the host microbe. Additionally, reporter systems can also be transferred to the microbe. The reporter systems characterize promoters, determine the transformation success, screen mutants, and act as negative screening tools.

The microbes carrying the mutation can be cultured via serial passaging. A microbial colony contains a single variant of the microbe. Microbial colonies are screened with the aid of an automated colony picker and liquid handler. Mutants with gene duplication and increased copy number express a higher genotype of the desired trait.

### Selection of Plant Growth Promoting Microbes Based on Nitrogen Fixation

The microbial colonies can be screened using various assays to assess nitrogen fixation. One way to measure nitrogen fixation is via a single fermentative assay, which measures nitrogen excretion. An alternative method is the acetylene reduction assay (ARA) with in-line sampling over time. ARA can be performed in high throughput plates of microtube arrays. ARA can be performed with live plants and plant tissues. The media formulation and media oxygen concentration can be varied in ARA assays. Another method of screening microbial variants is by using biosensors. The use of NanoSIMS and Raman microspectroscopy can be used to investigate the activity of the microbes. In some cases, bacteria can also be cultured and expanded using methods of fermentation in bioreactors. The bioreactors are designed to improve robustness of bacteria growth and to decrease the sensitivity of bacteria to oxygen. Medium to high TP plate-based microfermentors are used to evaluate oxygen sensitivity, nutritional needs, nitrogen fixation, and nitrogen excretion. The bacteria can also be co-cultured with competitive or beneficial microbes to elucidate cryptic pathways. Flow cytometry can be used to screen for bacteria that produce high levels of nitrogen using chemical, colorimetric, or fluorescent indicators. The bacteria can be cultured in the presence or absence of a nitrogen source. For example, the bacteria can be cultured with glutamine, ammonia, urea or nitrates.

### Microbe Breeding

Microbe breeding is a method to systematically identify and improve the role of species within the crop microbiome. The method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters, and 3) screening and selection of new microbial genotypes that produce desired crop phenotypes. To systematically assess the improvement of strains, a model is created that links colonization dynamics of the microbial community to genetic activity by key species. The model is used to predict genetic targets breeding and improve the frequency of selecting improvements in microbiome-encoded traits of agronomic relevance.

Production of bacteria to improve plant traits (e.g., nitrogen fixation) can be achieved through serial passage. The production of this bacteria can be done by selecting plants, which have a particular improved trait that is influenced by the microbial flora, in addition to identifying bacteria and/or compositions that are capable of imparting one or more improved traits to one or more plants. One method of producing a bacteria to improve a plant trait includes the steps of: (a) isolating bacteria from tissue or soil of a first plant; (b) introducing a genetic modification into one or more of the bacteria to produce one or more variant bacteria; (c) exposing a plurality of plants to the variant bacteria; (d) isolating bacteria from tissue or soil of one of the plurality of plants, wherein the plant from which the bacteria is isolated has an improved trait relative to other plants in the plurality of plants; and (e) repeating steps (b) to (d) with bacteria isolated from the plant with an improved trait (step (d)). Steps (b) to (d) can be repeated any number of times (e.g., once, twice, three times, four times, five times, ten times, or more) until the improved trait in a plant reaches a desired level. Further, the plurality of plants can be more than two plants, such as about 10 to about 20 plants, or about 20 or more, about 50 or more, about 100 or more, about 300 or more, about 500 or more, or about 1000 or more plants.

In addition to obtaining a plant with an improved trait, a bacterial population comprising bacteria comprising one or more genetic modifications introduced into one or more genes (e.g., genes regulating nitrogen fixation) is obtained. By repeating the steps described above, a population of bacteria can be obtained that include the most appropriate members of the population that correlate with a plant trait of interest. The bacteria in this population can be identified and their beneficial properties determined, such as by genetic and/or phenotypic analysis. Genetic analysis can occur of isolated bacteria in step (a). Phenotypic and/or genotypic information can be obtained using techniques including: high through-put screening of chemical components of plant origin, sequencing techniques including high throughput sequencing of genetic material, differential display techniques (including DDRT-PCR, and DD-PCR), nucleic acid microarray techniques, RNA-sequencing (Whole Transcriptome Shotgun Sequencing), and qRT-PCR (quantitative real time PCR). Information gained can be used to obtain community profiling information on the identity and activity of bacteria present, such as phylogenetic analysis or microarray-based screening of nucleic acids coding for components of rRNA operons or other taxonomically informative loci. Examples of taxonomically informative loci include 16S rRNA gene, 23S rRNA gene, 5S rRNA gene, 5.8S rRNA gene, 12S rRNA gene, 18S rRNA gene, 28S rRNA gene, gyrB gene, rpoB gene, fusA gene, recA gene, coxl gene, nifD gene. Example processes of taxonomic profiling to determine taxa present in a population are described in US20140155283. Bacterial identification can comprise characterizing activity of one or more genes or one or more signaling pathways, such as genes associated with the nitrogen fixation pathway. Synergistic interactions (where two components, by virtue of their combination, increase a desired effect by more than an additive amount) between different bacterial species can also be present in the bacterial populations.

The genetic modification can be a gene selected from the group consisting of: nifA, nifL, ntrB, ntrC, glnA, glnB, glnK, draT, amtB, glnD, glnE, nifJ, nifH, nifD, nifK, nifY, nifE, nifN, nifU, nifS, nifV, nifW, nifZ, nifM, nifF, nifB, and nifQ. The genetic modification can be a modification in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, or NAD+-dinitrogen-reductase aDP-D-ribosyltransferase. The genetic modification can be a mutation that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD. Introducing a genetic modification can comprise insertion and/or deletion of one or more nucleotides at a target site, such as 1, 2, 3, 4, 5, 10, 25, 50, 100, 250, 500, or more nucleotides. The genetic modification introduced into one or more bacteria of the methods disclosed herein can be a knock-out mutation (e.g. deletion of a promoter, insertion or deletion to produce a premature stop codon, deletion of an entire gene), or it can be elimination or abolishment of activity of a protein domain (e.g. point mutation affecting an active site, or deletion of a portion of a gene encoding the relevant portion of the protein product), or it can alter or abolish a regulatory sequence of a target gene. One or more regulatory sequences can also be inserted, including heterologous regulatory sequences and regulatory sequences found within a genome of a bacterial species or genus corresponding to the bacteria into which the genetic modification is introduced. Moreover, regulatory sequences can be selected based on the expression level of a gene in a bacterial culture or within a plant tissue. The genetic modification can be a pre-determined genetic modification that is specifically introduced to a target site. The genetic modification can be a random mutation within the target site. The genetic modification can be an insertion or deletion of one or more nucleotides. In some cases, a plurality of different genetic modifications (e.g. 2, 3, 4, 5, 10, or more) are introduced into one or more of the isolated bacteria before exposing the bacteria to plants for assessing trait improvement. The plurality of genetic modifications can be any of the above types, the same or different types, and in any combination. In some cases, a plurality of different genetic modifications are introduced serially, introducing a first genetic modification after a first isolation step, a second genetic modification after a second isolation step, and so forth so as to accumulate a plurality of genetic modifications in bacteria imparting progressively improved traits on the associated plants.

A variety of molecular tools and methods are available for introducing genetic modification. For example, genetic modification can be introduced via polymerase chain reaction mutagenesis, oligonucleotide-directed mutagenesis, saturation mutagenesis, fragment shuffling mutagenesis, homologous recombination, recombineering, lambda red mediated recombination, CRISPR/Cas9 systems, chemical mutagenesis, and combinations thereof. Chemical methods of introducing genetic modification include exposure of DNA to a chemical mutagen, e.g., ethyl methanesulfonate (EMS), methyl methanesulfonate (MMS), N-nitrosourea (EN U), N-methyl-N-nitro-N′-nitrosoguanidine, 4-nitroquinoline N-oxide, diethylsulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz(a)anthracene, chlorambucil, hexamethylphosphoramide, bisulfan, and the like. Radiation mutation-inducing agents include ultraviolet radiation, γ-irradiation, X-rays, and fast neutron bombardment. Genetic modification can also be introduced into a nucleic acid using, e.g., trimethylpsoralen with ultraviolet light. Random or targeted insertion of a mobile DNA element, e.g., a transposable element, is another suitable method for generating genetic modification. Genetic modifications can be introduced into a nucleic acid during amplification in a cell-free in vitro system, e.g., using a polymerase chain reaction (PCR) technique such as error-prone PCR. Genetic modifications can be introduced into a nucleic acid in vitro using DNA shuffling techniques (e.g., exon shuffling, domain swapping, and the like). Genetic modifications can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Example descriptions of various methods for introducing genetic modifications are provided in e.g., Stemple (2004) Nature 5:1-7; Chiang et al. (1993) PCR Methods Appl 2(3): 210-217; Stemmer (1994) Proc. Natl. Acad. Sci. USA 91:10747-10751; and U.S. Pat. Nos. 6,033,861, and 6,773,900.

Genetic modifications introduced into microbes can be classified as transgenic, cisgenic, intragenomic, intrageneric, intergeneric, synthetic, evolved, rearranged, or SNPs.

Genetic modification can be introduced into numerous metabolic pathways within microbes to elicit improvements in the traits described above. Representative pathways include sulfur uptake pathways, glycogen biosynthesis, the glutamine regulation pathway, the molybdenum uptake pathway, the nitrogen fixation pathway, ammonia assimilation, ammonia excretion or secretion, Nitrogen uptake, glutamine biosynthesis, annamox, phosphate solubilization, organic acid transport, organic acid production, agglutinins production, reactive oxygen radical scavenging genes, Indole Acetic Acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signaling genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production. CRISPR/Cas9 (Clustered regularly interspaced short palindromic repeats)/CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently link to form a single molecule (also called a single guide RNA (“sgRNA”). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Further exemplary descriptions of CRISPR systems for introducing genetic modification can be found in, e.g. U.S. Pat. No. 8,795,965.

As a cyclic amplification technique, polymerase chain reaction (PCR) mutagenesis uses mutagenic primers to introduce desired mutations. PCR is performed by cycles of denaturation, annealing, and extension. After amplification by PCR, selection of mutated DNA and removal of parental plasmid DNA can be accomplished by: 1) replacement of dCTP by hydroxymethylated-dCTP during PCR, followed by digestion with restriction enzymes to remove non-hydroxymethylated parent DNA only; 2) simultaneous mutagenesis of both an antibiotic resistance gene and the studied gene changing the plasmid to a different antibiotic resistance, the new antibiotic resistance facilitating the selection of the desired mutation thereafter; 3) after introducing a desired mutation, digestion of the parent methylated template DNA by restriction enzyme Dpnl which cleaves only methylated DNA, by which the mutagenized unmethylated chains are recovered; or 4) circularization of the mutated PCR products in an additional ligation reaction to increase the transformation efficiency of mutated DNA. Further description of exemplary methods can be found in, e.g., U.S. Pat. Nos. 7,132,265, 6,713,285, 6,673,610, 6,391,548, 5,789,166, 5,780,270, 5,354,670, 5,071,743, and U.S. Publication No. 2010/0267147.

Oligonucleotide-directed mutagenesis, also called site-directed mutagenesis, typically utilizes a synthetic DNA primer. This synthetic primer contains the desired mutation and is complementary to the template DNA around the mutation site so that it can hybridize with the DNA in the gene of interest. The mutation can be a single base change (a point mutation), multiple base changes, deletion, or insertion, or a combination of these. The single-strand primer is then extended using a DNA polymerase, which copies the rest of the gene. The gene thus copied contains the mutated site, and can then be introduced into a host cell as a vector and cloned. Finally, mutants can be selected by DNA sequencing to check that they contain the desired mutation.

Genetic modifications can be introduced using error-prone PCR. In this technique the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template. Another means of introducing random mutations is exposing cells to a chemical mutagen, such as nitrosoguanidine or ethyl methanesulfonate (Nestmann, Mutat Res 1975 June; 28(3):323-30), and the vector containing the gene is then isolated from the host.

Saturation mutagenesis is another form of random mutagenesis, in which one tries to generate all or nearly all possible mutations at a specific site, or narrow region of a gene. In a general sense, saturation mutagenesis is comprised of mutagenizing a complete set of mutagenic cassettes (wherein each cassette is, for example, 1-500 bases in length) in defined polynucleotide sequence to be mutagenized (wherein the sequence to be mutagenized is, for example, from 15 to 100,000 bases in length). Therefore, a group of mutations (e.g. ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons, and groupings of particular nucleotide cassettes.

Fragment shuffling mutagenesis, also called DNA shuffling, is a way to rapidly propagate beneficial mutations. In an example of a shuffling process, DNAse is used to fragment a set of parent genes into pieces of e.g. about 50-100 bp in length. This is then followed by a polymerase chain reaction (PCR) without primers—DNA fragments with sufficient overlapping homologous sequence will anneal to each other and are then be extended by DNA polymerase. Several rounds of this PCR extension are allowed to occur, after some of the DNA molecules reach the size of the parental genes. These genes can then be amplified with another PCR, this time with the addition of primers that are designed to complement the ends of the strands. The primers can have additional sequences added to their 5′ ends, such as sequences for restriction enzyme recognition sites needed for ligation into a cloning vector. Further examples of shuffling techniques are provided in U.S. Publication No. 2005/0266541.

Homologous recombination mutagenesis involves recombination between an exogenous DNA fragment and the targeted polynucleotide sequence. After a double-stranded break occurs, sections of DNA around the 5′ ends of the break are cut away in a process called resection. In the strand invasion step that follows, an overhanging 3′ end of the broken DNA molecule then “invades” a similar or identical DNA molecule that is not broken. The method can be used to delete a gene, remove exons, add a gene, and introduce point mutations. Homologous recombination mutagenesis can be permanent or conditional. Typically, a recombination template is also provided. A recombination template can be a component of another vector, contained in a separate vector, or provided as a separate polynucleotide. In some embodiments, a recombination template is designed to serve as a template in homologous recombination, such as within or near a target sequence nicked or cleaved by a site-specific nuclease. A template polynucleotide can be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. Non-limiting examples of site-directed nucleases useful in methods of homologous recombination include zinc finger nucleases, CRISPR nucleases, TALE nucleases, and meganuclease. For a further description of the use of such nucleases, see e.g. U.S. Pat. No. 8,795,965 and U.S. Publication No. 2014/0301990.

Mutagens that create primarily point mutations and short deletions, insertions, transversions, and/or transitions, including chemical mutagens or radiation, can be used to create genetic modifications. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Introducing genetic modification can be an incomplete process, such that some bacteria in a treated population of bacteria carry a desired mutation while others do not. In some cases, it is desirable to apply a selection pressure so as to enrich for bacteria carrying a desired genetic modification. Traditionally, selection for successful genetic variants involved selection for or against some functionality imparted or abolished by the genetic modification, such as in the case of inserting antibiotic resistance gene or abolishing a metabolic activity capable of converting a non-lethal compound into a lethal metabolite. It is also possible to apply a selection pressure based on a polynucleotide sequence itself, such that only a desired genetic modification need be introduced (e.g. without also requiring a selectable marker). In this case, the selection pressure can comprise cleaving genomes lacking the genetic modification introduced to a target site, such that selection is effectively directed against the reference sequence into which the genetic modification is sought to be introduced. Typically, cleavage occurs within 100 nucleotides of the target site (e.g. within 75, 50, 25, 10, or fewer nucleotides from the target site, including cleavage at or within the target site). Cleaving can be directed by a site-specific nuclease selected from the group consisting of a Zinc Finger nuclease, a CRISPR nuclease, a TALE nuclease (TALEN), or a meganuclease. Such a process is similar to processes for enhancing homologous recombination at a target site, except that no template for homologous recombination is provided. As a result, bacteria lacking the desired genetic modification are more likely to undergo cleavage that, left unrepaired, results in cell death. Bacteria surviving selection can then be isolated for use in exposing to plants for assessing conferral of an improved trait.

A CRISPR nuclease can be used as the site-specific nuclease to direct cleavage to a target site. An improved selection of mutated microbes can be obtained by using Cas9 to kill non-mutated cells. Plants are then inoculated with the mutated microbes to re-confirm symbiosis and create evolutionary pressure to select for efficient symbionts. Microbes can then be re-isolated from plant tissues. CRISPR nuclease systems employed for selection against non-variants can employ similar elements to those described above with respect to introducing genetic modification, except that no template for homologous recombination is provided. Cleavage directed to the target site thus enhances death of affected cells.

Other options for specifically inducing cleavage at a target site are available, such as zinc finger nucleases, TALE nuclease (TALEN) systems, and meganuclease. Zinc-finger nucleases (ZFNs) are artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell, ZFNs can be used to edit target DNA in the cell (e.g., the cell's genome) by inducing double stranded breaks. Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell's genome) by inducing double strand breaks. Meganucleases (homing endonuclease) are endodeoxyribonucleases characterized by a large recognition site (double-stranded DNA sequences of 12 to 40 base pairs. Meganucleases can be used to replace, eliminate or modify sequences in a highly targeted way. By modifying their recognition sequence through protein engineering, the targeted sequence can be changed. Meganucleases can be used to modify all genome types, whether bacterial, plant or animal and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

Methods of the present disclosure can be employed to introduce or improve one or more of a variety of desirable traits. Examples of traits that can introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions.

In some embodiments, a trait to be introduced or improved upon is nitrogen fixation, as described herein. In some embodiments, the trait to be introduced or improved upon is ammonium excretion, colonization, iron transport, oxygen tolerance, desiccation tolerance, or a combination thereof. In some embodiments, one or more of ammonium excretion is increased, colonization is increased, iron transport is increased, oxygen tolerance is increased, and desiccation tolerance is increased relative to bacteria lacking modifications in the identified genes. In some cases, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil lacking the introduced or improved upon trait. For example, a plant resulting from the methods described herein can exhibit an increase in the amount of nitrogen fixation that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under the same conditions in the soil. In additional examples, a plant resulting from the methods described herein exhibits a difference in the trait that is at least about 5% greater, for example at least about 5%, at least about 8%, at least about 10%, at least about 15%, at least about 20%, at least about 25%, at least about 30%, at least about 40%, at least about 50%, at least about 60%, at least about 75%, at least about 80%, at least about 80%, at least about 90%, or at least 100%, at least about 200%, at least about 300%, at least about 400% or greater than a reference agricultural plant grown under similar conditions in the soil. In some embodiments, the amount of nitrogen fixation and/or increase in one or more of ammonium excretion, colonization, iron transport, oxygen tolerance, and desiccation tolerance compared to a reference agricultural plant grown under similar conditions in the soil that occurs in the plants described herein is measured using an assay as described herein. For example, the amount of nitrogen fixation can be measured by an acetylene-reduction (AR) assay.

The trait to be improved can be assessed under conditions including the application of one or more biotic or abiotic stressors. Examples of stressors include abiotic stresses (such as heat stress, salt stress, drought stress, cold stress, and low nutrient stress) and biotic stresses (such as nematode stress, insect herbivory stress, fungal pathogen stress, bacterial pathogen stress, and viral pathogen stress).

The trait improved by methods and compositions of the present disclosure can be nitrogen fixation, including in a plant not previously capable of nitrogen fixation. In some cases, bacteria isolated according to a method described herein produce 1% or more (e.g. 2%, 3%, 4%, 5%, 6%, 7%, 8%, 9%, 10%, 15%, 20%, or more) of a plant's nitrogen, which can represent an increase in nitrogen fixation capability of at least 2-fold (e.g. 3-fold, 4-fold, 5-fold, 6-fold, 7-fold, 8-fold, 9-fold, 10-fold, 20-fold, 50-fold, 100-fold, 1000-fold, or more) as compared to bacteria isolated from the first plant before introducing any genetic modification. In some cases, the bacteria produce 5% or more of a plant's nitrogen. The desired level of nitrogen fixation can be achieved after repeating the steps of introducing genetic modification, exposure to a plurality of plants, and isolating bacteria from plants with an improved trait one or more times (e.g. 1, 2, 3, 4, 5, 10, 15, 25, or more times). In some cases, enhanced levels of nitrogen fixation are achieved in the presence of fertilizer supplemented with glutamine, ammonia, or other chemical source of nitrogen. Methods for assessing degree of nitrogen fixation are known, examples of which are described herein.

Microbe breeding is a method to systematically identify and improve the role of species within the crop microbiome. The method comprises three steps: 1) selection of candidate species by mapping plant-microbe interactions and predicting regulatory networks linked to a particular phenotype, 2) pragmatic and predictable improvement of microbial phenotypes through intra-species crossing of regulatory networks and gene clusters, and 3) screening and selection of new microbial genotypes that produce desired crop phenotypes. To systematically assess the improvement of strains, a model is created that links colonization dynamics of the microbial community to genetic activity by key species. The model is used to predict genetic targets breeding and improve the frequency of selecting improvements in microbiome-encoded traits of agronomic relevance.

### Nitrogen Fixation

The genetically engineered bacterium disclosed herein can be used in a method of increasing nitrogen fixation in a plant, which method includes a step of contacting the plant with a plurality of the bacterium. In some embodiments, the bacteria produce 1% or more of nitrogen in the plant (e.g. 2%, 5%, 10%, or more), which, in some embodiments, represents a nitrogen-fixation capability of at least 2-fold as compared to the plant in the absence of the bacteria. The bacteria can produce the nitrogen in the presence of fertilizer supplemented with glutamine, urea, nitrates or ammonia. The genetically engineered bacterium can include any genetic modification described herein, including examples provided above, in any number and any combination. The genetic modification can be introduced into a gene selected from the group consisting of nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifU, nifS, nifV, nifW, nifZ, nifM, nifF, nifB, and nifQ. The genetic modification can be a mutation that results in one or more of: increased expression or activity of nifA or glutaminase; decreased expression or activity of nifL, ntrB, glutamine synthetase, glnB, glnK, draT, amtB; decreased adenylyl-removing activity of GlnE; or decreased uridylyl-removing activity of GlnD. The genetic modification introduced into one or more bacteria of the methods disclosed herein can be a knock-out mutation or it can abolish a regulatory sequence of a target gene, or it can comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. The genetic modification can be produced by chemical mutagenesis. The plant can be exposed to biotic or abiotic stressors.

The amount of nitrogen fixation that occurs in the plants described herein can be measured in several ways, for example by an acetylene-reduction (AR) assay. An acetylene-reduction assay can be performed in vitro or in vivo. Evidence that a particular bacterium is providing fixed nitrogen to a plant can include: 1) total plant N significantly increases upon inoculation, preferably with a concomitant increase in N concentration in the plant; 2) nitrogen deficiency symptoms are relieved under N-limiting conditions upon inoculation (which should include an increase in dry matter); 3) N₂ fixation is documented through the use of an ¹⁵N approach (which can be isotope dilution experiments, ¹⁵N₂ reduction assays, or ¹⁵N natural abundance assays); 4) fixed N is incorporated into a plant protein or metabolite; and 5) all of these effects are not be seen in non-inoculated plants or in plants inoculated with a mutant of the inoculum strain.

The wild-type nitrogen fixation regulatory cascade can be represented as a digital logic circuit where the inputs O₂ and NH₄⁺ pass through a NOR gate, the output of which enters an AND gate in addition to ATP. In some embodiments, the methods disclosed herein disrupt the influence of NH₄⁺ on this circuit, at multiple points in the regulatory cascade, so that microbes can produce nitrogen even in fertilized fields. However, the methods disclosed herein also envision altering the impact of ATP or O₂ on the circuitry, or replacing the circuitry with other regulatory cascades in the cell, or altering genetic circuits other than nitrogen fixation. Gene clusters can be re-engineered to generate functional products under the control of a heterologous regulatory system. By eliminating native regulatory elements outside of, and within, coding sequences of gene clusters, and replacing them with alternative regulatory systems, the functional products of complex genetic operons and other gene clusters can be controlled and/or moved to heterologous cells, including cells of different species other than the species from which the native genes were derived. Once re-engineered, the synthetic gene clusters can be controlled by genetic circuits or other inducible regulatory systems, thereby controlling the products' expression as desired. The expression cassettes can be designed to act as logic gates, pulse generators, oscillators, switches, or memory devices. The controlling expression cassette can be linked to a promoter such that the expression cassette functions as an environmental sensor, such as an oxygen, temperature, touch, osmotic stress, membrane stress, or redox sensor.

As an example, the nifL, nifA, nifT, and nifX genes can be eliminated from the nif gene cluster. Synthetic genes can be designed by codon randomizing the DNA encoding each amino acid sequence. Codon selection is performed, specifying that codon usage be as divergent as possible from the codon usage in the native gene. Proposed sequences are scanned for any undesired features, such as restriction enzyme recognition sites, transposon recognition sites, repetitive sequences, sigma 54 and sigma 70 promoters, cryptic ribosome binding sites, and rho independent terminators. Synthetic ribosome binding sites are chosen to match the strength of each corresponding native ribosome binding site, such as by constructing a fluorescent reporter plasmid in which the 150 bp surrounding a gene's start codon (from −60 to +90) is fused to a fluorescent gene. This chimera can be expressed under control of the Ptac promoter, and fluorescence measured via flow cytometry. To generate synthetic ribosome binding sites, a library of reporter plasmids using 150 bp (−60 to +90) of a synthetic expression cassette is generated. Briefly, a synthetic expression cassette can consist of a random DNA spacer, a degenerate sequence encoding an RBS library, and the coding sequence for each synthetic gene. Multiple clones are screened to identify the synthetic ribosome binding site that best matched the native ribosome binding site. Synthetic operons that consist of the same genes as the native operons are thus constructed and tested for functional complementation. A further exemplary description of synthetic operons is provided in US20140329326.

### Bacterial Species

Microbes useful in the methods and compositions disclosed herein can be obtained from any source. In some cases, microbes can be bacteria, archaea, protozoa or fungi. The microbes of this disclosure can be nitrogen fixing microbes, for example a nitrogen fixing bacteria, nitrogen fixing archaea, nitrogen fixing fungi, nitrogen fixing yeast, or nitrogen fixing protozoa. Microbes useful in the methods and compositions disclosed herein can be spore forming microbes, for example spore forming bacteria. In some cases, bacteria useful in the methods and compositions disclosed herein can be Gram positive bacteria or Gram negative bacteria. In some cases, the bacteria can be an endospore forming bacteria of the Firmicute phylum. In some cases, the bacteria can be a diazatroph. In some cases, the bacteria can not be a diazotroph.

The methods and compositions of this disclosure can be used with an archaea, such as, for example, Methanothermobacter thermoautotrophicus.

In some cases, bacteria which can be useful include, but are not limited to, Agrobacterium radiobacter. Bacillus acidocaldarius. Bacillus acidoterrestris. Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolvticus, Bacillus pumilus, Bacillus siamensis, Bacillus smithit, Bacillus sphaericus, Bacillus subtilis, Bacillus thuringiensis, Bacillus uniflagellatus, Bradyrhizobium japonicum, Brevibacillus brevis Brevibacillus laterosporus (formerly Bacillus laterosporus), Chromobacterium subtsugae, Delftia acidovorans, Lactobacillus acidophilus, Lysobacter antibioticus, Lysobacter enzvmogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carolovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradi, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luninescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some cases the bacterium can be Azotobacter chroococcum, Methanosarcina barkeri, Klesiella pneumoniae, Azotobacter vinelandii, Rhodobacter spharoides, Rhodobacter capsulatus, Rhodobcter palustris, Rhodosporillum rubrum, Rhizobium leguminosarum or Rhizobium etli.

In some cases the bacterium can be a species of Clostridium, for example Clostridium pasteurianum. Clostridium betyerinckii. Clostridium perfringens. Clostridium tetani. Clostridium acetobutylicum.

In some cases, bacteria used with the methods and compositions of the present disclosure can be cyanobacteria. Examples of cyanobacterial genuses include Anabaena (for example Anagaena sp. PCC7120), Nostoc (for example Nostoc punctiforme), or Synechocystis (for example Synechocystis sp. PCC6803).

In some cases, bacteria used with the methods and compositions of the present disclosure can belong to the phylum Chlorobi, for example Chlorobium tepidum.

In some cases, microbes used with the methods and compositions of the present disclosure can comprise a gene homologous to a known NifH gene. Sequences of known NifH genes can be found in, for example, the Zehr lab NifH database, (zehr.pmc.ucsc.edu/nifH_Database_Public/ on the World Wide Web, Apr. 4, 2014), or the Buckley lab NifH database (css.cornell.edu/faculty/buckley/nifh.htm on the World Wide Web, and Gaby, John Christian, and Daniel H. Buckley. “A comprehensive aligned nifH gene database: a multipurpose tool for studies of nitrogen-fixing bacteria.” Database 2014 (2014): bau001.). In some cases, microbes used with the methods and compositions of the present disclosure can comprise a sequence which encodes a polypeptide with at least 60%, 70%, 80%, 85%, 90%, 95%, 96%, 96%, 98%, 99% or more than 99% sequence identity to a sequence from the Zehr lab NifH database, (zehr.pmc.ucsc.edu/nifH_Database_Public/ on the World Wide Web, Apr. 4, 2014). In some cases, microbes used with the methods and compositions of the present disclosure can comprise a sequence which encodes a polypeptide with at least 60%, 70%, 80%, 85%, 90%, 95%, 96%, 96%, 98%, 99% or more than 99% sequence identity to a sequence from the Buckley lab NifH database, (Gaby, John Christian, and Daniel H. Buckley. “A comprehensive aligned nifH gene database: a multipurpose tool for studies of nitrogen-fixing bacteria.” Database 2014 (2014): bau001.).

Microbes useful in the methods and compositions disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. Non-limiting examples of plant tissues include a seed, seedling, leaf, cutting, plant, bulb or tuber. In some cases, bacteria are isolated from a seed. The parameters for processing samples can be varied to isolate different types of associative microbes, such as rhizospheric, epiphytes, or endophytes. Bacteria can also be sourced from a repository, such as environmental strain collections, instead of initially isolating from a first plant. The microbes can be genotyped and phenotyped, via sequencing the genomes of isolated microbes; profiling the composition of communities in planta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these.

The bacteria and methods of producing bacteria described herein can apply to bacteria able to self-propagate efficiently on the leaf surface, root surface, or inside plant tissues without inducing a damaging plant defense reaction, or bacteria that are resistant to plant defense responses. The bacteria described herein can be isolated by culturing a plant tissue extract or leaf surface wash in a medium with no added nitrogen. However, the bacteria can be unculturable, that is, not known to be culturable or difficult to culture using standard methods known in the art. The bacteria described herein can be an endophyte or an epiphyte or a bacterium inhabiting the plant rhizosphere (rhizospheric bacteria). The bacteria obtained after repeating the steps of introducing genetic modification, exposure to a plurality of plants, and isolating bacteria from plants with an improved trait one or more times (e.g. 1, 2, 3, 4, 5, 10, 15, 25, or more times) can be endophytic, epiphytic, or rhizospheric. Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some cases, a seed-borne bacterial endophyte is capable of replicating within the plant tissue, for example, the interior of the seed. Also, in some cases, the seed-borne bacterial endophyte is capable of surviving desiccation.

The bacterial isolated according to methods of the disclosure, or used in methods or compositions of the disclosure, can comprise a plurality of different bacterial taxa in combination. By way of example, the bacteria can include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium and Halomonas), Firmicutes (such as Bacillus, Paenibacillus, Lactobacillus, Mycoplasma, and Acetabacterium), and Actinobacteria (such as Streptomyces, Rhodacoccus, Microbacterium, and Curtobacterium). The bacteria used in methods and compositions of this disclosure can include nitrogen fixing bacterial consortia of two or more species. In some cases, one or more bacterial species of the bacterial consortia can be capable of fixing nitrogen. In some cases, one or more species of the bacterial consortia can facilitate or enhance the ability of other bacteria to fix nitrogen. The bacteria which fix nitrogen and the bacteria which enhance the ability of other bacteria to fix nitrogen can be the same or different. In some examples, a bacterial strain can be able to fix nitrogen when in combination with a different bacterial strain, or in a certain bacterial consortia, but can be unable to fix nitrogen in a monoculture. Examples of bacterial genuses which can be found in a nitrogen fixing bacterial consortia include, but are not limited to, Herbaspirillum, Azospirillum, Enterobacter, and Bacillus.

Bacteria that can be produced by the methods disclosed herein include Azotobacter sp., Bradyrhizobium sp., Klebsiella sp., and Sinorhizobium sp. In some cases, the bacteria can be selected from the group consisting of: Azotobacter vinelandii, Bradyrhizobium japonicum, Klebsiella pneumoniae, and Sinorhizobium meliloti. In some cases, the bacteria can be of the genus Enterobacter or Rahnella. In some cases, the bacteria can be of the genus Frankia, or Clostridium. Examples of bacteria of the genus Clostridium include, but are not limited to, Clostridium acetobutilicum, Clostridium pasteurianum, Clostridium beijerinckii, Clostridium perfringens, and Clostridium tetani. In some cases, the bacteria can be of the genus Paenibacillus, for example Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some examples, bacteria isolated according to methods of the disclosure can be a member of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, 25 Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

The bacteria can be obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, and road surfaces).

The plants from which the bacteria are obtained can be a plant having one or more desirable traits, for example a plant which naturally grows in a particular environment or under certain conditions of interest. By way of example, a certain plant can naturally grow in sandy soil or sand of high salinity, or under extreme temperatures, or with little water, or it can be resistant to certain pests or disease present in the environment, and it can be desirable for a commercial crop to be grown in such conditions, particularly if they are, for example, the only conditions available in a particular geographic location. By way of further example, the bacteria can be collected from commercial crops grown in such environments, or more specifically from individual crop plants best displaying a trait of interest amongst a crop grown in any specific environment: for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste or smell. The bacteria can be collected from a plant of interest or any material occurring in the environment of interest, including fungi and other animal and plant biota, soil, water, sediments, and other elements of the environment as referred to previously.

The bacteria can be isolated from plant tissue. This isolation can occur from any appropriate tissue in the plant, including for example root, stem and leaves, and plant reproductive tissues. By way of example, conventional methods for isolation from plants typically include the sterile excision of the plant material of interest (e.g. root or stem lengths, leaves), surface sterilization with an appropriate solution (e.g. 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which can or can not be selective (e.g. contain only phytic acid as a source of phosphorus). This approach is especially useful for bacteria which form isolated colonies and can be picked off individually to separate plates of nutrient medium, and further purified to a single species by well-known methods. Alternatively, the plant root or foliage samples can not be surface sterilized but only washed gently thus including surface-dwelling epiphytic microorganisms in the isolation process, or the epiphytic microbes can be isolated separately, by imprinting and lifting off pieces of plant roots, stem or leaves onto the surface of an agar medium and then isolating individual colonies as above. This approach is especially useful for bacteria, for example. Alternatively, the roots can be processed without washing off small quantities of soil attached to the roots, thus including microbes that colonize the plant rhizosphere. Otherwise, soil adhering to the roots can be removed, diluted and spread out onto agar of suitable selective and non-selective media to isolate individual colonies of rhizospheric bacteria.

Biologically pure cultures of Rahnella aquatilis and Enterobacter sacchari were deposited on Jul. 14, 2015 with the American Type Culture Collection (ATCC; an International Depositary Authority), Manassas, Va., USA, and assigned ATTC Patent Deposit Designation numbers PTA-122293 and PTA-122294, respectively. These deposits were made under the provisions of the Budapest Treaty on the International Recognition of the Deposit of Microorganisms for the Purpose of Patent Procedure and the Regulations (Budapest Treaty).

### Compositions

Compositions comprising bacteria or bacterial populations produced according to methods described herein and/or having characteristics as described herein can be in the form of a liquid, a foam, or a dry product. In some examples, a composition comprising bacterial populations can be in the form of a dry powder, a slurry of powder and water, or a flowable seed treatment.

The composition can be fabricated in bioreactors such as continuous stirred tank reactors, batch reactors, and on the farm. In some examples, compositions can be stored in a container, such as a jug or in mini bulk. In some examples, compositions can be stored within an object selected from the group consisting of a bottle, jar, ampule, package, vessel, bag, box, bin, envelope, carton, container, silo, shipping container, truck bed, and/or case.

Compositions can also be used to improve plant traits. In some examples, one or more compositions can be coated onto a seed. In some examples, one or more compositions can be coated onto a seedling. In some examples, one or more compositions can be coated onto a surface of a seed. In some examples, one or more compositions can be coated as a layer above a surface of a seed. In some examples, a composition that is coated onto a seed can be in liquid form, in dry product form, in foam form, in a form of a slurry of powder and water, or in a flowable seed treatment. In some examples, one or more compositions can be applied to a seed and/or seedling by spraying, immersing, coating, encapsulating, and/or dusting the seed and/or seedling with the one or more compositions. In some examples, multiple bacteria or bacterial populations can be coated onto a seed and/or a seedling of the plant. In some examples, at least two, at least three, at least four, at least five, at least six, at least seven, at least eight, at least nine, at least ten, or more than ten bacteria of a bacterial combination can be selected from one of the following genera: Acidovorax, Agrobacterium, Bacillus, Burkholderia, Chryseobacterium, Curtobacterium, Enterobacter, Escherichia, Methylobacterium, Paenibacillus, Pantoea, Pseudomonas, Ralstonia, Saccharibacillus, Sphingomonas, and Stenotrophomonas.

In some examples, at least two, at least three, at least four, at least five, at least six, at least seven, at least eight, at least nine, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, and Pleosporaceae.

In some examples, at least two, at least three, at least four, at least five, at least six, at least seven, at least eight, at least night, at least ten, or more than ten bacteria and bacterial populations of an endophytic combination are selected from one of the following families: Bacillaceae, Burkholderiaceae, Comamonadaceae, Enterobacteriaceae, Flavobacteriaceae, Methylobacteriaceae, Microbacteriaceae, Paenibacillileae, Pseudomonnaceae, Rhizobiaceae, Sphingomonadaceae, Xanthomonadaceae, Cladosporiaceae, Gnomoniaceae, Incertae sedis, Lasiosphaeriaceae, Netriaceae, Pleosporaceae.

Examples of compositions can include seed coatings for commercially important agricultural crops, for example, Sorghum, canola, tomato, strawberry, barley, rice, maize, and wheat. Examples of compositions can also include seed coatings for corn, soybean, canola, Sorghum, potato, rice, vegetables, cereals, and oilseeds. Seeds as provided herein can be genetically modified organisms (GMO), non-GMO, organic, or conventional. In some examples, compositions can be sprayed on the plant aerial parts, or applied to the roots by inserting into furrows in which the plant seeds are planted, watering to the soil, or dipping the roots in a suspension of the composition. In some examples, compositions can be dehydrated in a suitable manner that maintains cell viability and the ability to artificially inoculate and colonize host plants. The bacterial species can be present in compositions at a concentration of between 10⁸ to 10¹⁰ CFU/ml. In some examples, compositions can be supplemented with trace metal ions, such as molybdenum ions, iron ions, manganese ions, or combinations of these ions. The concentration of ions in examples of compositions as described herein can between about 0.1 mM and about 50 mM. Some examples of compositions can also be formulated with a carrier, such as beta-glucan, carboxylmethyl cellulose (CMC), bacterial extracellular polymeric substance (EPS), sugar, animal milk, or other suitable carriers. In some examples, peat or planting materials can be used as a carrier, or biopolymers in which a composition is entrapped in the biopolymer can be used as a carrier.

The compositions comprising the bacterial populations described herein can be coated onto the surface of a seed. As such, compositions comprising a seed coated with one or more bacteria described herein are also contemplated. The seed coating can be formed by mixing the bacterial population with a porous, chemically inert granular carrier. Alternatively, the compositions can be inserted directly into the furrows into which the seed is planted or sprayed onto the plant leaves or applied by dipping the roots into a suspension of the composition. An effective amount of the composition can be used to populate the sub-soil region adjacent to the roots of the plant with viable bacterial growth, or populate the leaves of the plant with viable bacterial growth. In general, an effective amount is an amount sufficient to result in plants with improved traits (e.g. a desired level of nitrogen fixation).

Bacterial compositions described herein can be formulated using an agriculturally acceptable carrier. The formulation useful for these embodiments can include at least one member selected from the group consisting of a tackifier, a microbial stabilizer, a fungicide, an antibacterial agent, a preservative, a stabilizer, a surfactant, an anti-complex agent, an herbicide, a nematicide, an insecticide, a plant growth regulator, a fertilizer, a rodenticide, a dessicant, a bactericide, a nutrient, or any combination thereof. In some examples, compositions can be shelf-stable. For example, any of the compositions described herein can include an agriculturally acceptable carrier (e.g., one or more of a fertilizer such as a non-naturally occurring fertilizer, an adhesion agent such as a non-naturally occurring adhesion agent, and a pesticide such as a non-naturally occurring pesticide). A non-naturally occurring adhesion agent can be, for example, a polymer, copolymer, or synthetic wax. For example, any of the coated seeds, seedlings, or plants described herein can contain such an agriculturally acceptable carrier in the seed coating. In any of the compositions or methods described herein, an agriculturally acceptable carrier can be or can include a non-naturally occurring compound (e.g., a non-naturally occurring fertilizer, a non-naturally occurring adhesion agent such as a polymer, copolymer, or synthetic wax, or a non-naturally occurring pesticide). Non-limiting examples of agriculturally acceptable carriers are described below. Additional examples of agriculturally acceptable carriers are known in the art.

In some cases, bacteria are mixed with an agriculturally acceptable carrier. The carrier can be a solid carrier or liquid carrier, and in various forms including microspheres, powders, emulsions and the like. The carrier can be any one or more of a number of carriers that confer a variety of properties, such as increased stability, wettability, or dispersability. Wetting agents such as natural or synthetic surfactants, which can be nonionic or ionic surfactants, or a combination thereof can be included in the composition. Water-in-oil emulsions can also be used to formulate a composition that includes the isolated bacteria (see, for example, U.S. Pat. No. 7,485,451). Suitable formulations that can be prepared include wettable powders, granules, gels, agar strips or pellets, thickeners, and the like, microencapsulated particles, and the like, liquids such as aqueous flowables, aqueous suspensions, water-in-oil emulsions, etc. The formulation can include grain or legume products, for example, ground grain or beans, broth or flour derived from grain or beans, starch, sugar, or oil.

In some embodiments, the agricultural carrier can be soil or a plant growth medium. Other agricultural carriers that can be used include water, fertilizers, plant-based oils, humectants, or combinations thereof. Alternatively, the agricultural carrier can be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations can include food sources for the bacteria, such as barley, rice, or other biological materials such as seed, plant parts, sugar cane bagasse, hulls or stalks from grain processing, ground plant material or wood from building site refuse, sawdust or small fibers from recycling of paper, fabric, or wood.

For example, a fertilizer can be used to help promote the growth or provide nutrients to a seed, seedling, or plant. Non-limiting examples of fertilizers include nitrogen, phosphorous, potassium, calcium, sulfur, magnesium, boron, chloride, manganese, iron, zinc, copper, molybdenum, and selenium (or a salt thereof). Additional examples of fertilizers include one or more amino acids, salts, carbohydrates, vitamins, glucose, NaCl, yeast extract, NH₄H₂PO₄, (NH₄)₂SO₄, glycerol, valine, L-leucine, lactic acid, propionic acid, succinic acid, malic acid, citric acid, KH tartrate, xylose, lyxose, and lecithin. In one embodiment, the formulation can include a tackifier or adherent (referred to as an adhesive agent) to help bind other active agents to a substance (e.g., a surface of a seed). Such agents are useful for combining bacteria with carriers that can contain other compounds (e.g., control agents that are not biologic), to yield a coating composition. Such compositions help create coatings around the plant or seed to maintain contact between the microbe and other agents with the plant or plant part. In one embodiment, adhesives are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

In some embodiments, the adhesives can be, e.g. a wax such as carnauba wax, beeswax, Chinese wax, shellac wax, spermaceti wax, candelilla wax, castor wax, ouricury wax, and rice bran wax, a polysaccharide (e.g., starch, dextrins, maltodextrins, alginate, and chitosans), a fat, oil, a protein (e.g., gelatin and zeins), gum arables, and shellacs. Adhesive agents can be non-naturally occurring compounds, e.g., polymers, copolymers, and waxes. For example, non-limiting examples of polymers that can be used as an adhesive agent include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, and polychloroprene.

In some examples, one or more of the adhesion agents, anti-fungal agents, growth regulation agents, and pesticides (e.g., insecticide) are non-naturally occurring compounds (e.g., in any combination). Additional examples of agriculturally acceptable carriers include dispersants (e.g., polyvinylpyrrolidone/vinyl acetate PVPIVA S-630), surfactants, binders, and filler agents.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant, which can include any compound or mixture of compounds that can be classified as a desiccant regardless of whether the compound or compounds are used in such concentrations that they in fact have a desiccating effect on a liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non reducing sugars and sugar alcohols (e.g., mannitol or sorbitol). The amount of desiccant introduced into the formulation can range from about 5% to about 50% by weight/volume, for example, between about 10% to about 40%, between about 15% to about 35%, or between about 20% to about 30%. In some cases, it is advantageous for the formulation to contain agents such as a fungicide, an antibacterial agent, an herbicide, a nematicide, an insecticide, a plant growth regulator, a rodenticide, bactericide, or a nutrient. In some examples, agents can include protectants that provide protection against seed surface-borne pathogens. In some examples, protectants can provide some level of control of soil-borne pathogens. In some examples, protectants can be effective predominantly on a seed surface.

In some examples, a fungicide can include a compound or agent, whether chemical or biological, that can inhibit the growth of a fungus or kill a fungus. In some examples, a fungicide can include compounds that can be fungistatic or fungicidal. In some examples, fungicide can be a protectant, or agents that are effective predominantly on the seed surface, providing protection against seed surface-borne pathogens and providing some level of control of soil-borne pathogens. Non-limiting examples of protectant fungicides include captan, maneb, thiram, or fludioxonil.

In some examples, a fungicide can be a systemic fungicide, which can be absorbed into the emerging seedling and inhibit or kill the fungus inside host plant tissues. Systemic fungicides used for seed treatment include, but are not limited to the following: azoxystrobin, carboxin, mefenoxam, metalaxyl, thiabendazole, trifloxystrobin, and various triazole fungicides, including difenoconazole, ipconazole, tebuconazole, and triticonazole. Mefenoxam and metalaxyl are primarily used to target the water mold fungi Pythium and Phytophthora. Some fungicides are preferred over others, depending on the plant species, either because of subtle differences in sensitivity of the pathogenic fungal species, or because of the differences in the fungicide distribution or sensitivity of the plants. In some examples, fungicide can be a biological control agent, such as a bacterium or fungus. Such organisms can be parasitic to the pathogenic fungi, or secrete toxins or other substances which can kill or otherwise prevent the growth of fungi. Any type of fungicide, particularly ones that are commonly used on plants, can be used as a control agent in a seed composition.

In some examples, the seed coating composition comprises a control agent which has antibacterial properties. In one embodiment, the control agent with antibacterial properties is selected from the compounds described herein elsewhere. In another embodiment, the compound is Streptomycin, oxytetracycline, oxolinic acid, or gentamicin. Other examples of antibacterial compounds which can be used as part of a seed coating composition include those based on dichlorophene and benzylalcohol hemi formal (Proxel® from ICI or Acticide® RS from Thor Chemie and Kathon® MK 25 from Rohm & Haas) and isothiazolinone derivatives such as alkylisothiazolinones and benzisothiazolinones (Acticide® MBS from Thor Chemie).

In some examples, a growth regulator is selected from the group consisting of: Abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione phosphorotrithioate, 2,3,5-tri-iodobenzoic acid, trinexapac-ethyl and uniconazole. Additional non-limiting examples of growth regulators include brassinosteroids, cytokinines (e.g., kinetin and zeatin), auxins (e.g., indolylacetic acid and indolylacetyl aspartate), flavonoids and isoflavanoids (e.g., formononetin and diosmetin), phytoaixins (e.g., glyceolline), and phytoalexin-inducing oligosaccharides (e.g., pectin, chitin, chitosan, polygalacuronic acid, and oligogalacturonic acid), and gibellerins. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

Some examples of nematode-antagonistic biocontrol agents include ARF18; 30 Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; and Rhizobacteria. Particularly preferred nematode-antagonistic biocontrol agents include ARF18, Arthrobotrys oligospora, Arthrobotrys dactyloides, Chaetomium globosum, Cylindrocarpon heteronema, Exophilia jeanselmei, Exophilia pisciphila, Fusarium aspergilus, Fusarium solani, Gliocladium catenulatum, Gliocladium roseum, Gliocladium vixens, Hirsutella rhossiliensis, Hirsutella minnesotensis, Lecanicillium lecanii, Monacrosporium drechsleri, Monacrosporium gephyropagum, Myrotehcium verrucaria, Neocosmospora vasinfecta, Paecilomyces lilacinus, Pochonia chlamydosporia, Stagonospora heteroderae, Stagonospora phaseoli, vesicular-arbuscular mycorrhizal fungi, Burkholderia cepacia, Pasteuria penetrans, Pasteuria thornei, Pasteuria nishizawae, Pasteuria ramosa, Pastrueia usage, Brevibacillus laterosporus strain G4, Pseudomonas fluorescens and Rhizobacteria.

Some examples of nutrients can be selected from the group consisting of a nitrogen fertilizer including, but not limited to Urea, Ammonium nitrate, Ammonium sulfate, Non-pressure nitrogen solutions, Aqua ammonia, Anhydrous ammonia, Ammonium thiosulfate, Sulfur-coated urea, Urea-formaldehydes, IBDU, Polymer-coated urea, Calcium nitrate, Ureaform, and Methylene urea, phosphorous fertilizers such as Diammonium phosphate, Monoammonium phosphate, Ammonium polyphosphate, Concentrated superphosphate and Triple superphosphate, and potassium fertilizers such as Potassium chloride, Potassium sulfate, Potassium-magnesium sulfate, Potassium nitrate. Such compositions can exist as free salts or ions within the seed coat composition. Alternatively, nutrients/fertilizers can be complexed or chelated to provide sustained release over time.

Some examples of rodenticides can include selected from the group of substances consisting of 2-isovalerylindan-1,3-dione, 4-(quinoxalin-2-ylamino) benzenesulfonamide, alpha-chlorohydrin, aluminum phosphide, antu, arsenous oxide, barium carbonate, bisthiosemi, brodifacoum, bromadiolone, bromethalin, calcium cyanide, chloralose, chlorophacinone, cholecalciferol, coumachlor, coumafuryl, coumatetralyl, crimidine, difenacoum, difethialone, diphacinone, ergocalciferol, flocoumafen, fluoroacetamide, flupropadine, flupropadine hydrochloride, hydrogen cyanide, iodomethane, lindane, magnesium phosphide, methyl bromide, norbormide, phosacetim, phosphine, phosphorus, pindone, potassium arsenite, pyrinuron, scilliroside, sodium arsenite, sodium cyanide, sodium fluoroacetate, strychnine, thallium sulfate, warfarin and zinc phosphide.

In the liquid form, for example, solutions or suspensions, bacterial populations can be mixed or suspended in water or in aqueous solutions. Suitable liquid diluents or carriers include water, aqueous solutions, petroleum distillates, or other liquid carriers.

Solid compositions can be prepared by dispersing the bacterial populations in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The solid carriers used upon formulation include, for example, mineral carriers such as kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran can be used. The liquid carriers include vegetable oils such as soybean oil and cottonseed oil, glycerol, ethylene glycol, polyethylene glycol, propylene glycol, polypropylene glycol, etc.

### Application of Bacterial Populations on Crops

The composition of the bacteria or bacterial population described herein can be applied in furrow, in talc, or as seed treatment. In some embodiments, the composition of the bacteria or bacterial population described herein can be applied indirectly to a plant. In some embodiments, the composition of the bacteria or bacterial population described herein can be applied as a side dressing. Applying the bacteria or bacterial population indirectly and/or as a side dressing can include applying the bacteria or bacterial population to a shallow furrow or band along the side of a plant or in a circle around a plant. The composition or bacterial population can be applied to a seed package in bulk, mini bulk, in a bag, or in talc.

The composition of the bacteria or bacterial population described herein can be applied after a plant seed is planted but prior to harvest. For example, the composition or bacterial population can be applied between one and eight months after germination, including between two and eight months, one and three months, and three and six months after germination.

The planter can plant the treated seed and grow the crop according to conventional ways, twin row, or ways that do not require tilling. The seeds can be distributed using a control hopper or an individual hopper. Seeds can also be distributed using pressurized air or manually. Seed placement can be performed using variable rate technologies. Additionally, application of the bacteria or bacterial population described herein can be applied using variable rate technologies. In some examples, the bacteria can be applied to seeds of corn, soybean, canola, Sorghum, potato, rice, vegetables, cereals, pseudocereals, and oilseeds. Examples of cereals can include barley, fonio, oats, palmer's grass, rye, pearl millet, Sorghum, spelt, teff, triticale, and wheat. Examples of pseudocereals can include breadnut, buckwheat, cattail, chia, flax, grain amaranth, hanza, quinoa, and sesame. In some examples, seeds can be genetically modified organisms (GMO), non-GMO, organic or conventional.

Additives such as micro-fertilizer, PGR, herbicide, insecticide, and fungicide can be used additionally to treat the crops. Examples of additives include crop protectants such as insecticides, nematicides, fungicide, enhancement agents such as colorants, polymers, pelleting, priming, and disinfectants, and other agents such as inoculant, PGR, softener, and micronutrients. PGRs can be natural or synthetic plant hormones that affect root growth, flowering, or stem elongation. PGRs can include auxins, gibberellins, cytokinins, ethylene, and abscisic acid (ABA).

The composition can be applied in a furrow in combination with liquid fertilizer. In some examples, the liquid fertilizer can be held in tanks. NPK fertilizers contain macronutrients of sodium, phosphorous, and potassium.

The composition can improve plant traits, such as promoting plant growth, maintaining high chlorophyll content in leaves, increasing fruit or seed numbers, and increasing fruit or seed unit weight. Methods of the present disclosure can be employed to introduce or improve one or more of a variety of desirable traits. Examples of traits that can introduced or improved include: root biomass, root length, height, shoot length, leaf number, water use efficiency, overall biomass, yield, fruit size, grain size, photosynthesis rate, tolerance to drought, heat tolerance, salt tolerance, tolerance to low nitrogen stress, nitrogen use efficiency, resistance to nematode stress, resistance to a fungal pathogen, resistance to a bacterial pathogen, resistance to a viral pathogen, level of a metabolite, modulation in level of a metabolite, proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under identical conditions. In some examples, the desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the introduced and/or improved traits) grown under similar conditions.

An agronomic trait to a host plant can include, but is not limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation

In some cases, plants are inoculated with bacteria or bacterial populations that are isolated from the same species of plant as the plant element of the inoculated plant. For example, a bacteria or bacterial population that is normally found in one variety of Zea mays (corn) is associated with a plant element of a plant of another variety of Zea mays that in its natural state lacks said bacteria and bacterial populations. In one embodiment, the bacteria and bacterial populations is derived from a plant of a related species of plant as the plant element of the inoculated plant. For example, an bacteria and bacterial populations that is normally found in Zea diploperennis Iltis et al., (diploperennial teosinte) is applied to a Zea mays (corn), or vice versa. In some cases, plants are inoculated with bacteria and bacterial populations that are heterologous to the plant element of the inoculated plant. In one embodiment, the bacteria and bacterial populations is derived from a plant of another species. For example, an bacteria and bacterial populations that is normally found in dicots is applied to a monocot plant (e.g., inoculating corn with a soybean-derived bacteria and bacterial populations), or vice versa. In other cases, the bacteria and bacterial populations to be inoculated onto a plant is derived from a related species of the plant that is being inoculated. In one embodiment, the bacteria and bacterial populations is derived from a related taxon, for example, from a related species. The plant of another species can be an agricultural plant. In another embodiment, the bacteria and bacterial populations is part of a designed composition inoculated into any host plant element.

In some examples, the bacteria or bacterial population is exogenous wherein the bacteria and bacterial population is isolated from a different plant than the inoculated plant. For example, in one embodiment, the bacteria or bacterial population can be isolated from a different plant of the same species as the inoculated plant. In some cases, the bacteria or bacterial population can be isolated from a species related to the inoculated plant.

In some examples, the bacteria and bacterial populations described herein are capable of moving from one tissue type to another. For example, the present invention's detection and isolation of bacteria and bacterial populations within the mature tissues of plants after coating on the exterior of a seed demonstrates their ability to move from seed exterior into the vegetative tissues of a maturing plant. Therefore, in one embodiment, the population of bacteria and bacterial populations is capable of moving from the seed exterior into the vegetative tissues of a plant. In one embodiment, the bacteria and bacterial populations that is coated onto the seed of a plant is capable, upon germination of the seed into a vegetative state, of localizing to a different tissue of the plant. For example, bacteria and bacterial populations can be capable of localizing to any one of the tissues in the plant, including: the root, adventitious root, seminal 5 root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In one embodiment, the bacteria and bacterial populations is capable of localizing to the root and/or the root hair of the plant. In another embodiment, the bacteria and bacterial populations is capable of localizing to the photosynthetic tissues, for example, leaves and shoots of the plant. In other cases, the bacteria and bacterial populations is localized to the vascular tissues of the plant, for example, in the xylem and phloem. In still another embodiment, the bacteria and bacterial populations is capable of localizing to the reproductive tissues (flower, pollen, pistil, ovaries, stamen, fruit) of the plant. In another embodiment, the bacteria and bacterial populations is capable of localizing to the root, shoots, leaves and reproductive tissues of the plant. In still another embodiment, the bacteria and bacterial populations colonizes a fruit or seed tissue of the plant. In still another embodiment, the bacteria and bacterial populations is able to colonize the plant such that it is present in the surface of the plant (i.e., its presence is detectably present on the plant exterior, or the episphere of the plant). In still other embodiments, the bacteria and bacterial populations is capable of localizing to substantially all, or all, tissues of the plant. In some embodiments, the bacteria and bacterial populations is not localized to the root of a plant. In other cases, the bacteria and bacterial populations is not localized to the photosynthetic tissues of the plant.

The effectiveness of the compositions can also be assessed by measuring the relative maturity of the crop or the crop heating unit (CHU). For example, the bacterial population can be applied to corn, and corn growth can be assessed according to the relative maturity of the corn kernel or the time at which the corn kernel is at maximum weight. The crop heating unit (CHU) can also be used to predict the maturation of the corn crop. The CHU determines the amount of heat accumulation by measuring the daily maximum temperatures on crop growth.

In examples, bacterial can localize to any one of the tissues in the plant, including: the root, adventitious root, seminal root, root hair, shoot, leaf, flower, bud tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In another embodiment, the bacteria or bacterial population is capable of localizing to the photosynthetic tissues, for example, leaves and shoots of the plant. In other cases, the bacteria and bacterial populations is localized to the vascular tissues of the plant, for example, in the xylem and phloem. In another embodiment, the bacteria or bacterial population is capable of localizing to reproductive tissues (flower, pollen, pistil, ovaries, stamen, or fruit) of the plant. In another embodiment, the bacteria and bacterial populations is capable of localizing to the root, shoots, leaves and reproductive tissues of the plant. In another embodiment, the bacteria or bacterial population colonizes a fruit or seed tissue of the plant. In still another embodiment, the bacteria or bacterial population is able to colonize the plant such that it is present in the surface of the plant. In another embodiment, the bacteria or bacterial population is capable of localizing to substantially all, or all, tissues of the plant. In some embodiments, the bacteria or bacterial population is not localized to the root of a plant. In other cases, the bacteria and bacterial populations is not localized to the photosynthetic tissues of the plant.

The effectiveness of the bacterial compositions applied to crops can be assessed by measuring various features of crop growth including, but not limited to, planting rate, seeding vigor, root strength, drought tolerance, plant height, dry down, and test weight.

### Plant Species

The methods and bacteria described herein are suitable for any of a variety of plants, such as plants in the genera Hordeum, Oryza, Zea, and Triticeae. Other non-limiting examples of suitable plants include mosses, lichens, and algae. In some cases, the plants have economic, social and/or environmental value, such as food crops, fiber crops, oil crops, plants in the forestry or pulp and paper industries, feedstock for biofuel production and/or ornamental plants. In some examples, plants can be used to produce economically valuable products such as a grain, a flour, a starch, a syrup, a meal, an oil, a film, a packaging, a nutraceutical product, a pulp, an animal feed, a fish fodder, a bulk material for industrial chemicals, a cereal product, a processed human-food product, a sugar, an alcohol, and/or a protein. Non-limiting examples of crop plants include maize, rice, wheat, barley, Sorghum, millet, oats, rye triticale, buckwheat, sweet corn, sugar cane, onions, tomatoes, strawberries, and asparagus.

In some examples, plants that can be obtained or improved using the methods and composition disclosed herein can include plants that are important or interesting for agriculture, horticulture, biomass for the production of biofuel molecules and other chemicals, and/or forestry. Some examples of these plants can include pineapple, banana, coconut, lily, grasspeas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (Sorghum, sudan), Miscanthus giganteus (Miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (Eucalyptus), Triticosecale spp. (Triticum-25 wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea 5 spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (Poinsettia), Nicotiana tabacum (tobacco), Lupinus albus (lupin), Uniola paniculata (oats), Hordeum vulgare (barley), and Lolium spp. (rye).

In some examples, a monocotyledonous plant can be used. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some examples, the monocotyledonous plant can be selected from the group consisting of a maize, rice, wheat, barley, and sugarcane.

In some examples, a dicotyledonous plant can be used, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some examples, the dicotyledonous plant can be selected from the group consisting of cotton, soybean, pepper, and tomato.

In some cases, the plant to be improved is not readily amenable to experimental conditions. For example, a crop plant can take too long to grow enough to practically assess an improved trait serially over multiple iterations. Accordingly, a first plant from which bacteria are initially isolated, and/or the plurality of plants to which genetically manipulated bacteria are applied can be a model plant, such as a plant more amenable to evaluation under desired conditions. Non-limiting examples of model plants include Setaria, Brachypodium, and Arabidopsis. Ability of bacteria isolated according to a method of the disclosure using a model plant can then be applied to a plant of another type (e.g. a crop plant) to confirm conferral of the improved trait.

Traits that may be improved by the methods disclosed herein include any observable characteristic of the plant, including, for example, growth rate, height, weight, color, taste, smell, and changes in the production of one or more compounds by the plant (including for example, metabolites, proteins, drugs, carbohydrates, oils, and any other compounds). Selecting plants based on genotypic information is also envisaged (for example, including the pattern of plant gene expression in response to the bacteria, or identifying the presence of genetic markers, such as those associated with increased nitrogen fixation). Plants may also be selected based on the absence, suppression or inhibition of a certain feature or trait (such as an undesirable feature or trait) as opposed to the presence of a certain feature or trait (such as a desirable feature or trait).

### EXAMPLES

The examples provided herein describe methods of bacterial isolation, bacterial and plant analysis, and plant trait improvement. The examples are for illustrative purposes only and are not to be construed as limiting in any way.

### Example 1: Isolation of Microbes from Plant Tissue

Topsoil was obtained from various agricultural areas in central California. Twenty soils with diverse texture characteristics were collected, including heavy clay, peaty clay loam, silty clay, and sandy loam. Seeds of various field corn, sweet corn, heritage corn and tomato were planted into each soil, as shown in

### Table 1.

TABLE 1
Crop Type and Varieties planted into soil
with diverse characteristics

| Crop | Field | | Heritage | |
|---|---|---|---|---|
| Type | Corn | Sweet Corn | Corn | Tomato |
| Varie- | Mo17 | Ferry-Morse | Victory Seeds | Ferry-Morse |
| ties | | ‘Golden Cross | ‘Moseby | Roma VF |
| | | Bantam T-51’ | Prolific’ | |
| | B73 | Ferry-Morse | Victory Seeds | Stover Roma |
| | | ‘Silver Queen | ‘Reid's Yellow | |
| | | Hybrid’ | Dent’ | |
| | DKC | Ferry-Morse | Victory Seeds | Totally |
| | 66-40 | ‘Sugar Dots’ | ‘Hickory King’ | Tomatoes |
| | | | | ‘Micro Tom |
| | | | | Hybrid’ |
| | DKC | | | Heinz 1015 |
| | 67-07 | | | |
| | DKC | | | Heinz 2401 |
| | 70-01 | | | |
| | | | | Heinz 3402 |
| | | | | Heinz 5508 |
| | | | | Heinz 5608 |
| | | | | Heinz 8504 |

Plants were uprooted after 2-4 weeks of growth and excess soil on root surfaces was removed with deionized water. Following soil removal, plants were surface sterilized with bleach and rinsed vigorously in sterile water. A cleaned, 1 cm section of root was excised from the plant and placed in a phosphate buffered saline solution containing 3 mm steel beads. A slurry was generated by vigorous shaking of the solution with a Qiagen TissueLyser II.

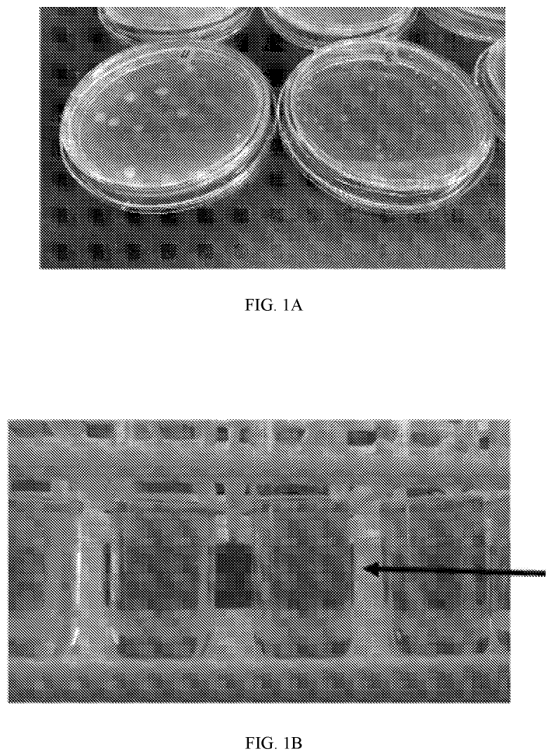

The root and saline slurry was diluted and inoculated onto various types of growth media to isolate rhizospheric, endophytic, epiphytic, and other plant-associated microbes. R2A and Nfb agar media were used to obtain single colonies, and semisolid Nfb media slants were used to obtain populations of nitrogen fixing bacteria. After 2-4 weeks incubation in semi-solid Nfb media slants, microbial populations were collected and streaked to obtain single colonies on R2A agar, as shown in FIGS. 1A-B. Single colonies were resuspended in a mixture of R2A and glycerol, subjected to PCR analysis, and frozen at −80° C. for later analysis. Approximately 1,000 single colonies were obtained and designated “isolated microbes.”

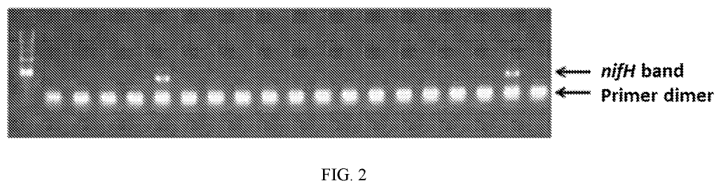

Isolates were then subjected to a colony PCR screen to detect the presence of the nifH gene in order to identify diazotrophs. The previously-described primer set Ueda 19F/388R, which has been shown to detect over 90% of diazotrophs in screens, was used to probe the presence of the nif cluster in each isolate (Ueda et al. 1995; J. Bacteriol. 177: 1414-1417). Single colonies of purified isolates were picked, resuspended in PBS, and used as a template for colony PCR, as shown in FIG. 2. Colonies of isolates that gave positive PCR bands were re-streaked, and the colony PCR and re-streaking process was repeated twice to prevent false positive identification of diazotrophs. Purified isolates were then designated “candidate microbes.”

### Example 2: Characterization of Isolated Microbes

### Sequencing, Analysis and Phylogenetic Characterization

Sequencing of 16S rDNA with the 515f-806r primer set was used to generate preliminary phylogenetic identities for isolated and candidate microbes (see e.g. Vernon et al.; BMC Microbiol. 2002 Dec. 23; 2:39.). The microbes comprise diverse genera including: Enterobacter, Burkholderia, Klebsiella, Bradyrhizobium, Rahnella, Xanthomonas, Raoultella, Pantoea, Pseudomonas, Brevundimonas, Agrobacterium, and Paenibacillus, as shown in Table 2.

TABLE 2
Diversity of microbes isolated from tomato plants
as determined by deep 16S rDNA sequencing.

| Genus | Isolates |
|---|---|
| Achromobacter | 7 |
| Agrobacterium | 117 |
| Agromyces | 1 |
| Aticyclobacillus | 1 |
| Asticcacaulis | 6 |
| Bacillus | 131 |
| Bradyrhizobium | 2 |
| Brevibacillus | 2 |
| Burkholderia | 2 |
| Caulobacter | 17 |
| Chryseobocterium | 42 |
| Comamonas | 1 |
| Dyadobacter | 2 |
| Flavobacterium | 46 |
| Halomonas | 3 |
| Leptothrix | 3 |
| Lysobacter | 2 |
| Neisseria | 13 |
| Paenibacilius | 1 |
| Paenisporosarcina | 3 |
| Pantoea | 14 |
| Pedobacter | 16 |
| Pimelobacter | 2 |
| Pseudomonas | 212 |
| Rhizobium | 4 |
| Rhodoferax | 1 |
| Sphingobacterium | 13 |
| Sphingobium | 23 |
| Sphingomonas | 3 |
| Sphingopyxis | 1 |
| Stenotrophomonas | 59 |
| Streptococcus | 3 |
| Variovarax | 37 |
| Xylonimicrobium | 1 |
| unidentified | 75 |

Subsequently, the genomes of 39 candidate microbes were sequenced using Illumina Miseq platform. Genomic DNA from pure cultures was extracted using the QIAmp DNA mini kit (QIAGEN), and total DNA libraries for sequencing were prepared through a third party vendor (SeqMatic, Hayward). Genome assembly was then carried out via the A5 pipeline (Tritt et al. 2012; PLoS One 7(9):e42304). Genes were identified and annotated, and those related to regulation and expression of nitrogen fixation were noted as targets for mutagenesis.

Two microbial base strains, designated CI137 and CI1021, were identified and are used for further testing of the effect of various genetic mutations on nitrogen uptake. CI137 represents a WT K. variicola and CI1021 represents a WT Kosakonia pseudosacchari.

### Example 3: Mutagenesis of Candidate Microbes

Lambda-Red Mutagenesis with Cas9 Selection

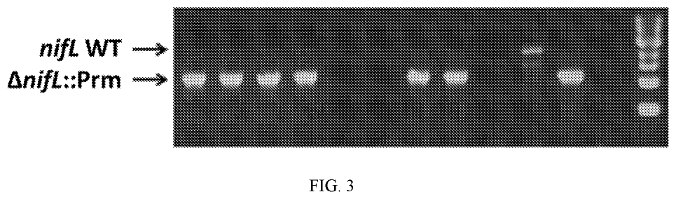

Mutants of candidate microbes were generated via lambda-red mutagenesis with selection by CRISPR-Cas. Knockout cassettes contained an endogenous promoter identified through transcriptional profiling and ˜250 bp homology regions flanking the deletion target. Candidate microbes were transformed with plasmids encoding the Lambda-red recombination system (exo, beta, gam genes) under control of an arabinose inducible promoter and Cas9 under control of an IPTG inducible promoter. The Red recombination and Cas9 systems were induced in resulting transformants, and strains were prepared for electroporation. Knockout cassettes and a plasmid-encoded selection gRNA were subsequently transformed into the competent cells. In an exemplary reaction for the deletion of nifL and substitution of the promoter regulating nif operon transcription in candidate strain CI006, after plating on antibiotics selective for both the Cas9 plasmid and the gRNA plasmid, 7 of the 10 colonies screened showed the intended knockout mutation, as shown in FIG. 3.

This approach was similarly applied to modify the CI137 and CI1021 base strains to create the mutant strains identified in Table 3.

### Example 4: In Vitro Phenotyping of Candidate Molecules

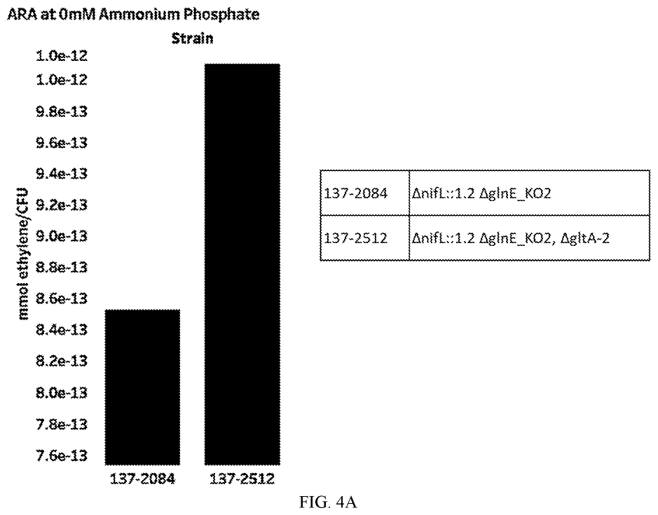

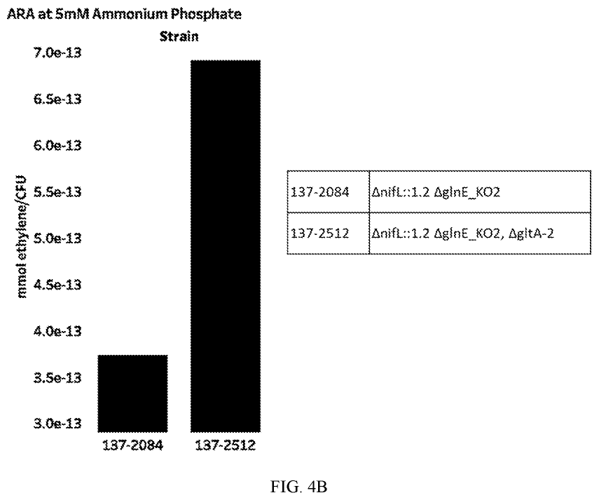

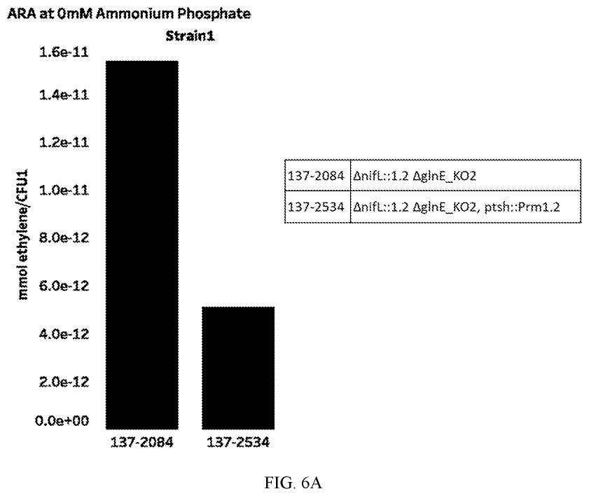

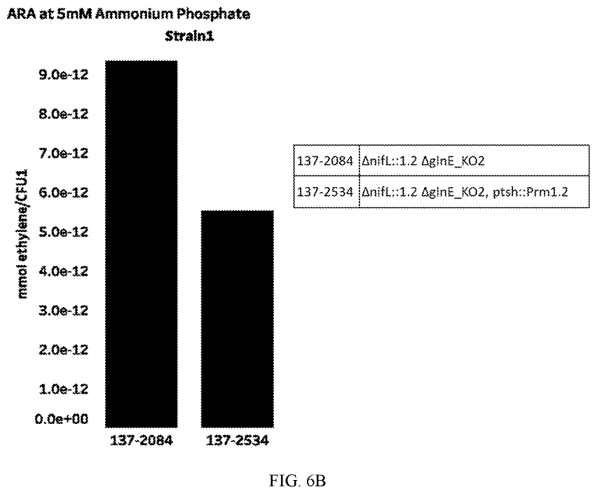

The impact of exogenous nitrogen on nitrogenase biosynthesis and activity in various mutants was assessed. The Acetylene Reduction Assay (ARA) (Temme et. al. 2012; 109(18): 7085-7090) was used to measure nitrogenase activity in pure culture conditions. Strains were grown in air-tight test tubes, and reduction of acetylene to ethylene was quantified with an Agilent 6890 gas chromatograph. ARA activities of candidate microbes and counterpart candidate mutants grown in nitrogen fixation media supplemented with 0 or 5 mM ammonium phosphate are shown in FIGS. 4A-B and FIGS. 6A-B. As shown in FIGS. 4A-B, strains containing a deletion of the gltA gene demonstrated increased ARA activity relative to a control strain. As shown in FIGS. 6A-B, substitution of the promoter regulating ptsH expression resulted in decreased ARA activity relative to a control strain.

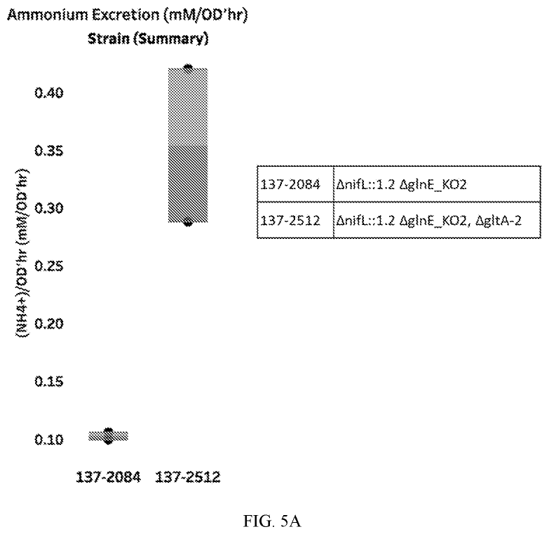

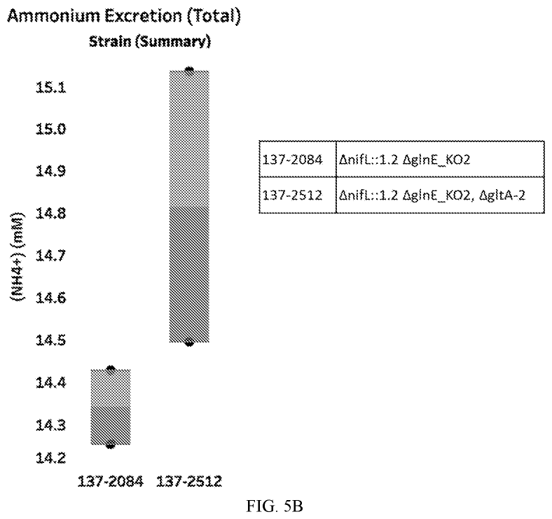

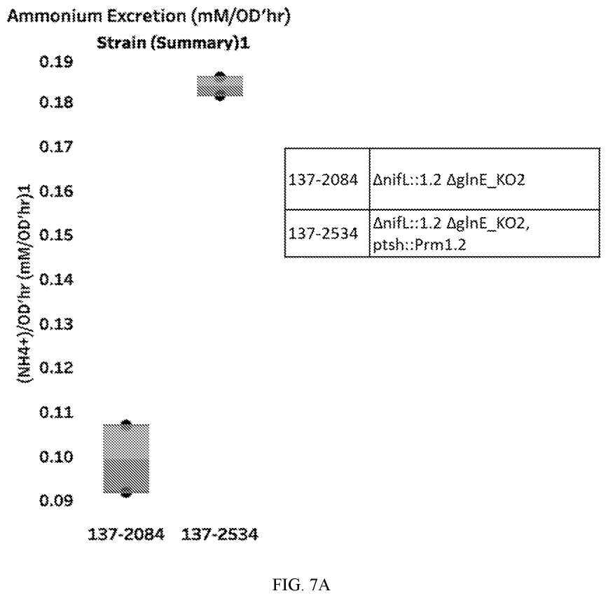

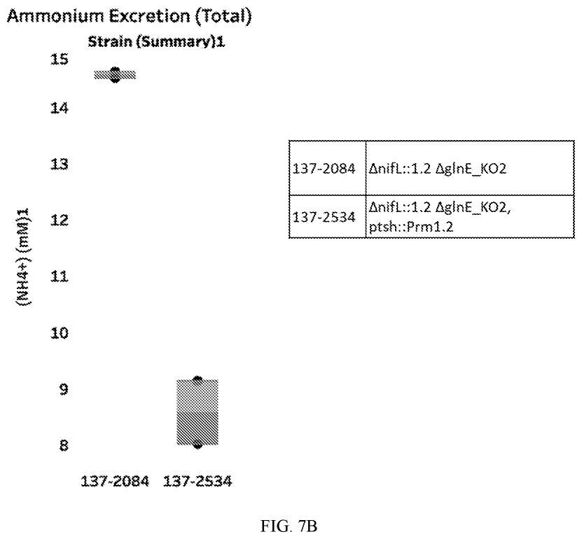

The strains from FIGS. 4A-B and 6A-B were also subjected to an ammonium excretion (AMM) assay, in which ammonia excretion over time was measured in nitrogen fixing conditions by culturing cells in nitrogen-free media, pelleting the cells, and measuring free ammonium in the cell-free broth; higher ammonium excretion levels indicated higher nitrogen fixation and excretion. As shown in FIGS. 5A-B the deletion of gltA resulted in an increased rate (FIG. 5B) and total (FIG. 5A) ammonium excretion relative to a control. As shown in FIG. 7B, changing the ptsH promoter resulted in an increased ammonium excretion rate relative to a control strain.

### Example 5: Biofilm Formation

Biofilm formation can influence the amount of nitrogen being provided to an associated plant. An increase in biofilm formation on the root of a plant can increase the amount of nitrogen provided to the plant.

Some genes can regulate biofilm formation. For example, smZ can be a negative regulator of biofilm regulation. Mutation of smZ such that smZ has reduced or eliminated function can increase biofilm formation in bacteria. Mutagenesis can be performed to mutate smZ in strains with increased nitrogen excretion and fixation activity to create an smZ mutant library of bacteria.

Some proteins can promote biofilm formation. For example, large adhesion proteins including lapA can promote biofilm formation. One or more promoters regulating the expression of lapA may be unregulated, such that more lapA is produced, as detected by western blot. Additional genes which may be modified to alter biofilm formation include pga, sdiA, fimA1-A4, wzxE, and bolA.

Modified strains may be grown in lysogeny buffer (LB) and inoculated into soil with a seedling. The seedling, soil, and bacterial strain can be in a pot, in a field, indoors, or outdoors. The seedling can be planted in spring, summer, fall, or winter. The air can have about 0%, 10%, 20%, 30%, 40%, 50%, 60%, 70%, 80%, 90%, or 100% humidity. It may be raining, foggy, cloudy, or sunny. The temperature can be about 4° C., 10° C., 15° C., 20° C., 25° C., 30° C., 35° C., 40° C., or 45° C. The seedling can be planted at dawn, morning, midday, afternoon, dusk, evening, or night. The seedling can be left to grow for about 1, 2, 3, 4, 5, 6, 7, 8, 9, 10, 20, or 30 days. After the time has elapsed, the plant can be dug up and the amount of biofilm on the roots can be measured.

### Example 6: Oxygen Sensitivity

Oxygen sensitivity of nitrogenase enzymes can affect nitrogen fixation, nitrogen excretion, or both. Decreasing the oxygen sensitivity of a nitrogenase enzyme can increase the nitrogen fixing activity, increase the nitrogen excreting activity, or both.

To evaluate this hypothesis, the sodA, sodB, and sodC genes were overexpressed by substituting the promoter regulating gene expression.

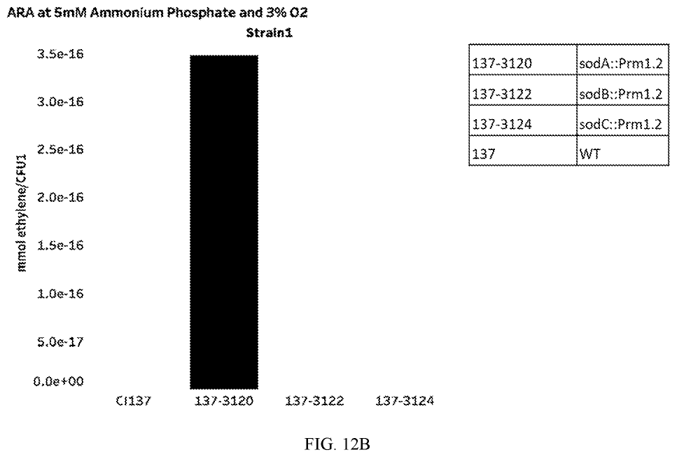

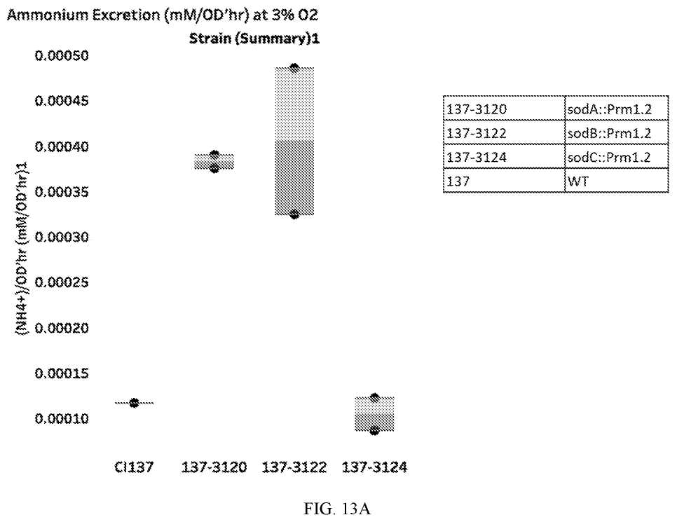

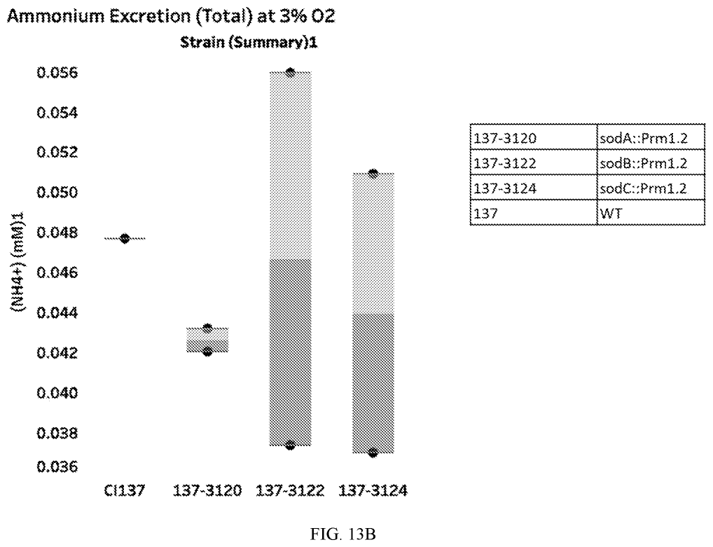

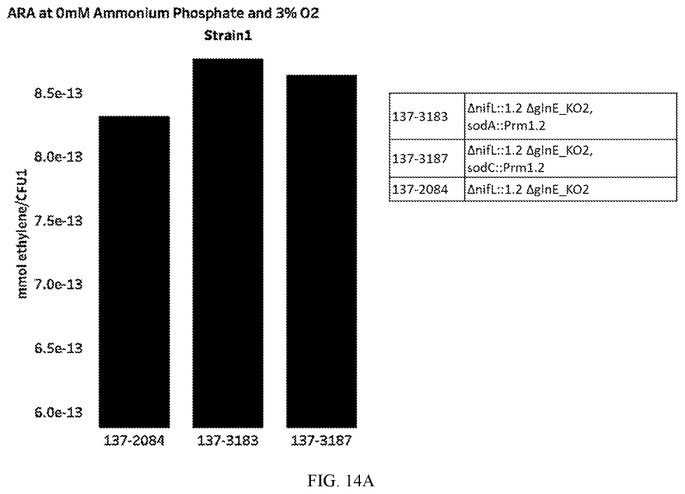

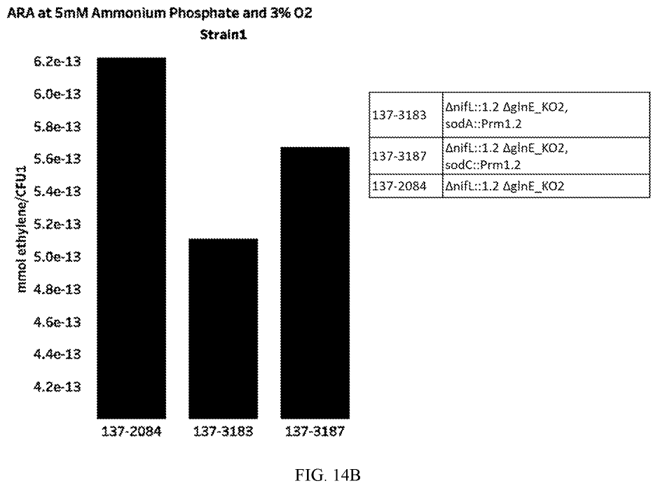

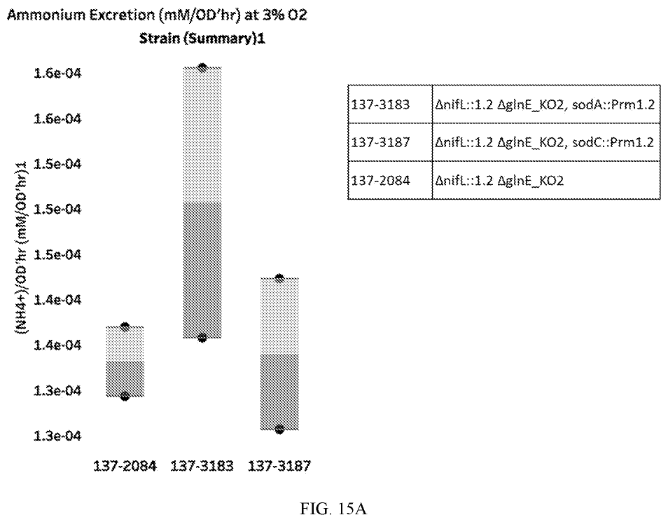

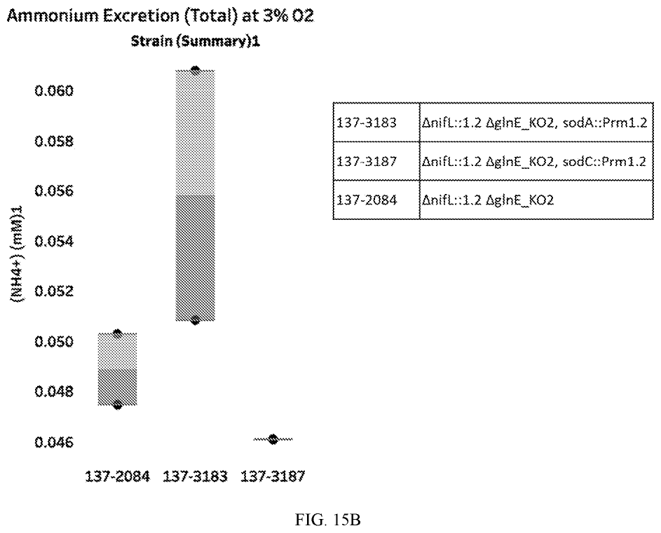

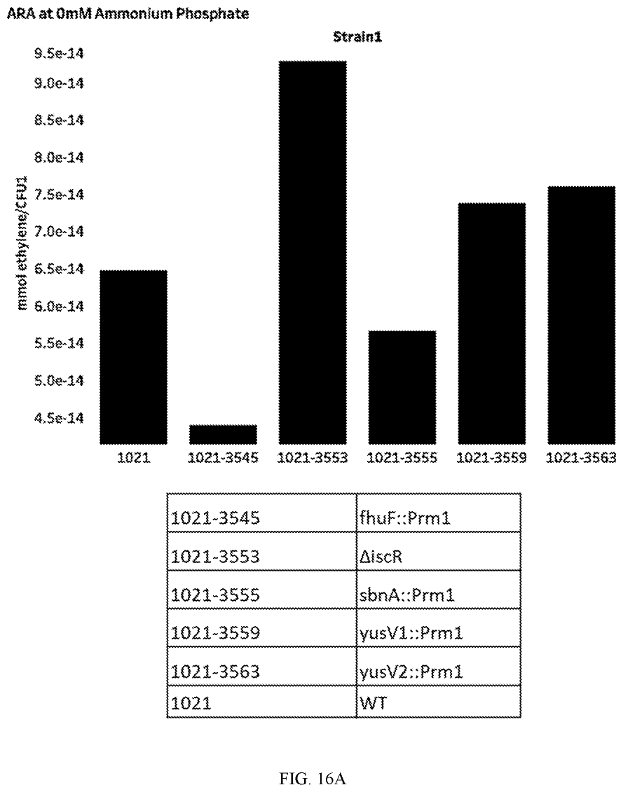

The overexpression strains were subjected to an oxygen sensitivity assay in which AMM and ARA assays can be performed under conditions with varying oxygen concentrations. The results are shown in FIGS. 12A-B, 13A-B, 14A-B, 15A-B, 20A-B,21A-B, 22A-B, and 23A-B. To further test the impact of oxygen tolerance on nitrogen fixing activity and nitrogen excreting activity, strains with modifications in the FNR, arcA, and arcB genes will be created and subjected to the ARA and AMM assays with varying oxygen conditions.

### Example 7: Increasing Iron Transport May Increase Nitrogen Fixation

The iron uptake pathway can refer to metabolic pathway in bacteria which enables the uptake of iron into the cell. Iron can act as a cofactor to promote nitrogen fixation. Increasing the iron uptake of cells may increase nitrogen fixation, nitrogen excretion, or both.

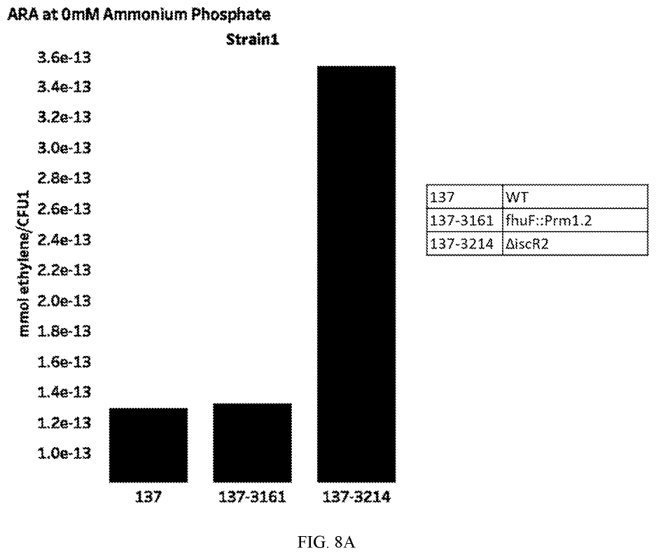

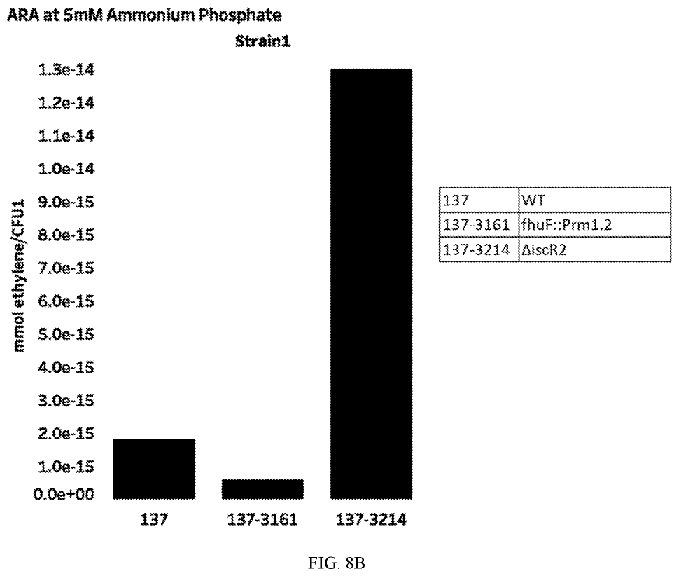

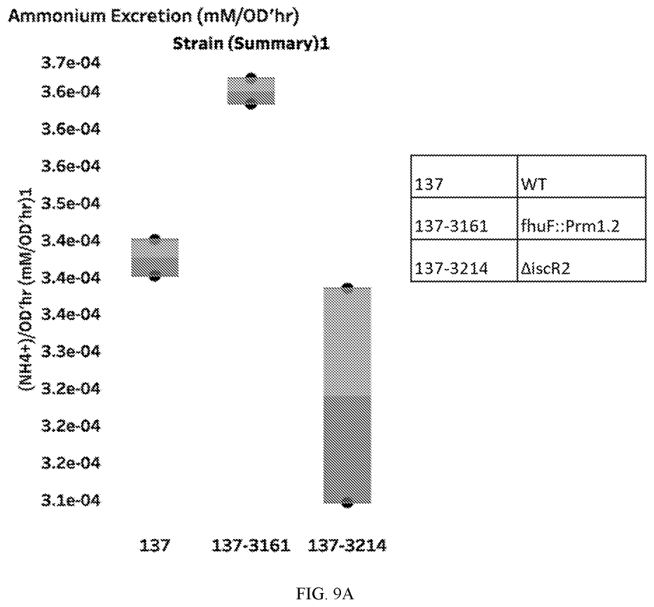

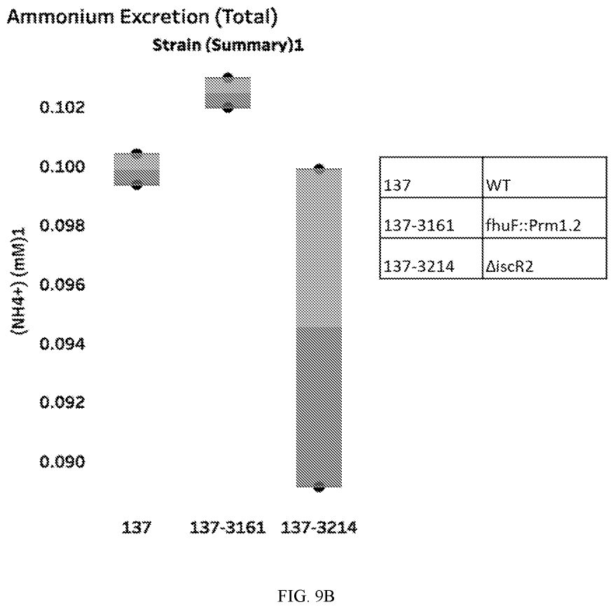

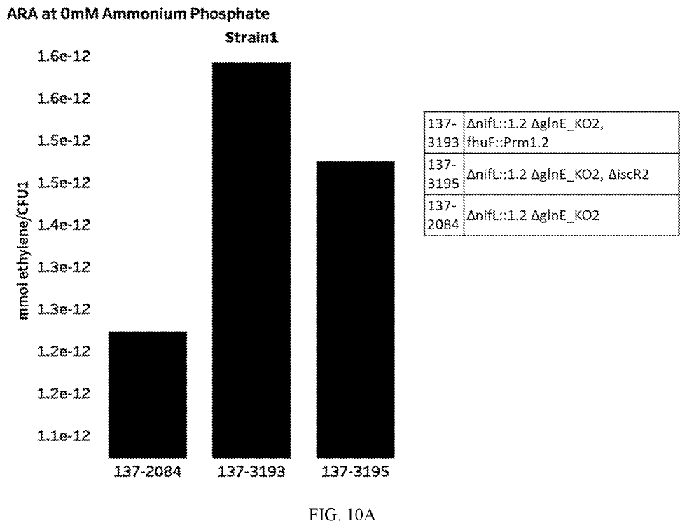

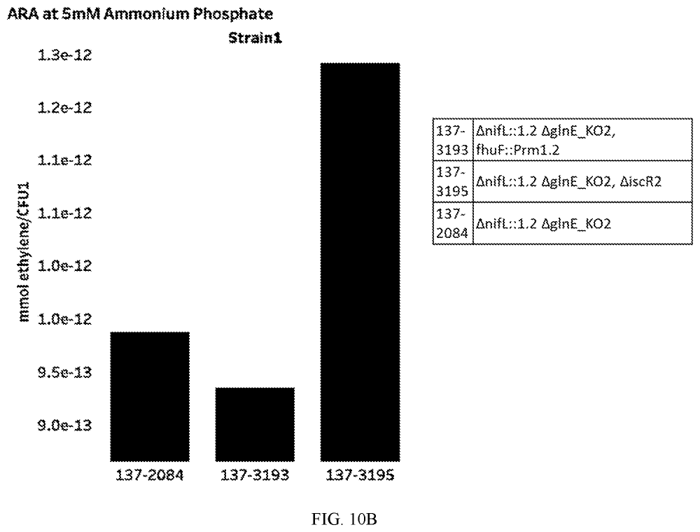

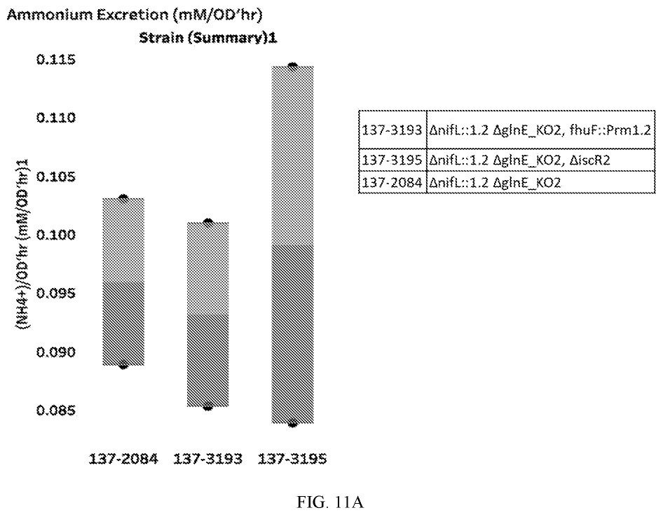

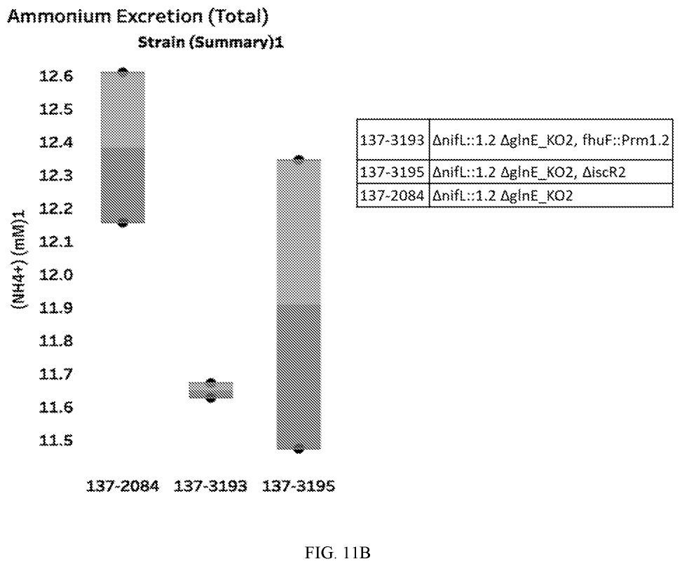

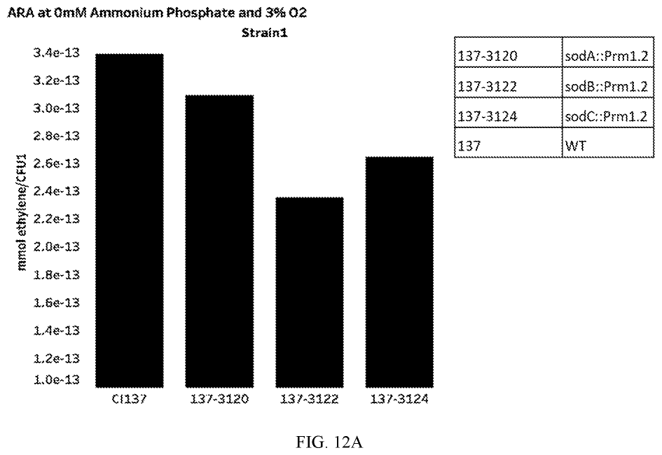

To evaluate this hypothesis, genetic modifications were created in fhuF and iscR, positive and negative regulators of iron uptake, respectively. Specifically, strains containing deletions of iscR or a promoter substitution of the fhuF promoter were created and assayed for ARA and AMM activity. The results are shown in FIGS. 8A-B, 9A-B, 10A-B, 11A-B, 16A-16B, 17A-B, 18A-B, and 19A-B. Deletion of iscR resulted in increased ARA activity (see, FIGS. 8A-B, 10A-B, 16A-B, and 18A-B).

A library of variants may also be constructed such that promoter activity for one or more genes in the iron uptake pathway is increased. Western blotting can be used to determine which strains have increased expression of one or more iron uptake genes, and these strains can be variants of interest.

To screen for increased iron uptake activity, each mutant or variant of interest as well as the unmutated parent strain for each strain can be inoculated into and grown in three wells of a 96 well plate in LB medium. Once the cells reach OD=0.8, one of the wells of each mutant can be harvested, such that the cells are pelleted, excess LB is washed away, and cells are resuspended, lysed, and prepared for spectroscopy in a 96 well plate. Iron content can be measured using spectroscopy to establish the baseline iron content in the samples. The remaining samples can be spiked with iron as FeSO₄ in LB or LB alone, such that the amount added is 10% or less of the total volume in each well. 0.1 mM of acetic acid (final concentration) may be included to aid in the solubility of the iron. The final concentration of iron in each well in the assay can be one of 10 pM, 100 pM, 1 nM, 10 nM, 100 nM, 1 μM, 10 μM, or 100 μM, and more than one concentration can be tested. The assay can be conducted over one or more of 1 second, 10 seconds, 30 seconds, 1 minute, 2 minutes, 3 minutes, 4 minutes, 5 minutes, 10 minutes, 20 minutes, 30 minutes, and 60 minutes.

First, for each concentration, the amount of iron transported into each cell as a function of time can be plotted to determine an optimal duration for the assay. The optimal duration can be a time interval wherein steady state has not been reached, and the reaction rate still linear. Then, for the optimal duration, the amount of iron transported into each cell as a function of concentration can be plotted, and a Michaelis-Menten analysis can be performed. Mutant strains or variants of interest which show a significant increase (p<0.05) in iron transport over the unmutated and unmodified parent strain as determined by Km comparison can be selected for further assays.

Next, each strain selected for further assays can be subjected to an AMM assay as well as an ARA assay as described herein to measure nitrogen excretion and fixation, respectively. One or more of these mutations or variants may increase the AMM, ARA, or both of one or more of these bacterial strains.

### Example 8: Increasing Siderophore Biosynthesis Genes May Increase Nitrogen Fixation

yhf siderophores, or iron chelating molecules may influence the uptake of iron in bacteria. Increasing the production of yhf siderophores can increase iron uptake in bacteria. Modifications in siderophore genes, including yhfA, yusV, sbnA, yfiZ, fiu, and fur, may increase the production of yhf siderophores, which may in turn increase iron uptake in cells. These modifications may lead to increased nitrogen fixation or excretion.

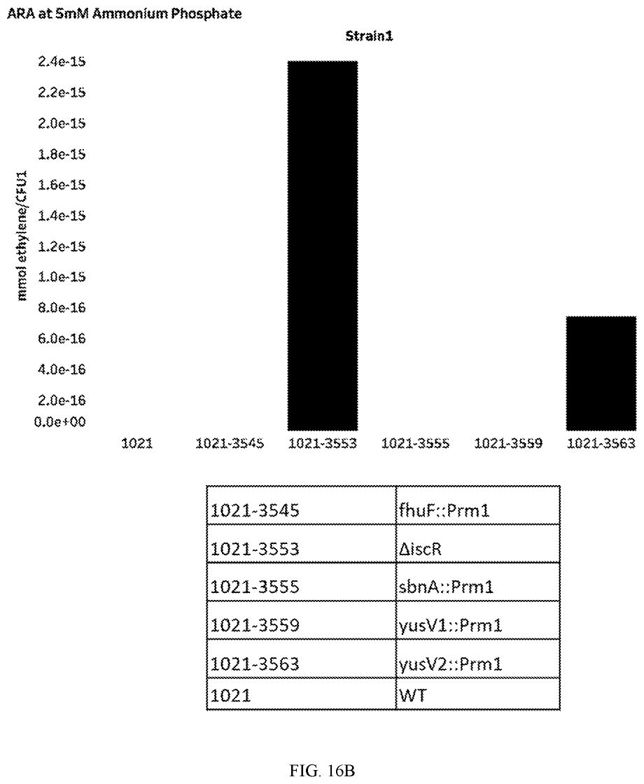

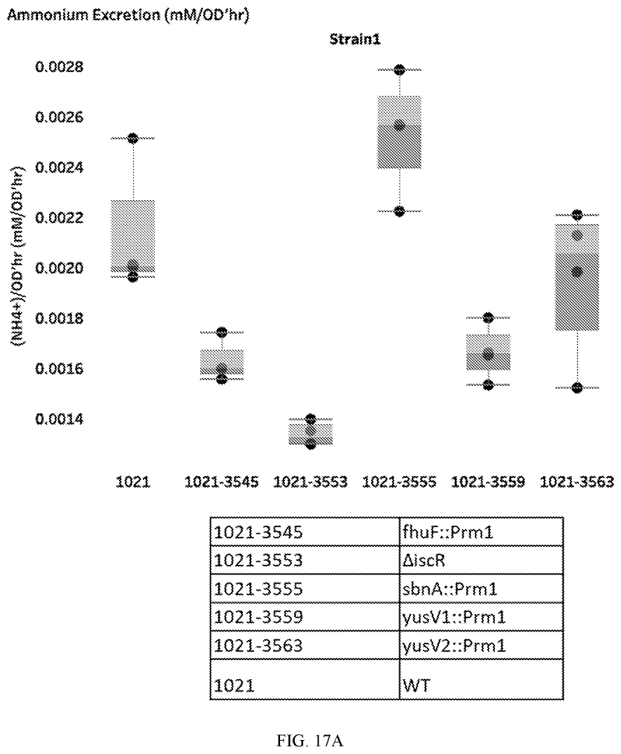

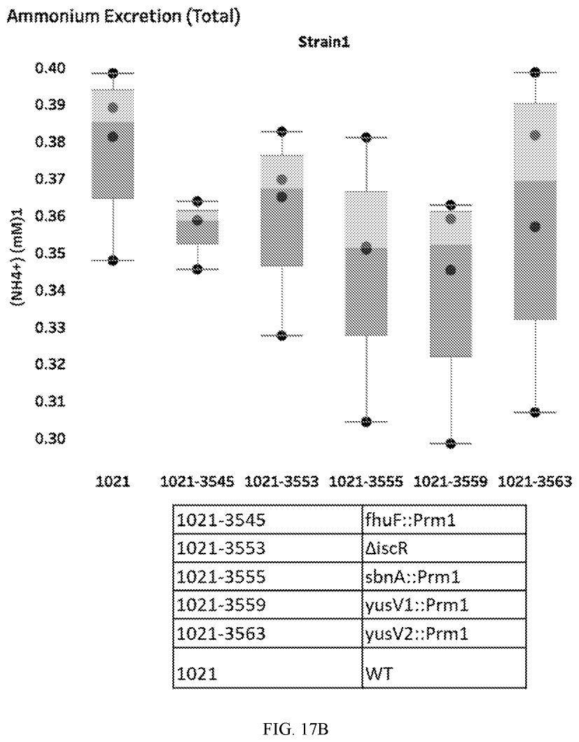

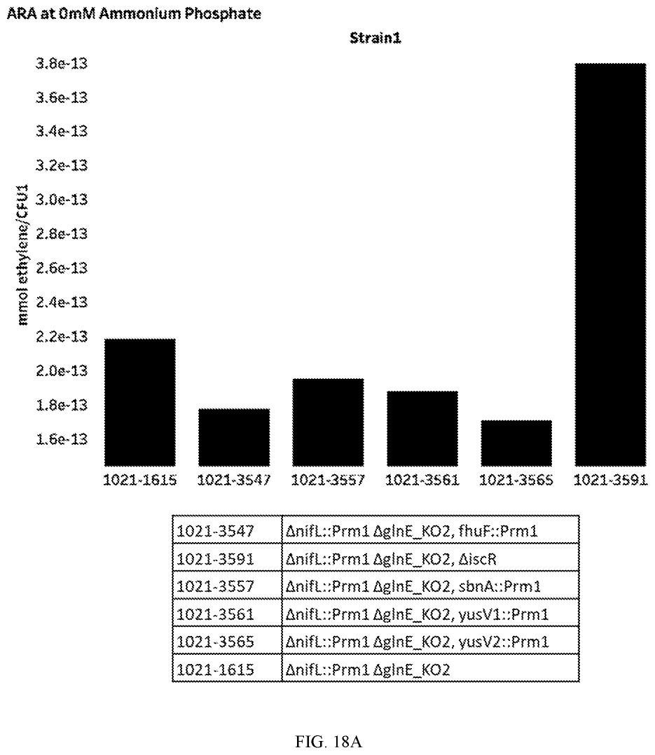

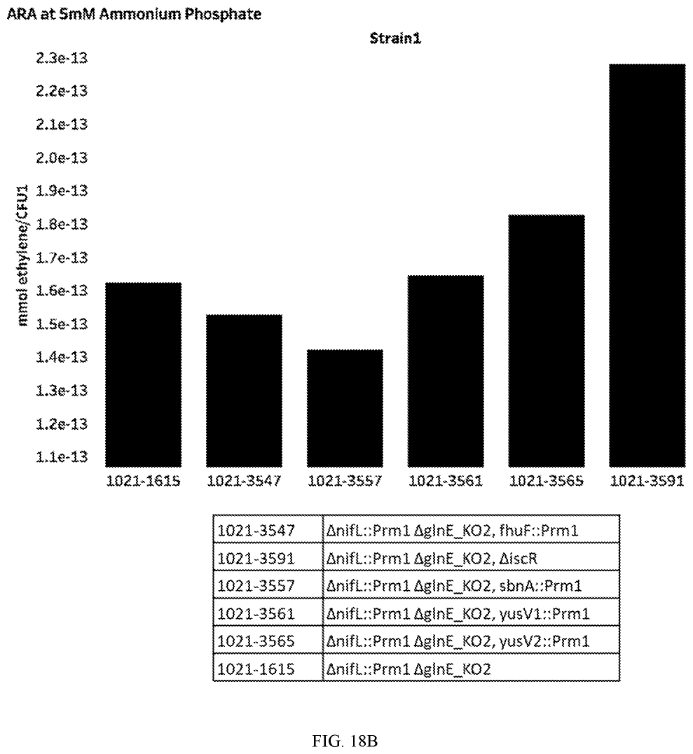

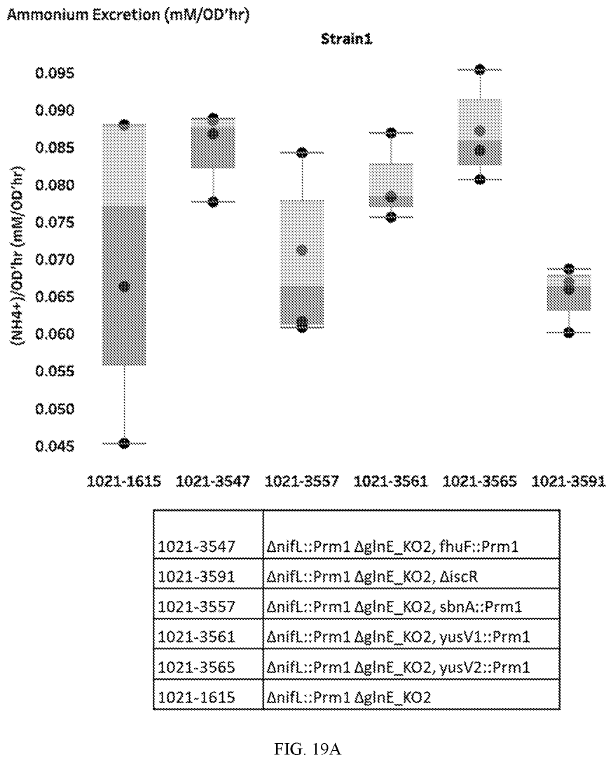

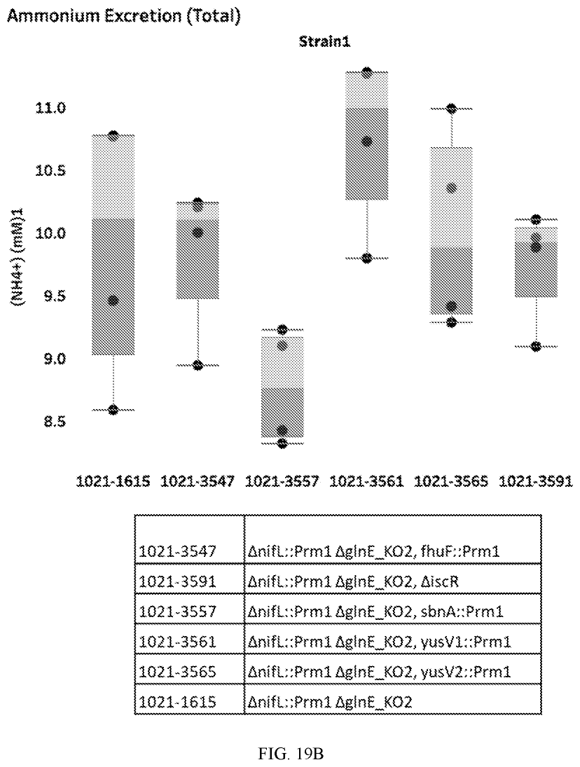

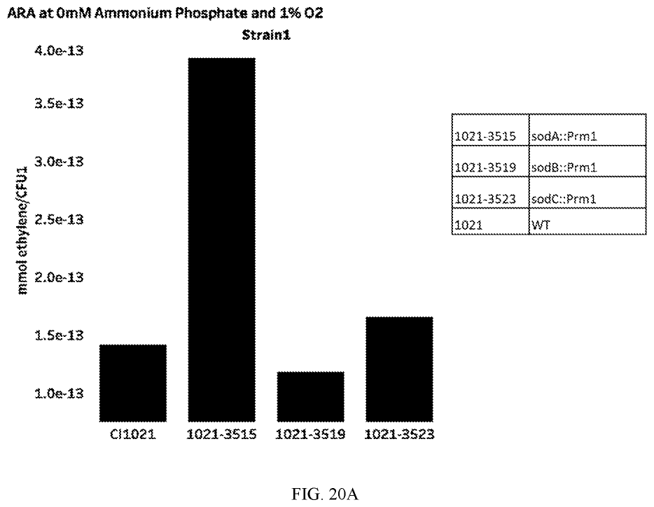

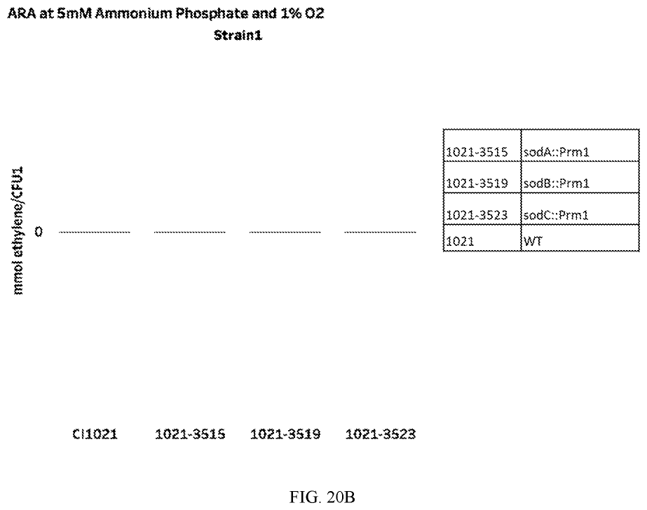

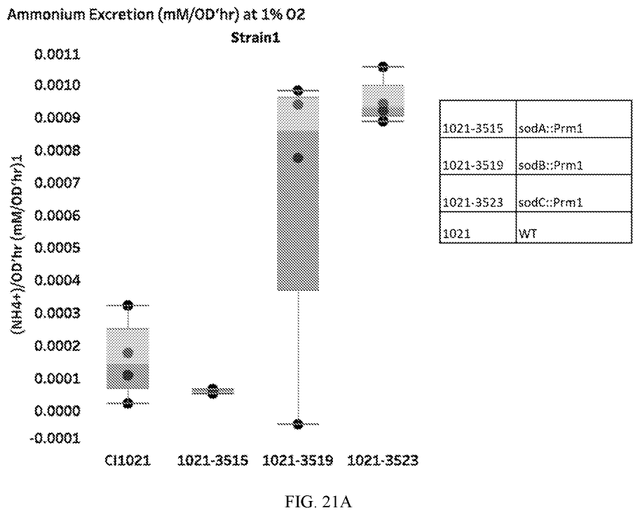

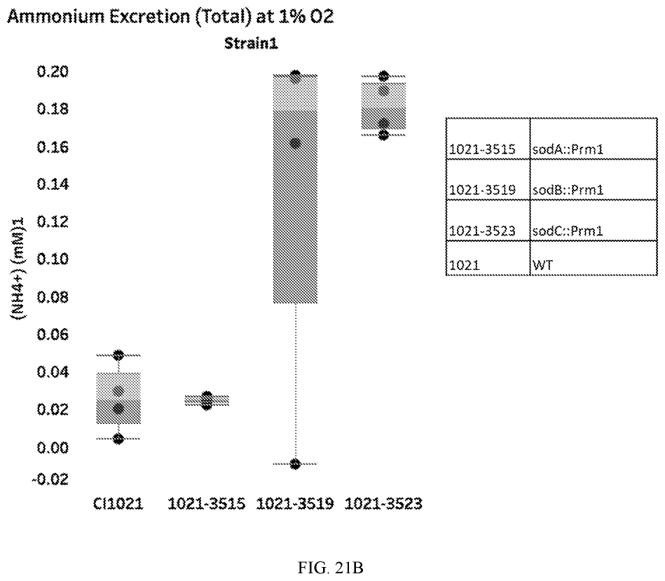

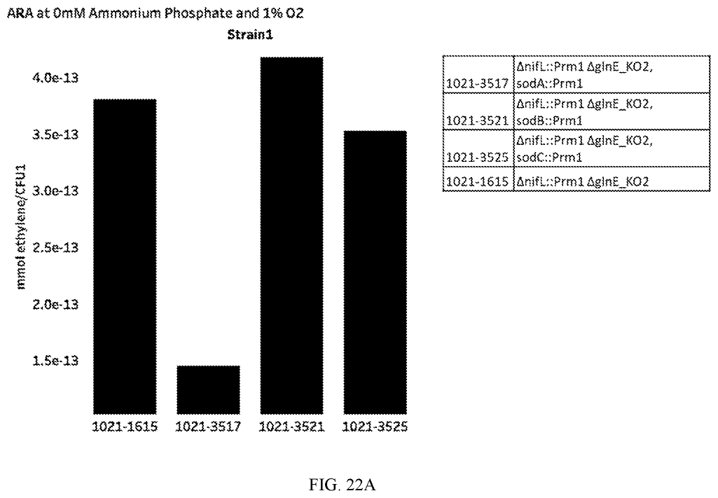

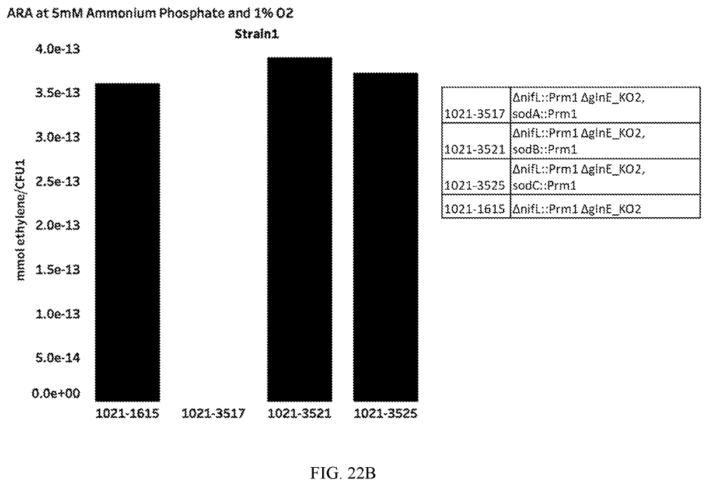

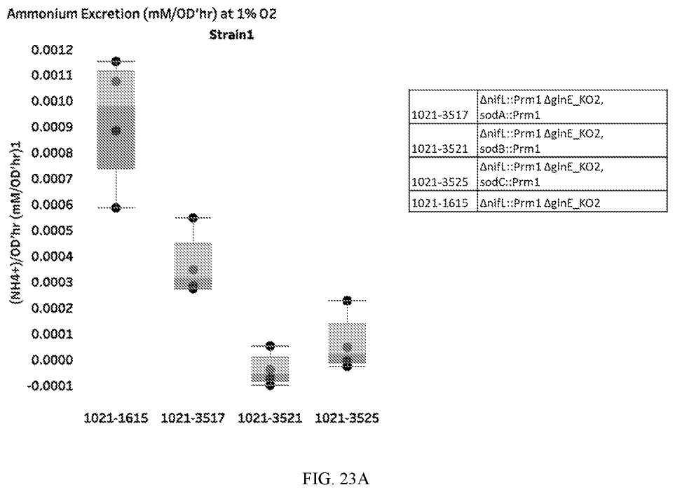

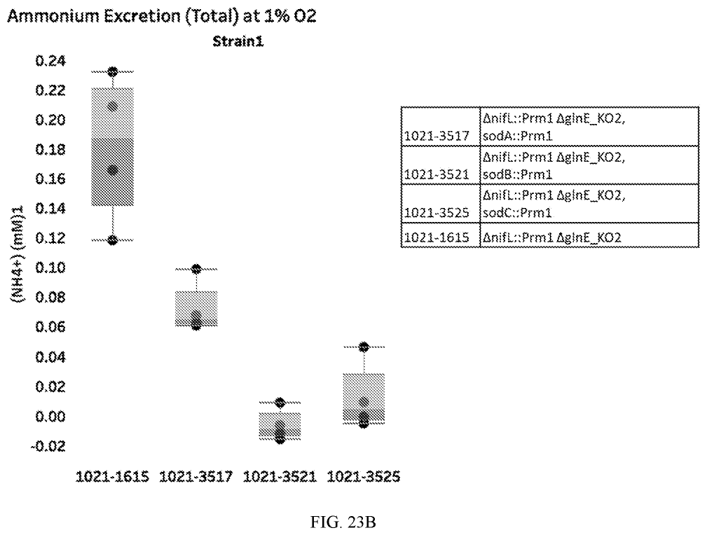

To evaluate whether increased expression of siderophore genes alters nitrogen fixation or excretion, sbnA, yusV1, and yusV2 were overexpressed by substituting the promoter regulating expression of the genes. The modified strains were then subjected to ARA and AMM assays. The results are shown in FIGS. 16A-B, 17A-B, 18A-B, and 19A-B.

Genetic modifications can be performed on these genes in bacterial cells such that the modifications result in an increased siderophore production. To identify such modifications, mutagenesis may be performed in and around the active sites of the genes that code for yhfA, yusV, sbnA, yfiZ, fiu, or fur. A library of mutants may be constructed for each gene. These libraries can be then screened for increased siderophore production. These libraries can be created from wild type cells, or from cells which have already been modified to display increased nitrogen fixation, increased nitrogen excretion, or both. To screen for increased siderophore production, each mutant can be grown to OD=0.8 and then spiked with an excess but not toxic amount of siderophore precursors and incubated for 30 minutes or 60 minutes. The amount of siderophores produced after the allotted time period can be normalized, and strains which display increased siderophore production may be screened for increased iron transport.

To screen for increased iron uptake activity, each mutant or variant of interest as well as the unmutated parent strain for each strain can be inoculated into and grown in three wells of a 96 well plate in LB medium. Once the cells reach OD=0.8, one of the wells of each mutant can be harvested, such that the cells are pelleted, excess LB is washed away, and cells are resuspended, lysed, and prepared for spectroscopy in a 96 well plate. Iron content can be measured using spectroscopy to establish the baseline iron content in the samples. The remaining samples can be spiked with iron as FeSO4 in LB or LB alone, such that the amount added is 10% or less of the total volume in each well. 0.1 mM of acetic acid (final concentration) may be included to aid in the solubility of the iron. The final concentration of iron in each well in the assay can be one of 10 pM, 100 pM, 1 nM, 10 nM, 100 nM, 1 μM, 10 μM, or 100 μM, and more than one concentration can be tested. The assay can be conducted over one or more of 1 second, 10 seconds, 30 seconds, 1 minute, 2 minutes, 3 minutes, 4 minutes, 5 minutes, 10 minutes, 20 minutes, 30 minutes, and 60 minutes.

First, for each concentration, the amount of iron transported into each cell as a function of time can be plotted to determine an optimal duration for the assay. The optimal duration can be a time interval wherein steady state has not been reached, and the reaction rate still linear. Then, for the optimal duration, the amount of iron transported into each cell as a function of concentration can be plotted, and a Michaelis-Menten analysis can be performed. Mutant strains or variants of interest which show a significant increase (p<0.05) in iron transport over the unmutated and unmodified parent strain as determined by Km comparison can be selected for further assays.

Next, each strain selected for further assays can be subjected to an AMM assay as well as an ARA assay as described herein to measure nitrogen excretion and fixation, respectively. One or more of these mutations or variants may increase the AMM, ARA, or both of one or more of these bacterial strains.

### Example 9: Increasing Desiccation Tolerance

Improving desiccation tolerance may increase colonization of a strain used in a seed coating, or other dry application to a plant or field. A genetically engineered microbe will be created comprising an arabinose promoter operably linked to an rpoE gene. Strains containing similar modifications to the rpoS, treA, treB, phoP, phoQ, and rpoN genes will also be generated. The genetically engineered microbes, and the non-engineered parental control, will be cultured in media supplemented with arabinose for 48 hours before being desiccated. After desiccation a portion of each of the engineered and non-engineered strains will be revived in media which does not contain arabinose, and the number of microbes in the media after 24 hours will be assayed. The media containing the engineered strain will contain more microbes than the media containing the non-engineered parental strain.

Desiccated microbes will be applied to corn seeds and planted in soil, lacking arabinose, in a greenhouse under conditions suitable for the germination of the corn seeds. After 4 weeks the seedlings will be harvested and the number of colony forming units of both the engineered and non-engineered microbes on the roots of the plants will be assessed. The plants exposed to the engineered microbes will be associated with more microbes than the plants exposed to the non-engineered parental strain.

### Example 10: NAC Gene Mutants

To evaluate the impact of mutations in the NAC gene on nitrogen fixation and excretion, NAC gene knockout and overexpression mutants were generated by deleting the gene or substituting the promoter regulating gene expression.

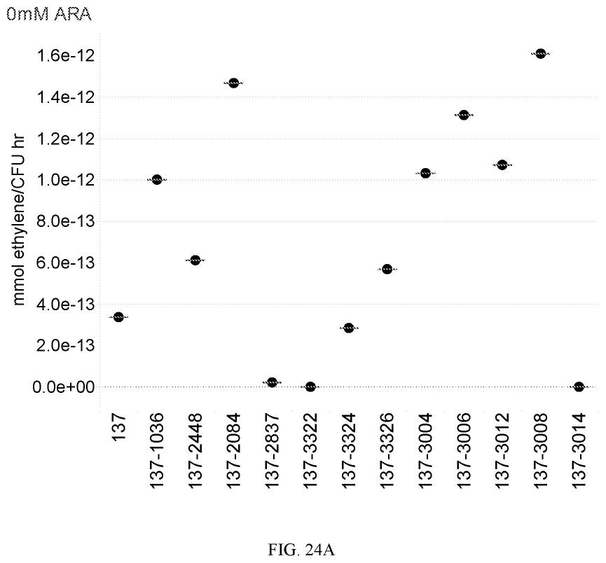

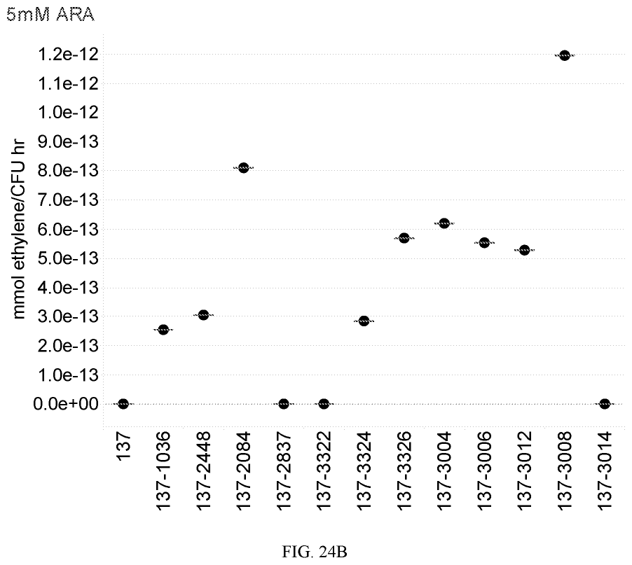

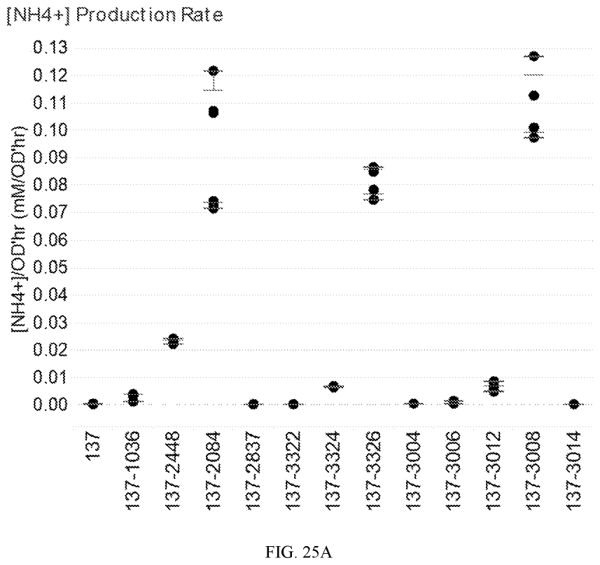

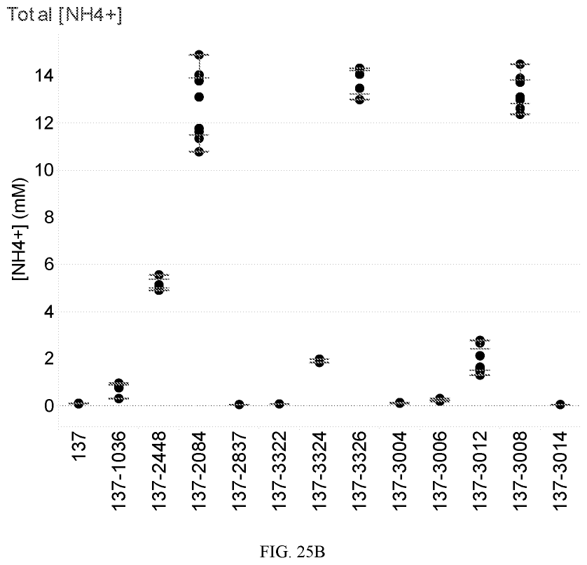

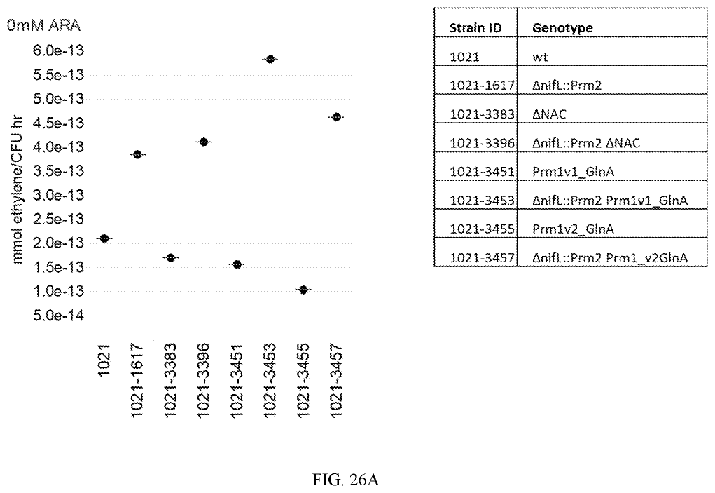

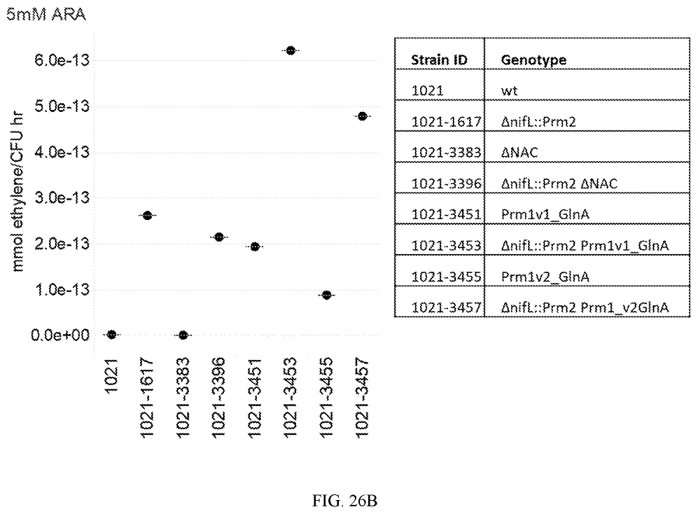

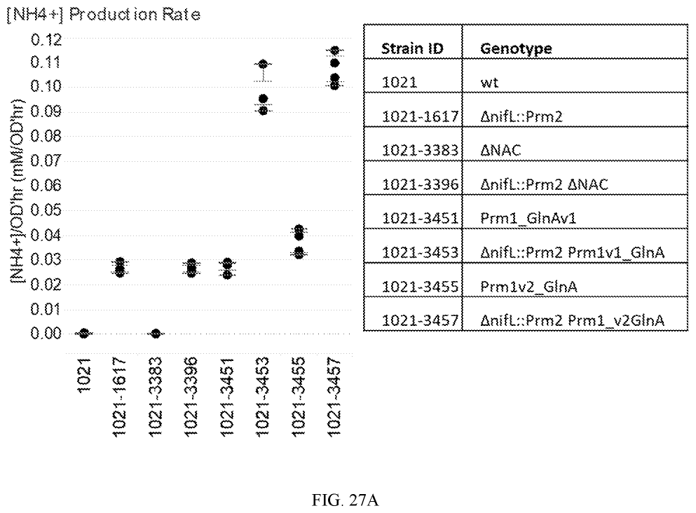

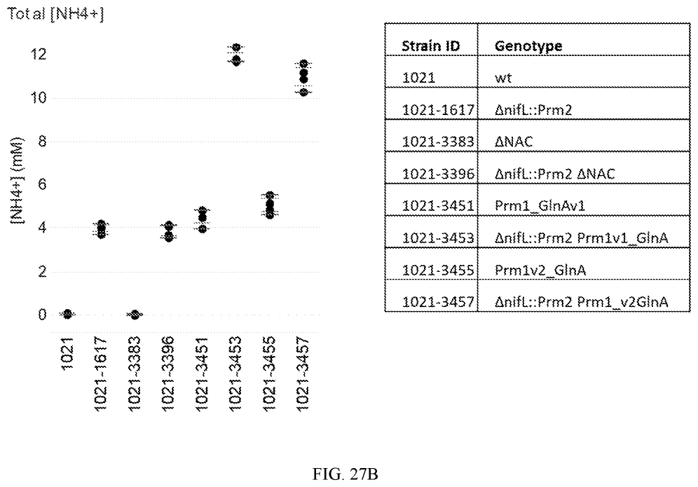

The mutant strains were subjected to AMM and ARA assays. The results are shown in FIGS. 24A-B, and 25A-B. Results for NAC mutants in the CI1021 background are provided in FIGS. 26A-B and 27A-B.

TABLE 3
Strains described herein

| Strain | | Mutagenic DNA | Chromosomal | Curing |
|---|---|---|---|---|
| ID | Lineage | Description | Genotype | Status |
| CI1021 | CI1021 | Wildtype parent | WT | N/A |
| | Kosakania | | | |
| | pseudosacchari | | | |
| 1021- | | Disruption of nifL gene with a | ΔnifL::nifA::Prm1, | |
| 1615 | | fragment of the region upstream | ΔglnE-AR_KO2 | |
| | | of the lpp gene (Prm1) inserted | | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (ΔglnE- | | |
| | | AR_KO2). | | |
| 1021- | 1021 | Disruption of nifL gene with a | ΔnifL::Prm2 | |
| 1617 | | 197 bp fragment of the region | | |
| | | upstream of a hypothetical gene | | |
| | | in CI1021 with strong | | |
| | | constitutive expression (Prm2) | | |
| | | inserted upstream of nifA. | | |
| 1021- | | A fragment of the region | fhuF::Prm1 | |
| 3545 | | upstream of the lpp gene (Prm1) | | |
| | | inserted upstream of fhuF. | | |
| 1021- | | Deletion of the entire iscR CDS | ΔiscR | |
| 3553 | | | | |
| 1021- | | A fragment of the region | sbnA::Prm1 | |
| 3555 | | upstream of the lpp gene (Prm1) | | |
| | | inserted upstream of sbnA. | | |
| 1021- | | A fragment of the region | yusV1::Prm1 | |
| 3559 | | upstream of the lpp gene (Prm1) | | |
| | | inserted upstream of yusV1. | | |
| 1021- | | A fragment of the region | yusV2::Prm1 | |
| 3563 | | upstream of the lpp gene (Prm1) | | |
| | | inserted upstream of yusV2. | | |
| 1021- | | Disruption of nifL gene with a | ΔnifL::nifA::Prm1, | |
| 3547 | | fragment of the region upstream | ΔglnE-AR_KO2, | |
| | | of the lpp gene (Prm1) inserted | fhuF::Prm1 | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (ΔglnE- | | |
| | | AR_KO2). Prm1 inserted | | |
| | | upstream of fhuF. | | |
| 1021- | | Disruption of nifL gene with a | ΔnifL::nifA::Prm1, | |
| 3591 | | fragment of the region upstream | ΔglnE-AR_KO2, | |
| | | of the lpp gene (Prm1) inserted | iscR | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (ΔglnE- | | |
| | | AR_KO2). Deletion of iscR | | |
| | | CDS. | | |
| 1021- | | Disruption of nifL gene with a | ΔnifL::nifA::Prm1, | |
| 3557 | | fragment of the region upstream | ΔglnE-AR_KO2, | |
| | | of the lpp gene (Prm1) inserted | sbnA::Prm1 | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (ΔglnE- | | |
| | | AR_KO2). Prm1 inserted | | |
| | | upstream of sbnA. | | |
| 1021- | | Disruption of nifL gene with a | ΔnifL::nifA::Prm1, | |
| 3561 | | fragment of the region upstream | ΔglnE-AR_KO2, | |
| | | of the lpp gene (Prm1) inserted | yusV1::Prm1 | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (ΔglnE- | | |
| | | AR_KO2). Prm1 inserted | | |
| | | upstream of yusV1. | | |
| 1021- | | Disruption of nifL gene with a | ΔnifL::nifA::Prm1, | |
| 3565 | | fragment of the region upstream | ΔglnE-AR_KO2, | |
| | | of the lpp gene (Prm1) inserted | yusV2: :Prm1 | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (ΔglnE- | | |
| | | AR_KO2). Prm1 inserted | | |
| | | upstream of yusV2. | | |
| 1021- | | A fragment of the region | sodA::Prm1 | |
| 3515 | | upstream of the lpp gene (Prm1) | | |
| | | inserted upstream of sodA. | | |
| 1021- | | Disruption of nifL gene with a | ΔnifL::nifA::Prm1, | |
| 3517 | | fragment of the region upstream | ΔglnE-AR_KO2, | |
| | | of the lpp gene (Prm1) inserted | sodA::Prm1 | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (ΔglnE- | | |
| | | AR_KO2). Prm1 inserted | | |
| | | upstream of sodA. | | |
| 1021- | | A fragment of the region | sodB::Prm1 | |
| 3519 | | upstream of the lpp gene (Prm1) | | |
| | | inserted upstream of sodB. | | |
| 1021- | | Disruption of nifL gene with a | ΔnifL::nifA::Prm1, | |
| 3521 | | fragment of the region upstream | ΔglnE-AR_KO2, | |
| | | of the lpp gene (Prm1) inserted | sodB::Prm1 | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (ΔglnE- | | |
| | | AR_KO2). Prm1 inserted | | |
| | | upstream of sodB. | | |
| 1021- | | A fragment of the region | sodC::Prm1 | |
| 3523 | | upstream of the lpp gene (Prm1) | | |
| | | inserted upstream of sodC. | | |
| 1021- | | Disruption of nifL gene with a | ΔnifL::nifA::Prm1, | |
| 3525 | | fragment of the region upstream | ΔglnE-AR_KO2, | |
| | | of the lpp gene (Prm1) inserted | sodC::Prm1 | |
| | | upstream of nifA. Deletion of the | | |
| | | 1647bp after the start codon of | | |
| | | the glnE gene containing the | | |
| | | adenylyl-removing domain of | | |
| | | glutamate-ammonia-ligase | | |
| | | adenylyltransferase (glnE- | | |
| | | AR_KO2). Prm1 inserted | | |
| | | upstream of sodC. | | |
| 1021- | 1021 | Deletion of the 918bp | ΔNAC | |
| 3383 | | NAC(cynR) gene from start to | | |
| | | stop codon | | |
| 1021- | | Deletion of the 918bp | ΔnifL::Prm2 | |
| 3396 | | NAC(oxyR) gene from start to | ΔNAC | |
| | | stop | | |
| CI137 | CI137 | Wildtype parent K. variicola | WT | N/A |
| 137- | | Disruption of nifL gene with a | ΔnifL::PrminfC | |
| 1036 | | fragment of the region upstream | | |
| | | of the infC gene inserted | | |
| | | (PrminfC) upstream of nifA. | | |
| 137- | Mutant of | Disruption of nifL gene with a | ΔnifL::Prm1.2 | cured |
| 2084 | CI137 | fragment of the region upstream | ΔglnE-AR_KO2 | |
| | | of the cspE gene inserted | | |
| | | (Prm1.2) upstream of nifA. | | |
| | | Deletion of the 1647bp after the | | |
| | | start codon of the glnE gene | | |
| | | containing the adenylyl-removing | | |
| | | domain of glutamate-ammonia- | | |
| | | ligase adenylyltransferase | | |
| | | (ΔglnE-AR_KO2). | | |
| 137- | | Disruption of nifL gene with a | ΔnifL::Prm1.2 | |
| 2448 | | fragment of the region upstream | | |
| | | of the cspE gene inserted | | |
| | | (Prm1.2) upstream of nifA. | | |
| 137- | | Disruption of nifL gene with a | ΔnifL::Prm1.2 | |
| 2512 | | fragment of the region upstream | ΔglnE-AR_KO2, | |
| | | of the cspE gene inserted | ΔgltA2 | |
| | | (Prm1.2) upstream of nifA. | | |
| | | Deletion of the 1647bp after the | | |
| | | start codon of the glnE gene | | |
| | | containing the adenylyl-removing | | |
| | | domain of glutamate-ammonia- | | |
| | | ligase adenylyltransferase | | |
| | | (ΔglnE-AR_KO2). Deletion of | | |
| | | gltA2 CDS | | |
| 137- | | Disruption of nifL gene with a | ΔnifL::Prm1.2 | |
| 2534 | | fragment of the region upstream | ΔglnE-AR_KO2, | |
| | | of the cspE gene inserted | Prm1.2::ptsH | |
| | | (Prm1.2) upstream of nifA. | | |
| | | Deletion of the 1647bp after the | | |
| | | start codon of the glnE gene | | |
| | | containing the adenylyl-removing | | |
| | | domain of glutamate-ammonia- | | |
| | | ligase adenylyltransferase | | |
| | | (ΔglnE-AR_KO2). Prm1.2 | | |
| | | inserted upstream of ptsH | | |
| 137- | | Deletion of all but 83bp of the 5′ | ΔnifL_with_RBS | |
| 2837 | | end of nifL | | |
| 137- | 137 | Fragment of the region upstream | Prm1.2_NAC | |
| 3004 | | of the cspE gene (Prm1.2) | | |
| | | inserted upstream of the start | | |
| | | codon of the NAC (oxyR) gene. | | |
| 137- | 137-1036 | Fragment of the region upstream | ΔnifL::PinfC | |
| 3006 | | of the cspE gene (Prm1.2) | Prm1.2_NAC | |
| | | inserted upstream of the start | | |
| | | codon of the NAC (oxyR) gene. | | |
| 137- | 137-2084 | Fragment of the region upstream | glnE_KO2 ΔnifL- | |
| 3008 | | of the cspE gene (Prm1.2) | Prm1.2 | |
| | | inserted upstream of the start | Prm1.2_NAC | |
| | | codon of the NAC (oxyR) gene. | | |
| 137- | 137-2448 | Fragment of the region upstream | nifL-Prm1.2 | |
| 3012 | | of the cspE gene (Prm1.2) | Prm1.2_NAC | |
| | | inserted upstream of the start | | |
| | | codon of the NAC (oxyR) gene. | | |
| 137- | 137-2837 | Fragment of the region upstream | ΔnifL_with-3′- | |
| 3014 | | of the cspE gene (Prm1.2) | RBS Prm1.2_NAC | |
| | | inserted upstream of the start | | |
| | | codon of the NAC (oxyR) gene. | | |
| 137- | | A fragment of the region | Prm1.2::fhuF | |
| 3161 | | upstream of the cspE gene | | |
| | | inserted (Prm1.2) upstream of | | |
| | | fhuF. | | |
| 137- | | Deletion of iscR CDS | ΔiscR | |
| 3214 | | | | |
| 137- | | Disruption of nifL gene with a | ΔnifL::Prm1.2 | |
| 3193 | | fragment of the region upstream | ΔglnE-AR_KO2, | |
| | | of the cspE gene inserted | Prm1.2::fhuF | |
| | | (Prm1.2) upstream of nifA. | | |
| | | Deletion of the 1647bp after the | | |
| | | start codon of the glnE gene | | |
| | | containing the adenylyl-removing | | |
| | | domain of glutamate-ammonia- | | |
| | | ligase adenylyltransferase | | |
| | | (ΔglnE-AR_KO2). Prm1.2 | | |
| | | inserted in a region upstream of | | |
| | | fhuF | | |
| 137- | | Disruption of nifL gene with a | ΔnifL::Prm1.2 | |
| 3195 | | fragment of the region upstream | ΔglnE-AR_KO2, | |
| | | of the cspE gene inserted | ΔiscR | |
| | | (Prm1.2) upstream of nifA. | | |
| | | Deletion of the 1647bp after the | | |
| | | start codon of the glnE gene | | |
| | | containing the adenylyl-removing | | |
| | | domain of glutamate-ammonia- | | |
| | | ligase adenylyltransferase | | |
| | | (ΔglnE-AR_KO2). Deletion of | | |
| | | iscR CDS | | |
| 137- | | A fragment of the region | Prm1.2::sodA | |
| 3120 | | upstream of the cspE gene | | |
| | | inserted (Prm1.2) upstream of | | |
| | | sodA. | | |
| 137- | | A fragment of the region | Prm1.2::sodB | |
| 3122 | | upstream of the cspE gene | | |
| | | inserted (Prm1.2) upstream of | | |
| | | sodB. | | |
| 137- | | A fragment of the region | Prm1.2::sodC | |
| 3124 | | upstream of the cspE gene | | |
| | | inserted (Prm1.2) upstream of | | |
| | | sodC. | | |
| 137- | | Disruption of nifL gene with a | ΔnifL::Prm1.2 | |
| 3183 | | fragment of the region upstream | ΔglnE-AR_KO2, | |
| | | of the cspE gene inserted | Prm1.2::sodA | |
| | | (Prm1.2) upstream of nifA. | | |
| | | Deletion of the 1647bp after the | | |
| | | start codon of the glnE gene | | |
| | | containing the adenylyl-removing | | |
| | | domain of glutamate-ammonia- | | |
| | | ligase adenylyltransferase | | |
| | | (ΔglnE-AR_KO2). Prm1.2 | | |
| | | inserted in a region upstream of | | |
| | | sodA | | |
| 137- | | Disruption of nifL gene with a | ΔnifL::Prm1.2 | |
| 3187 | | fragment of the region upstream | ΔglnE-AR_KO2, | |
| | | of the cspE gene inserted | Prm1.2::sodC | |
| | | (Prm1.2) upstream of nifA. | | |
| | | Deletion of the 1647bp after the | | |
| | | start codon of the glnE gene | | |
| | | containing the adenylyl-removing | | |
| | | domain of glutamate-ammonia- | | |
| | | ligase adenylyltransferase | | |
| | | (ΔglnE-AR_KO2). Prm1.2 | | |
| | | inserted in a region upstream of | | |
| | | sodC | | |
| 137- | | Deletion of the 918bp | ΔNAC | |
| 3322 | | NAC(oxyR) gene from start to | | |
| | | stop | | |
| 137- | | Deletion of the 918bp | ΔnifL::PinfC | |
| 3324 | | NAC(oxyR) gene from start to | ΔNAC | |
| | | stop | | |
| 137- | | Deletion of the 918bp | glnE_KO2 ΔnifL- | |
| 3326 | | NAC(oxyR) gene from start to | Prm1.2 ΔNAC | |
| | | stop | | |

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A genetically engineered bacterium comprising a modification in a gene selected from the group consisting of: NAC, gltA, pga, ptsH, fimA1, fimA2, fimA3, fimA4, iscR, tonB, yusV1, yusV2, yusV3, yusV4, sbnA, fhuF, sodA, sodB, sodC, iaaA, sdiA, wzxE, bolA, FNR, arcA, arcB, rpoS, treA, treB, phoP, phoQ, yjjPB, ychM, dauA, actP, yieL1, yieL2, yieL3, yieL4, pgab, rafA, melA, uidA, manA, abfA, abnA, and lacZ.

2.-4. (canceled)

5. The genetically engineered bacterium of claim 1, wherein said genetically engineered bacterium is a genetically engineered diazotrophic bacterium.

6. (canceled)

7. The genetically engineered bacterium of claim 1, wherein said genetically engineered bacterium is non-intergeneric.

8. The genetically engineered bacterium of claim 1, wherein said genetically engineered bacterium is able to fix atmospheric nitrogen in the presence of exogenous nitrogen.

9. The genetically engineered bacterium of claim 1, wherein said genetically engineered bacterium further comprises a modification in a nitrogen fixation genetic network, a modification in a nitrogen fixation assimilation genetic network, or a modification in the nitrogen fixation network and the nitrogen fixation assimilation genetic network.

10. The genetically engineered bacterium of claim 9, wherein said modification in a nitrogen fixation genetic network comprises a modification in NifA, NifL, NifH, or any combination thereof, or a modification that results in increased expression of Nif cluster genes.

11. The genetically engineered bacterium of claim 10, wherein said modification in NifA results in increased expression of NifA, said modification in NifL results in decreased expression of NifL, or said modification in NifH results in increased expression of NifH.

12.-15. (canceled)

16. The genetically engineered bacterium of claim 9, wherein said modification in a nitrogen assimilation genetic network comprises a modification that results in decreased activity of GlnE or results in a decreased activity of amtB.

17.-18. (canceled)

19. A method of increasing the amount of atmospheric derived nitrogen in a plant, the method comprising contacting said plant with a plurality of said genetically engineered bacteria of claim 1.

20. The method of claim 19, wherein contacting said plant with a plurality of said genetically engineered bacteria comprises applying said plurality of genetically engineered bacteria to a seed or seedling of said plant.

21.-24. (canceled)

25. The method of claim 19, wherein contacting said plant with a plurality of said genetically engineered bacteria comprises applying said plurality of genetically engineered bacteria to said plant between about one month and about eight months after germination, between two months and eight months after germination, between about one month to about three month after germination, or between about three months to about six months after germination.

26.-28. (canceled)

29. The method of claim 19, wherein said plant is a cereal plant.

30. The method of claim 19, wherein said plant is a corn plant.

31. The method of claim 19, wherein said plant is a rice plant.

32. The method of claim 19, wherein said plant is a wheat plant.

33. The method of claim 19, wherein said plant is a soy plant.

34. A composition comprising a seed and a seed coating, wherein the seed coating comprises a plurality of said genetically engineered bacteria of claim 1.

35. The composition of claim 34, wherein said seed is a cereal seed.

36. (canceled)

37. A composition comprising a plant and a plurality of said genetically engineered bacteria of claim 1, wherein said plant optionally is a seedling.

38. (canceled)

39. The composition of claim 37, wherein said plant is a cereal plant.

40. (canceled)
